SEQUENCE LISTING (16 sequences)

SEQ ID NO: 1 - DNA, 26 bp, synthetic construct
  source 1..26: mol_type = other DNA
tctattactaccacttcctaaatcaa

SEQ ID NO: 2 - DNA, 28 bp, synthetic construct
  source 1..28: mol_type = other DNA
tgagatgtttgttagtttattattttgg

SEQ ID NO: 3 - DNA, 22 bp, synthetic construct
  source 1..22: mol_type = other DNA
tcatcttctcccaccaaatttc

SEQ ID NO: 4 - DNA, 22 bp, synthetic construct
  source 1..22: mol_type = other DNA
tagagggtagtttttaagaggg

SEQ ID NO: 5 - DNA, 25 bp, synthetic construct
  source 1..25: mol_type = other DNA
attaaccaacctcttccatattctt

SEQ ID NO: 6 - DNA, 24 bp, synthetic construct
  source 1..24: mol_type = other DNA
ttttagttttgggaggggttgttt

SEQ ID NO: 7 - DNA, 25 bp, synthetic construct
  source 1..25: mol_type = other DNA
ctaccaaaattaaaaaccaacccac

SEQ ID NO: 8 - DNA, 28 bp, synthetic construct
  source 1..28: mol_type = other DNA
gggttatatgatagtttatagggttatt

SEQ ID NO: 9 - DNA, 79 bp, synthetic construct
  source 1..79: mol_type = other DNA
acgcactctgatatgtgatctctctcttttcctcctcctccgttgttgttgttgagagagatcacatatcagagtgcgt

SEQ ID NO: 10 - DNA, 16 bp, synthetic construct
  source 1..16: mol_type = other DNA
cacatatcagagtgcg

SEQ ID NO: 11 - DNA, 79 bp, synthetic construct
  source 1..79: mol_type = other DNA
actcacagtctgtgtgtatctctctcttttcctcctcctccgttgttgttgttgagagagatacacacagactgtgagt

SEQ ID NO: 12 - DNA, 16 bp, synthetic construct
  source 1..16: mol_type = other DNA
acacacagactgtgag

SEQ ID NO: 13 - DNA, 79 bp, synthetic construct
  source 1..79: mol_type = other DNA
actctcacgagatgtgtatctctctcttttcctcctcctccgttgttgttgttgagagagatacacatctcgtgagagt

SEQ ID NO: 14 - DNA, 16 bp, synthetic construct
  source 1..16: mol_type = other DNA
acacatctcgtgagag

SEQ ID NO: 15 - DNA, 79 bp, synthetic construct
  source 1..79: mol_type = other DNA
acgcagcgctcgactgtatctctctcttttcctcctcctccgttgttgttgttgagagagatacagtcgagcgctgcgt

SEQ ID NO: 16 - DNA, 16 bp, synthetic construct
  source 1..16: mol_type = other DNA
acagtcgagcgctgcg

(19) United States
(12) Patent Application Publication
(10) Pub. No.: US 2024/0271202 A1
(43) Pub. Date: Aug. 15, 2024

(54) ULTRA HIGH-FIDELITY SINGLE MOLECULE SEQUENCING

(71) Applicant: New York University, New York, NY (US)

(72) Inventor: Gilad Evrony, New York, NY (US)

(21) Appl. No.: 18/428,519

(22) Filed: Jan. 31, 2024

Related U.S. Application Data
(60) Provisional application No. 63/442,370, filed on Jan. 31, 2023.

(51) Int. Cl. C12Q 1/6869; C12Q 1/25; C12Q 1/6806

(52) U.S. Cl. CPC C12Q 1/6869; C12Q 1/25; C12Q 1/6806; G01N 2333/9015

(57) ABSTRACT

Provided are compositions and methods for improved accuracy during DNA sequencing. The method can be performed without amplification prior to sequencing. The compositions and methods are used for determining single and double stranded sequences, and for accurately determining mosaic double strand mutations and single strand nucleotide changes.

### CROSS REFERENCE TO RELATED APPLICATION

This application claims the benefit of U.S. provisional application No. 63/442,370, filed Jan. 31, 2023, the entire disclosure of which is incorporated herein by reference.

### STATEMENT REGARDING FEDERALLY SPONSORED RESEARCH OR DEVELOPMENT

This invention was made with government support under R21 HD105910, OD028158, and NS132024 awarded by the National Institutes of Health. The government has certain rights in the invention.

### FIELD

The disclosure relates to compositions and methods that improve polynucleotide sequencing.

### SEQUENCE LISTING STATEMENT

The instant application contains a Sequence Listing which is submitted in .xml format and is hereby incorporated by reference in its entirety. Said .xml file is named “058636_00665.xml”, was created on Jan. 31, 2024, and is 15,390 bytes in size.

### RELATED INFORMATION

Mosaic mutations are ubiquitous in the body and accumulate throughout life in every cell^{1,2}. Most mosaic mutations begin as nucleotide mismatches or damage in only one of the two strands of the DNA double helix³⁻⁵. When these single-strand DNA (ssDNA) events are mis-repaired, or when they are replicated during the cell cycle prior to repair, they then become permanent double-strand DNA (dsDNA) mosaic mutations⁴. However, these ssDNA events, which are the origin of most mutations in the body, have remained invisible to current mutation profiling methods, which only reliably detect dsDNA mutations. This is because all current methods for profiling mosaic mutations-single-cell genome sequencing⁶⁻⁸, in vitro cloning of single cells^{9,10}, microdissection or biopsy of clonal populations^{11,12}, and duplex sequencing¹³⁻¹⁶ amplify the original DNA molecules prior to sequencing, either prior to or on the sequencer itself. Amplification of DNA prior to sequencing masks true ssDNA events by either transforming existing ssDNA mismatches and damage to dsDNA mutations, or by introducing artifactual ssDNA mismatches and damage¹⁷.

Mosaic dsDNA mutations are the result of the interaction between ssDNA mismatch and damage events, DNA repair, and DNA replication^{4,18}. For example, dsDNA mutational signatures (i.e., the sequence contexts of mutations) may not reflect the patterns of the originating ssDNA events, but rather only that of the ssDNA events that are mis-repaired or unrepaired prior to replication⁵. dsDNA mutation profiling also does not resolve on which strands the initiating mutational processes are occurring. Therefore, an improved understanding of the process of mutation requires profiling of ssDNA mismatches and damage^{4,19}. The present disclosure relates to an ongoing need for improved approaches to DNA sequencing.

### BRIEF SUMMARY

The present disclosure provides, among other aspects, compositions and methods that provide for improved polynucleotide sequencing. The disclosure includes but is not limited to determination of mosaic mutations and single-strand DNA mismatches and damage which are ubiquitous in the body and occur in every cell^{1,2}. Most mosaic mutations begin as nucleotide mismatches or damage in only one of the two strands of the DNA double helix³⁻⁵. When these single-strand DNA (ssDNA) events are mis-repaired, or when they are replicated during the cell cycle prior to repair, they then become permanent double-strand DNA (dsDNA) mosaic mutations⁴. However, these ssDNA events, which are the origin of most mutations in the body, have remained invisible to previously available mutation profiling methods, which only reliably detect dsDNA mutations. Without intending to be bound by any particular theory it is considered that this is because all previous methods for profiling mosaic mutations-single-cell genome sequencing⁶⁻⁸, in vitro cloning of single cells^{9,10}, microdissection or biopsy of clonal populations^{11,12}, and duplex sequencing¹³⁻¹⁶—amplify the original DNA molecules prior to sequencing, either prior to or on the sequencer itself. Amplification of DNA prior to sequencing masks true ssDNA events by either transforming existing ssDNA mismatches and damage to dsDNA mutations, or by introducing artifactual ssDNA mismatches and damage¹⁷.

Mosaic dsDNA mutations are the result of the interaction between ssDNA mismatch and damage events, DNA repair, and DNA replication^{4,18}. For example, dsDNA mutational signatures (i.e., the sequence contexts of mutations) may not reflect the patterns of the originating ssDNA events, but rather only that of the ssDNA events that are mis-repaired or unrepaired prior to replication⁵. dsDNA mutation profiling also does not resolve on which strands the initiating mutational processes are occurring. Therefore, a complete understanding of the process of mutation requires profiling of ssDNA mismatches and damage^{4,19}. This disclosure relates in part to the ssDNA origins of mosaic mutations, and in certain aspects provides compositions and methods for direct sequencing of single DNA molecules without any prior amplification that achieves, for substitutions, single-molecule fidelity detection of dsDNA mutations simultaneously with ssDNA mismatches and damage, and for the first time, ultra-high fidelity long-read sequencing. In non-limiting examples, the disclosure provides single-molecule sequencing methods that achieve single-molecule fidelity for single-base substitutions when present in either one or both strands of the DNA.

The method also detects single-strand cytosine deamination events, one of the most prevalent types of DNA damage. The described methods facilitate detection of initiating single-strand DNA mismatches and damage and provide a basis for previously unavailable studies of mutagenic processes in cultured cells and primary tissues in a variety of contexts, with particular applicability in cancer and aging. A described method may be referred to herein from time to time as Hairpin Duplex Enhanced Fidelity Sequencing (HiDEF-seq).

### BRIEF DESCRIPTION OF THE FIGURES

The patent or application file contains at least one drawing executed in color. Copies of this patent or patent application publication with color drawing(s) will be provided by the Office upon request and payment of the necessary fee.

Certain figures show consecutive nucleotide triplets. These are not to be interpreted to be consecutive sequences that are more than three nucleotides long.

FIGS. 1a-1g. Overview of method. a, Schematic of library preparation and sequencing. A-tailing is performed with a polymerase, dATP and non-A dideoxynucleotides to block residual nicks¹⁷ (not illustrated), except for fragmented DNA samples that utilize only dideoxynucleotides (without dATP) in this step to avoid misincorporation of dATP at these samples' more numerous residual nick sites (FIGS. 9f and 10d, and Methods). The latter samples are ligated to blunt adapters. Sequencing reads are reverse complements of the template molecule. b, Histogram of the average number of passes per strand (Methods) for representative HiDEF-seq samples (n=51) and standard PacBio HiFi samples (n=10). The average percentage of molecules with ≥5 and >20 passes per strand is: 99.8% and 70% for HiDEF-seq, respectively, and 78.7% and 0.1% for HiFi, respectively. b, Plot shows HiDEF-seq molecules output by the primary data processing step of the analysis pipeline. X-axis square brackets and parentheses signify inclusion and exclusion of interval endpoints, respectively. c, dsDNA mutation burdens in sperm samples (left to right: SPM-1013, SPM-1002, SPM-1004, SPM-1020, SPM-1060) profiled by both HiDEF-seq v2 and NanoSeq, compared for each age (yo, years old) to paternally-phased de novo mutations in children from a prior study of 2,976 trios²⁰. d, dsDNA mutation burdens versus age measured by HiDEF-seq v2 in samples from individuals without cancer predisposition. Dashed lines (liver, kidney, blood): weighted least-squares linear regression. Dotted line (neurons): these only connect two data points to aid visualization of burden difference, since regression cannot be performed with two samples. e, Comparison of HiDEF-seq versus NanoSeq dsDNA mutations per base pair for samples profiled by both methods. Samples (top to bottom in legend) are: SPM-1013, SPM-1002, SPM-1004, SPM-1020, SPM-1060, 1443, 1105, 6501, 63143. Note, all samples except for 63143 (POLE p.M444K) are from individuals without a cancer predisposition syndrome. Dashed line diagonal, y=x, is the expectation for concordance. f, Comparison of HiDEF-seq versus NanoSeq ssDNA calls per base for samples profiled by both methods. These are the same samples as in (e). g, Comparison of HiDEF-seq versus NanoSeq ssDNA calls per base, separated by call type. For each call type (i.e., C>A, C>G, etc.), each bar represents a different sperm sample. Samples for each call type, from left to right, are SPM-1013, SPM-1002, SPM-1004, SPM-1020, SPM-1060. b, Error bars: standard deviation. c-f, Error bars: Poisson 95% confidence intervals. c, Box plots: middle line-median, boxes—1st and 3rd quartiles, whiskers—5% and 95% quantiles. e-g, yo, years old; mo, months old.

FIGS. 2a-2g. ssDNA call burdens and patterns in cancer-predisposition syndromes. a, Burdens of ssDNA calls in blood (B), fibroblasts (F), and lymphoblastoid cell lines (L) from individuals without and with cancer predisposition syndromes. Call burdens are corrected for trinucleotide context opportunities and detection sensitivity (Methods). ***, p=8·10⁻¹¹ for mismatch repair versus non-cancer predisposition samples and p<10⁻¹⁵ for polymerase proofreading versus non-cancer predisposition samples (Poisson rates ratio test, using combined counts of calls and interrogated bases from each group). Results were still significant when including only blood samples. From left to right, non-cancer predisposition samples are: 5203, 1105, 1301, 6501, 1901, GM12812, GM02036, GM03348; cancer predisposition samples are: GM16381, GM01629, GM28257, 55838, 58801, 57627, 1400, 1324, 1325, 60603, 59637, 57615, 63143 (L), 63143 (B), CC-346-253, CC-388-290, CC-713-555. For cancer predisposition samples, the affected genes are in the same left-right order as for cancer predisposition samples in (b). b, Fraction of ssDNA call burdens by context. Call burdens are corrected for trinucleotide context opportunities. We include only non-cancer predisposition samples with >30 ssDNA calls (1105, 1301, 1901, GM12812, GM03348) for reliable fraction estimates. The cancer predisposition syndrome samples are in the same order as in (a). However, the cancer predisposition sample GM16381 (XPC) with <30 ssDNA calls is included for completeness in showing all cancer predisposition samples. c,d, Representative ssDNA (c) and dsDNA (d) call spectra for POLE sample 57615, corrected for trinucleotide context opportunities. Parentheses show total number of calls. e, Top, ssDNA mismatch signature SBS10ss extracted from all POLE samples. The signature was extracted de novo while simultaneously fitting SBS30ss* (see FIG. 3e). Middle, SBS10ss projected to central pyrimidine context by summing central pyrimidine and central purine values to allow comparison to dsDNA signatures. Bottom, dsDNA mutational signature (sum of SBSD and SBSE) extracted de novo from all POLE samples, while simultaneously fitting SBS1 and SBS5. f, Fraction of ssDNA calls attributed to each ssDNA signature in POLE samples (left to right): 59637, 57615, and 63143 lymphoblastoid cell lines, and 63143 blood. Protein-level POLE mutation is annotated below. See FIG. 3e for details of SBS30ss*. g, In POLE samples, AGA>ATA ssDNA mismatches and AGA>ATA dsDNA mutations occur more often on the non-reference (−) than on the reference (+) strand in regions where the non-reference strand is synthesized more frequently in the leading direction (i.e., positive fork polarity), based on replication timing data (Methods). Reference (+) strand refers to the plus strand of the human reference genome. See FIG. 12e for plots of dsDNA mutations separated by fork polarity quantiles (rather than positive versus negative polarity), which cannot be plotted for ssDNA mismatches due to the low number of ssDNA mismatches per quantile. Y-axis is the ‘strand ratio’, calculated as the fraction of all AGA>ATA non-reference strand events that have the specified fork polarity divided by the fraction of all AGA>ATA reference strand events that have the specified fork polarity. For ssDNA analysis, the strand ratio is calculated using the ssDNA mismatches of all POLE samples, since there are not enough ssDNA mismatches to quantify this reliably for each sample separately. For dsDNA analysis, strand ratios were calculated for each sample separately, and the plot shows average and standard deviation (error bars) across these samples. Dashed line at 1.0 is the expected ratio in the absence of strand asymmetry. *, p=0.015 (chi-squared test, n=73 ssDNA AGA>ATA mismatches); ***, p<10⁻¹⁵ (chi-squared test of all 3,871 dsDNA AGA>ATA mutations across all POLE samples). An analysis excluding mismatches and mutations overlapping genes, to exclude biases due to transcription strand, was still significant for dsDNA mutations (p<10⁻¹⁵) but not for ssDNA mismatches, but this analysis has significantly reduced power due to the 55% reduction in the number of ssDNA mismatches remaining for analysis. a,b, See further disease and sample details, including genotypes, in Supplementary Tables 1-2. a, Error bars, Poisson 95% confidence intervals.

FIGS. 3a-3h. ssDNA damage signatures in sperm and heat treatment. a, Spectrum of all ssDNA calls of non-cancer predisposition (healthy) blood samples (1 sample each from individuals 1105, 1301, 5203, 6501, and 5 samples from individual 1901). Cosine similarity to the dsDNA COSMIC signature SBS30 is calculated after projecting the ssDNA 192-context spectrum to 96-context central pyrimidine spectrum (by summing values of central pyrimidine and their reverse complement central purine contexts). b, dsDNA mutation and ssDNA call burdens of heat-treated DNA. The percentage of ssDNA sequencing calls that are C>T are annotated above each sample. c, Spectra of ssDNA calls for representative sperm and heat-treated blood DNA samples, and COSMIC SBS30 for comparison. d, Cosine similarity of all ssDNA calls of each individual sample to COSMIC SBS30, after projecting ssDNA calls to 96 central pyrimidine trinucleotide contexts. e, SBS30ss* obtained by de novo signature extraction from central pyrimidine ssDNA calls of sperm and heat-treated samples. Cosine similarity to SBS30 is calculated after projecting to 96-context central pyrimidine spectrum. f, Schematic of pulse width (PW) and interpulse duration (IPD) measured by the sequencer for each incorporated base. g, Average ratio of pulse widths of C>T calls, and 30 base pairs flanking the call, of each molecule with the call relative to molecules aligning to the same locus without the call. Data shows the average of the ratios for all ssDNA C>T calls in sperm samples (n=1799 calls), blood DNA samples that were heat-treated at 72 C for 3 or 6 hours (n=626 calls), and dsDNA C>T mutations in a larger set of samples (non-heat treated blood DNA, 56 C and 72 C heat treated blood DNA, sperm, kidney, and liver; n=1217 mutations). The distinct profile of ssDNA C>T calls versus dsDNA C>T mutations, most notably at positions +1 and +2 (star), indicates the ssDNA calls are damaged cytosines rather than cytosine to thymine mutations. h, Heat map of average pulse width ratios for C>T ssDNA calls and C>T dsDNA mutations for positions −1 to +6. Unbiased clustering of kinetic profiles (dendrogram) separates ssDNA from dsDNA calls and from kinetic profiles after randomizing labels of molecules with and without the calls. ssDNA ‘Blood, 72 C heat (3 h and 6 h)’ (h, hours): heat-treated blood DNA. dsDNA ‘Blood, heat’: blood DNA heat-treated at 56 C and 72 C (both 3 h and 6 h for each); dsDNA ‘Blood’: 4 samples, not heat treated. dsDNA ‘Kidney and liver’: 10 samples, not heat treated. Star indicates positions +1 and +2 that best discriminate ssDNA C>T damage from dsDNA C>T mutations. b, Error bars, Poisson 95% confidence intervals. *, p<0.005; ns, p>0.05; Poisson rates ratio test. a,c-e, Prior to plotting and analysis, HiDEF-seq spectra are corrected for trinucleotide context opportunities (Methods). g, Error bars, standard error of the mean.

FIGS. 4a-4c. ssDNA call burdens and patterns in healthy tissues. a, Fraction of ssDNA calls that are C>T (corrected for trinucleotide context opportunities) across all HiDEF-seq samples from healthy individuals and cell lines (i.e., excluding cancer-predisposition syndromes), versus the total ssDNA call burden. LCL, lymphoblastoid cell line. b, ssDNA call burden versus age across all HiDEF-seq v2 samples from healthy individuals (primary tissues). Dashed lines: weighted least-squares linear regression, with a 95% confidence interval (shaded ribbon) shown for the statistically significant association for liver. c, Fraction of ssDNA call burdens by context, after pooling calls of samples from healthy individuals and cell lines, separately for each tissue. Call burdens are corrected for trinucleotide context opportunities. The number of samples and calls for each tissue are listed above. See FIGS. 14d,e for ssDNA and dsDNA call burdens by context for individual samples, and FIG. 14f for ssDNA spectra for each tissue. b, Error bars, Poisson 95% confidence intervals.

FIGS. 5a-5e. Mitochondrial genome dsDNA mutation and ssDNA call burdens and patterns. a, dsDNA mutation burdens versus age in the mitochondrial genome of liver and kidney samples, including liver samples from which mitochondria were enriched. Dashed lines: weighted least-squares linear regression (p<0.0005 and p=0.003 for regression slope for liver and kidney, respectively), with a 95% confidence interval (shaded ribbon). b, dsDNA mutation burdens per year in the nuclear versus mitochondrial genome. Liver and kidney mitochondrial genome data is from the regressions in panel (a), which were similarly performed for the nuclear genome as well as for liver and kidney samples combined. P-value, comparing the nuclear versus mitochondrial genome, within each tissue type: ANOVA comparing two weighted least-squares linear regression models, one with and one without an ‘age×genome type’ interaction term (an estimate of the difference of the dsDNA mutation burden slope versus age depending on whether it is the nuclear or mitochondrial genome). c, dsDNA mutation spectra in liver and kidney samples for the mitochondrial genome heavy strand, separated by pyrimidine (top) and purine (bottom) contexts. d, ssDNA call burdens in the nuclear and mitochondrial genomes, combining calls for liver and kidney samples, including liver samples from which mitochondria were enriched (n=27 calls). P-value, ANOVA. e, Spectrum of ssDNA calls combined from liver and kidney samples, including samples profiled by HiDEF-seq v2 with A-tailing, as well as liver samples from which mitochondria were enriched.

FIGS. 6a-6f. HiDEF-seq library preparation and sequencing metrics. a, Representative DNA sizing electropherogram after Hpy166II restriction enzyme digestion (top) and after completion of the HiDEF-seq library preparation that includes steps to remove fragments <1 kb (bottom). b, Two-dimensional histogram heatmap for all molecules from a representative HiDEF-seq sequencing run of each molecule's longest strand read length (bp, base pairs) versus its total polymerase read length (PRL). Dashed line signifies the expected strand length distribution, and note peak PRL >100 kilobases. The red diagonal line reflects 18% of molecules with <1 strand pass, which is typical in PacBio sequencing. c, Histogram (200 bp bins) for representative HiDEF-seq samples (n=51) of molecule consensus sequence lengths (i.e., molecule sizes). Line and shaded region show average and standard deviation, respectively, across samples for each bin. The average of these samples' median lengths is 1.7 kb. d, Histogram as in panel (c), showing HiDEF-seq (n=51 samples) produces (by design) smaller molecule lengths than standard PacBio (HiFi) samples (n=10 samples). The average of samples' median lengths are 1.7 kb and 18.3 kb for HiDEF-seq and HiFi, respectively. e, Two-dimensional histogram of the number of passes (bin width of 5 passes) vs. consensus sequence lengths (200 bp bins) for molecules from the 51 representative HiDEF-seq samples plotted in panels (c,d). Bins are colored if there is at least one molecule in the bin. f, Box plots of the fraction of a molecule's consensus sequence bases (average of forward and reverse strands) that have the maximum predicted quality (quality=93, as predicted by ccs, Methods) versus the number of passes per strand, across all molecules. Samples are the same as in panel (d). Note: 93 is the quality required for HiDEF-seq analysis. This plot illustrates that the number of passes is a key determinant of consensus quality in both HiDEF-seq and HiFi. b, Plot generated by SMRT Link (Pacific Biosciences) software. c-e, The single molecule consensus sequence length is the average of the forward and reverse strand lengths. Bin values are normalized to the bin with the peak molecule count. e,f, The number of passes per strand is the average of the forward and reverse strand ‘ec’ tags (Methods). c-f, Plots show data of HiDEF-seq molecules that are output by the primary data processing step of the HiDEF-seq analysis pipeline and standard PacBio HiFi molecules that are output by the ccs HiFi pipeline (Methods). f, Box plot: middle line: median, boxes: 1st and 3rd quartiles, whiskers: 1.5× interquartile range or min/max value. X-axis: square brackets and parentheses signify inclusion and exclusion of interval endpoints, respectively.

FIG. 7. Schematic of analysis pipeline. Primary data processing steps (blue) are followed by call filtering (green) along with germline sequencing (orange), which is then followed by call burden and signature analysis (purple). See Methods for full details. On the left of primary data processing steps are the average percentage of molecules filtered by each step across 17 representative HiDEF-seq sequencing runs. Approximately half of molecules filtered by the ‘Generate consensus sequence’ step are molecules with less than 3 full length passes (default setting of the ccs tool that creates consensus sequences), and the other half are due to molecules with read quality (‘rq’ tag) <0.99. At the end of the call filtering steps are listed the percentage of bases filtered by the call filtering steps, calculated out of the total bases of molecules that pass primary data processing, for the same 17 representative HiDEF-seq sequencing runs. The filter for ‘low-quality genomic regions and gnomAD variants with allele frequency (AF) >0.1% in the population’ covers approximately 15% and 7% of the genome when using Illumina and PacBio germline sequencing data, respectively (i.e., when PacBio germline sequencing data is used, the pipeline uses less restrictive filters due to fewer genome alignment errors and artifacts). WGS, whole-genome sequencing.

FIGS. 8a-8l. Analysis thresholds and comparison of analyses using short-versus long-read germline sequencing. a, Histogram of predicted consensus sequence accuracy (‘rq’ tag, bin width=0.0001) for DNA molecules that pass primary data processing steps from 3 representative HiDEF-seq (v2) samples (sperm, 21 yo: SPM-1002; 39 yo: SPM-1004; 44 yo: SPM-1020; yo, years old). Note these are consensus sequence accuracies predicted by the ccs consensus calling software (Methods), which are used to filter low-quality molecules, but this does not reflect true accuracy which is significantly higher. b, Box plot of passes per strand for different consensus sequence accuracy bins, for molecules from the 3 samples included in panel (a), showing that higher minimum accuracies select for molecules with higher numbers of passes. c, Fraction of post-primary data processing molecules that are filtered (left plot) and fraction of post-primary data processing base pairs that remain for interrogation (right plot) using different minimum passes per strand and consensus sequence accuracy thresholds. Values show average of the 3 samples included in panel (a), after completing all steps of the mutation filtering pipeline. d,e, dsDNA mutation burdens for the 3 representative sperm samples included in the above panels using different minimum passes per strand and consensus sequence accuracy thresholds. Panel (e) shows data from (d) at consensus accuracy of 0.99 with Poisson 95% confidence intervals. These data illustrate stability of dsDNA mutation burden estimates at broad thresholds using sperm as the most stringent test of fidelity. f, Fraction of high-quality known germline variants detected using different minimum required fraction of molecule passes that detect the variant (filter applied separately to each strand). This value is used for sensitivity correction (Methods). Values show average of the 3 samples included in panel (a). g,h, dsDNA mutation burdens for the 3 representative sperm samples included in the above panels using different minimum required fraction of molecule passes that detect the variant (filter applied separately to each strand), after correcting for sensitivity (g), and using different minimum required distances from the end of the read (h). Panel (g) illustrates that correcting for sensitivity maintains stable burden estimates. The analysis pipeline requires a minimum of 10 bp from the ends of reads to remove rare alignment artifacts, even though this does not significantly alter burden estimates. i, ssDNA call burdens for the 3 representative sperm samples included in the above panels using different minimum passes per strand and consensus sequence accuracy thresholds. Plot shows a small decrease in ssDNA call burdens with a higher minimum required passes per strand at low consensus sequence accuracy thresholds, and convergence to similar burdens at high consensus sequence accuracy thresholds. Data shown with minimum fraction of 0.5 molecule passes that detect the variant. j, ssDNA call burdens for the 3 representative sperm samples included in the above panels using different minimum required fraction of molecule passes that detect the variant, after correcting for sensitivity. Data shown with minimum consensus sequence accuracy of 0.999 and minimum 20 passes per strand. k,l, Concordant dsDNA mutation and ssDNA call burdens obtained by HiDEF-seq v2 using short-read (Illumina) vs. long-read (PacBio, Pacific Biosciences) germline sequencing during analysis, for two samples (1301 and 1901 blood) for which both germline sequencing types are available. a-d,i, Consensus sequence accuracies are the average of forward and reverse strand accuracies. b, Box plot: middle line-median, boxes—1st and 3rd quartiles, whiskers—1.5× interquartile range or min/max value. X-axis: square brackets and parentheses signify inclusion and exclusion of interval endpoints, respectively. c-e,i, Threshold for minimum required passes per strand is applied to both strands. c-j, The symbols ‡ and § mark the final thresholds chosen for dsDNA and ssDNA analyses, respectively. c,f, Error bars: standard deviation; note, panel (f) error bars are small and therefore not well visualized. d,e,g-l, mutation and call burdens are corrected for sensitivity and trinucleotide context opportunities of the full genome relative to interrogated bases (Methods). e,g-h,j-l, Error bars: Poisson 95% confidence intervals.

FIG. 9a-9g. HiDEF-seq v1 dsDNA mutation burdens and removal of single-strand DNA artifacts by HiDEF-seq v2. a, dsDNA mutation burdens in two sperm samples (left to right: SPM-1004, SPM-1020) profiled by both HiDEF-seq v1 and NanoSeq, compared for each age (yo, years old) to paternally-phased de novo mutations in children from a prior study of 2,976 trios²⁰. b, dsDNA mutation burdens versus age, measured by HiDEF-seq v1. Dashed lines (liver, kidney): weighted least-squares linear regression. Dotted lines (blood, neurons): these only connect two data points to aid visualization of burden difference, since regression cannot be performed with two samples. c, Mutational signature contribution to dsDNA mutations detected in HiDEF-seq v1 samples. All samples, except blood from a 62 year-old individual (1901), were jointly analyzed with fitting of SBS1 and de novo extraction of one additional signature SBSB. The blood sample of the 62 year-old was analyzed separately together with 5 other HiDEF-seq v2 blood samples from this individual, due to identification of an additional signature SBSC, which matches SBS19 and SBS23. SBS19 has been associated with trichloropropane exposure, a pollutant found in drinking water²¹, and SBS23 has been associated with end-stage renal disease and dialysis. The latter is consistent with this individual's history of end-stage renal disease and a kidney transplant due to focal segmental glomerulosclerosis (Supplementary Table 1). Analysis of samples grouped by tissue type, excluding the 62 year-old blood sample, produced similar results. For de novo extracted signatures (SBSB and SBSC), the cosine similarities to the closest matching COSMIC signatures are shown in parentheses. Sperm samples and kidney and liver samples from an infant (1443) were not included here since the number of mutations is too low for reliable signature extraction. d, Burdens of dsDNA mutations (left) and ssDNA calls (right) of a blood sample (individual 1301) measured by HiDEF-seq v1 (without nick ligation), and HiDEF-seq v2 (with nick ligation). Nick ligation eliminates T>A ssDNA artifacts that match the illustrated GTTBVH motif. The motif was derived using the ggseqlogo R package using all T>A ssDNA calls (sequence relative to the template strand) from the HiDEF-seq v1 sample. Gray bar is calls matching the motif with log-odds score >2 calculated with the score_match function of the universalmotif R package. e,f, Proposed mechanism for the GTTBVH motif of ssDNA artifactual calls. The known GTNNAC motif of the Hpy166II restriction enzyme used in HiDEF-seq may arise if Hpy166II operates as a dimer (cut sites signified by triangles), with two monomers binding opposite strands with the GTTBVH motif with intersection (n) and union (U) combinatorial logic for the outer and inner 2 bases, respectively (e). ssDNA GT[T>A]BVH artifactual calls may arise from rare Hpy166II monomer nicking events followed by pyrophosphorolysis of the ‘T’ and addition of a mismatched ‘A’ during the Klenow dATP/ddBTP A-tailing reaction. Further extension with ddBTP does not occur due to the mismatch²² (f). g, HiDEF-seq v2's nick ligation increases library yield by 66% for post-mortem tissues, likely by repairing nicks in the original input DNA so that the molecules are not eliminated in the final nuclease treatment step. Number of samples per group (left to right): 8, 8, 5, 9 (**, p=0.002; ns, not significant; unpaired t-test). a, Box plots: middle line-median, boxes—1st and 3rd quartiles, whiskers—5% and 95% quantiles. a,b,d Error bars: Poisson 95% confidence intervals. g, Error bars: standard deviation.

FIGS. 10a-10i. HiDEF-seq v2 without A-tailing removes ssDNA artifacts of post-mortem tissues with fragmented DNA. a, Fraction of ssDNA calls that are T>A (corrected for trinucleotide context opportunities) versus the ssDNA T>A burden in all samples profiled with standard HiDEF-seq v2 (with A-tailing; i.e., Klenow reaction +dATP/+ddBTP) from healthy individuals and cell lines (i.e., excluding cancer-predisposition syndromes). Post-mortem kidney and liver consistently have the highest fraction of ssDNA calls that are T>A. b, Standard HiDEF-seq v2 (with A-tailing) ssDNA call spectrum for a liver sample with a high ssDNA T>A burden (6.8·10⁻⁷), corrected for trinucleotide context opportunities. Parentheses show total number of calls. c, Correlation between ssDNA T>A artifact burden and the input DNA's DNA Integrity Number measured by TapeStation electrophoresis²³ across all samples profiled with standard HiDEF-seq v2 (with A-tailing) from healthy individuals and cell lines (i.e., excluding cancer-predisposition syndromes). Lower DNA Integrity Number corresponds to more fragmented DNA. d, Proposed mechanism for the ssDNA T>A artifact calls in fragmented DNA when performing standard HiDEF-seq v2 (with A-tailing). e, Modifications of the standard HiDEF-seq v2 protocol to eliminate ssDNA T>A artifacts in fragmented DNA. All trials were from the same DNA extraction aliquot (liver from individual 5697). See Methods for details. Rxn, reaction; PNK, polynucleotide kinase; Bst, Bst large fragment; min, minutes. f, ssDNA call spectra, corrected for trinucleotide context opportunities, for a post-mortem liver sample with fragmented DNA from individual 5697 profiled by three of the protocols shown in panel (e): standard HiDEF-seq v2 with A-tailing (top, same spectrum as panel (b)), HiDEF-seq v2 with a Klenow reaction that does not contain dATP nor ddBTP (middle), and HiDEF-seq v2 with a Klenow reaction containing only ddBTP (bottom). The total number of ssDNA calls and total ssDNA call burden (calls per base) are shown. g, Fraction of ssDNA calls that are T>A (corrected for trinucleotide context opportunities) versus the ssDNA T>A burden in post-mortem kidney and liver samples profiled with HiDEF-seq v2 without A-tailing (i.e. Klenow reaction −dATP/+ddBTP). h, Concordant dsDNA mutation burdens in sperm sample SPM-1013 measured by standard HiDEF-seq v2 (i.e., with A-tailing) and HiDEF-seq v2 without A-tailing (i.e. Klenow reaction −dATP/+ddBTP). yo, years old. i, Mutational signature contribution to dsDNA mutations detected in HiDEF-seq v2 primary human tissues from individuals without cancer predisposition. All samples, except blood from a 62 year-old individual (1901), were jointly analyzed with fitting of SBS1 and de novo extraction of one additional signature SBSB. Blood samples of the 62 year-old profiled by HiDEF-seq v2 were analyzed separately (plot shows average signature contributions across 5 blood samples) due to identification of an additional outlier signature SBSC likely associated with this individual's history of end-stage renal disease (see legend of FIG. 9c and Supplementary Table 1 for details). Analysis of samples grouped by tissue type, excluding the 62 year-old blood sample, produced similar results. For de novo extracted signatures (SBSB and SBSC), the cosine similarities to the closest matching COSMIC signatures are shown in parentheses. Sperm, kidney and liver samples from an infant (1443) and 18 year-old (1409), and blood from a 4 year-old (5203) were not included here since their number of mutations are too low for reliable signature extraction. e,h, Error bars: Poisson 95% confidence intervals. f,g, Rxn, reaction.

FIGS. 11a-11c. Comparison of HiDEF-seq and NanoSeq. a, Comparison of HiDEF-seq versus NanoSeq dsDNA mutation spectra for individual 63143. b, Comparison of HiDEF-seq versus NanoSeq ssDNA calls per base, separated by call type. For each call type (i.e., C>A, C>G, etc.), each bar represents a different sample. Samples for each call type, from left to right, are 1105 and 6501 for healthy blood; 63143 for POLE blood; and 1443 for kidney. c, Comparison of HiDEF-seq versus NanoSeq ssDNA call spectra for 6501 (Blood, 43 yo), 63143 (POLE blood), and SPM-1060 (sperm, 49 yo). a-c, yo, years old; mo, months old.

FIGS. 12a-12e. dsDNA mutation burdens and patterns in cancer-predisposition syndromes. a, Fraction of dsDNA mutations in each context. Non-cancer predisposition samples are (left to right): Blood (B) 5203, 1105, 1301, 6501, and 1901; lymphoblastoid cell line (LCL) GM12812; primary fibroblasts GM02036 and GM03348. Cancer predisposition samples are (left-to-right, in the same order and annotated sample types as top-to-bottom cancer predisposition samples in panel (c)): GM16381, GM01629, GM28257, 55838, 58801, 57627, 1400, 1324, 1325, 60603, 59637, 57615, 63143 (LCL), 63143 (B), CC-346-253, CC-388-290, CC-713-555. Affected genes annotated below. Note, GM02036 (asterisk) has a significant increase in C>T mutations with a spectrum matching COSMIC SBS7a (ultraviolet light exposure), likely due to the fibroblasts deriving from sun-exposed skin. b, Representative dsDNA mutation spectra for each affected gene, corrected for trinucleotide context opportunities. Sample IDs are in parentheses. Ages (yo, years old) are listed for blood samples. c, Fraction of dsDNA mutations attributable to de novo extracted dsDNA mutational signatures. Sample genotypes are on the right (hom., homozygous; compound heterozygous variants separated by ‘/’). Cosine similarity to highest matching COSMIC signature is shown in parentheses. For SBSF, the similarity to SBS18 and SBS36 are also shown since these have been previously associated with MUTYH. These MUTYH signatures were not extracted due to the normal mutation burdens of our MUTYH blood samples (see panel d), which is expected at these sample ages and our interrogated base coverage²⁴. Note that SBS40 resembles SBS18 and SBS36 in the C>A spectrum that is enriched in MUTYH syndrome²⁴. Signature extraction was performed for samples of each DNA repair pathway (except XPC separately from ERCC6/ERCC8), while simultaneously fitting COSMIC SBS1 and SBS5 (Methods). Samples are the same top-to-bottom order as left-to-right cancer predisposition samples in panel (a). d, dsDNA mutation burden per base pair (bp) divided by the age of the individual in years (yr) at the time of blood collection, corrected for trinucleotide context opportunities and sensitivity. Only blood samples are shown, since this is the only sample type with ages. Accordingly, there are no nucleotide excision repair syndrome blood samples, so this category is not shown. Non-cancer predisposition blood samples are the same (left-to-right) as in panel (a) (left-to-right). Cancer predisposition blood samples are the same (left-to-right) as blood samples in panel (c) (top-to-bottom). Affected genes annotated below. e, In POLE samples, dsDNA mutations occur more often with AGA>ATA on the non-reference (−) than on the reference (+) strand in genomic loci where the non-reference strand is synthesized more frequently in the leading direction (i.e., positive fork polarity) based on replication timing data (Methods). Random loci are the average of 50 sets of 1,000 random genomic loci with either the sequence AGA or TCT for which there is Repli-seq data at the locus. Reference (+) strand refers to the plus strand of the human reference genome. X-axis is fork polarity, divided into 9 quantile bins from 0 to 1, with higher values corresponding to a greater probability of the non-reference strand being copied in the leading rather than lagging strand direction (Methods). Y-axis for POLE HiDEF-seq samples is the ‘strand ratio’, calculated as the fraction of all AGA>ATA non-reference strand mutations that are in the fork polarity quantile bin divided by the fraction of all AGA>ATA reference strand mutations that are in the fork polarity quantile bin. The ‘strand ratio’ for random genomic loci is calculated as the fraction of all AGA non-reference strand loci that are in the fork polarity quantile bin divided by the fraction of all AGA reference strand loci that are in the fork polarity quantile bin. POLE samples are the same top-to-bottom order in the legend as top-to-bottom POLE samples in (c). Asterisks signify statistical significance in comparison of the POLE 4 sample average (dashed line) to random loci (heteroscedastic two-tailed t.test); p-values left-to-right for asterisks: 3.7·10⁻¹⁷, 0.001, 0.009, 0.02, 0.003. An analysis excluding mutations overlapping genes, to exclude biases due to transcription strand, produced similar results with significant p-values of p=3.1·10⁻¹⁰, 0.003, and 0.04 for quantiles 0-0.1, 0.1-0.2, and 0.6-0.7, respectively, but this analysis has reduced power due to the 55% reduction in the number of mutations analyzed. Note, ssDNA mismatches are not plotted here due to the small number of ssDNA mismatches per fork polarity quantile bin, and instead are plotted in FIG. 2g for negative versus positive fork polarity. a-e, See additional samples details in Supplementary Tables 1-2. e, Error bars, standard deviation.

FIGS. 13a-13f. Burdens and kinetic profiles of ssDNA C>T calls for interpulse duration and after randomization of molecule labels. a, Fraction of ssDNA calls that are C>T (corrected for trinucleotide context opportunities) across all HiDEF-seq v2 samples from healthy individuals and cell lines (i.e., excluding cancer-predisposition syndromes), versus the ssDNA C>T burden. Data shown for kidney and liver samples profiled with HiDEF-seq v2 without A-tailing. Sperm consistently have the highest fraction of ssDNA calls that are C>T. LCL, lymphoblastoid cell line. b, Average ratio of interpulse duration of C>T sequencing calls and 30 base pairs flanking the call, of each molecule with the call relative to molecules aligning to the same locus without the call. Data includes the same samples and calls as in FIG. 3g. c, Average ratio of pulse width (left column) and interpulse duration (right column) after randomizing labels of molecules with and without the calls, for the same samples and calls as in FIG. 3g. d, dsDNA mutation and ssDNA call burdens of heat-treated blood DNA in an additional experiment testing the effect of different buffers and different DNA extraction methods (Puregene alcohol precipitation, orange underline; versus MagAttract with magnetic beads, all other samples). MgAc, magnesium acetate; MgCl₂, magnesium chloride; KCl, potassium chloride; KAc, potassium acetate; Alb, albumin; Tris buffer is Tris-HCl except for the MgAc/KAc/Alb that is Tris-Acetate (see Supplementary Table 1 for concentrations). The percentage of ssDNA sequencing calls that are C>T are annotated above each sample. The cosine similarity to the COSMIC dsDNA signature SBS30 is annotated below each sample, after collapsing ssDNA calls to 96 central pyrimidine trinucleotide contexts and correcting for trinucleotide context opportunities, except for the no-heat treatment samples that do not have sufficient C>T calls. e, SBS30ss* signature (reproduced from FIG. 3e) compared to spectra of ssDNA calls after 72 C heat damage of blood DNA for 6 hours (h) in only 10 mM Tris buffer (n=10,852 calls) or only water (n=2,751 calls). Spectra are plotted after correcting for trinucleotide context opportunities. Below are the odds ratios of the Tris-only and water-only samples compared to SBS30ss* at C>T contexts. f, Heat map of average pulse width ratios for ssDNA and dsDNA C>T calls for positions −1 to +6, for blood DNA samples heated at 72 C for 6 hours in different buffers or water, and for additional samples for comparison. Kinetic profiles of ssDNA C>T calls across the different buffers are similar, and unbiased clustering (dendrogram) separates them from dsDNA calls and from kinetic profiles after randomizing labels of molecules with and without the calls. dsDNA ‘Blood, heat’: blood DNA heat-treated at 56 C and 72 C (both 3 h and 6 h for each); dsDNA ‘Blood’: 4 samples, not heat treated. dsDNA ‘Kidney and liver’: 10 samples, not heat treated. b,c, Error bars, standard error of the mean. d, Error bars, Poisson 95% confidence intervals.

FIGS. 14a-14f. ssDNA call burdens and patterns in healthy tissues. a,b ssDNA C>T (a) and non-C>T (b) call burdens versus age across all HiDEF-seq v2 samples from healthy individuals (primary tissues). Dashed lines: weighted least-squares linear regression, with a 95% confidence interval (shaded ribbon) shown for the statistically significant association for liver. P-values for the regression for liver versus age are 0.0008 and 0.002 for C>T calls, without and with including post-mortem interval (PMI) as a covariate in a multiple linear regression, respectively. P-values for the regression for liver versus age are 0.0003 and 0.002 for non-C>T calls, without and with including post-mortem interval (PMI) as a covariate in a multiple linear regression, respectively. c, ssDNA call burden versus PMI for liver and kidney samples. Dashed lines: weighted least-squares linear regression. The regressions are not statistically significant neither for all calls, nor for C>T or non-C>T calls. d,e, Fraction of ssDNA calls (d) and dsDNA mutations (e) in each context for all HiDEF-seq samples from healthy individuals and cell lines (i.e., excluding cancer-predisposition syndromes). The number of calls for each sample are listed above. Note, context fractions for samples with low call counts are less reliable. Samples (left to right) are: Sperm: SPM-1013, SPM-1002, SPM-1004, SPM-1020, SPM-1060; Liver: 1443, 1409, 1104, 5697, 5840; Kidney: 1443, 1409, 1104, 5697, 5840; Blood: 5203, 1105, 1301, 6501, 1901×5 replicates; Neurons: 5344, 6371; Fibroblasts: GM02036, GM03348; Lymphoblastoid cell line (LCL): GM12812. Note, GM02036 (asterisk) has a significant increase in C>T mutations with a spectrum matching COSMIC SBS7a (ultraviolet light exposure), likely due to the fibroblasts deriving from sun-exposed skin. f, ssDNA call spectra after pooling calls of samples from healthy individuals and cell lines, separately for each tissue, corrected for trinucleotide context opportunities. The corresponding figure for blood is also shown in FIG. 3a. a-c, Error bars, Poisson 95% confidence intervals.

FIG. 15a-15b. Similarity between SBS30ss* and mitochondrial genome heavy strand A>G dsDNA mutations, and mitochondrial ssDNA call burdens. a, SBS30ss* (cytosine deamination) is collapsed to 96 central pyrimidine trinucleotide contexts and compared to mitochondria heavy strand A>G dsDNA mutations, for different sample sets: (i) HiDEF-seq (v2) liver and kidney samples, including liver samples from which mitochondria were enriched (same set of samples in FIG. 5); (ii) 5697 purified mitochondria sample only (plot includes 81% of the mutations in (i)); (iii) Sample set (i), excluding the 5697 purified mitochondria sample (plot includes 19% of the mutations in (i)). Note, the contexts of SBS30ss* are matched with the reverse complement flanking base contexts of mitochondria heavy strand A>G mutations. The number of dsDNA A>G mutations is indicated. b, Spectrum of ssDNA calls combined from liver and kidney samples as well as liver samples from which mitochondria were enriched, excluding bulk (i.e., non-mitochondria enriched) samples profiled by HiDEF-seq v2 with A-tailing. This corresponds to the set of samples profiled in FIGS. 5a-d.

FIGS. 16a-16c. a, Histogram of consensus sequence lengths (i.e., molecule sizes) for: HiDEF-seq standard fragment size (n=51 samples, median length 1.7 kb), HiDEF-seq large fragment size (n=2 samples, median length 4.2 kb), and standard PacBio (HiFi) (n=10 samples, median length 18.3 kb). Histogram lines show average across samples for each bin. Bin values of each sample type are normalized to the bin with the peak molecule count. b, Histogram as in panel (a) of the number of passes per strand (bin width of 5 passes). Average of medians across samples of sample type: 32.0 (standard fragment size), 15.2 (large fragment size), and 5.7 (HiFi) passes per strand. c dsDNA mutation (left) and ssDNA (right) call burdens of two sperm samples profiled by both standard and large fragment size HiDEF-seq. Error bars, Poisson 95% confidence intervals.

### DETAILED DESCRIPTION

Unless defined otherwise herein, all technical and scientific terms used in this disclosure have the same meaning as commonly understood by one of ordinary skill in the art to which this disclosure pertains.

Unless defined otherwise herein, all technical and scientific terms used in this disclosure have the same meaning as commonly understood by one of ordinary skill in the art to which this disclosure pertains.

Every numerical range given throughout this specification includes its upper and lower values, as well as every narrower numerical range that falls within it, as if such narrower numerical ranges were all expressly written herein.

Any database entry reference, such as reference to a sequence database, incorporated herein the sequence associated with the database entry as it exists on the effective filing date of this application or patent.

The disclosure includes all polynucleotide sequences described herein expressly and by reference, and every polynucleotide sequence referred to herein includes its complementary sequence, and its reverse complement. All segments of polynucleotides from 10 nucleotides to the entire length of the polynucleotides, inclusive, and including numbers and ranges of numbers there between are included. All nucleotide sequences associated with any database accession numbers are incorporated herein by reference as they exist in the database as of the date of the filing of this application or patent. The disclosure includes all polynucleotide sequences described herein expressly or by reference that are between 80.0% and 99.9% identical to the described sequences.

Any one or combination of components and process steps can be omitted from the claims. The disclosure includes all steps and reagents, and all combinations of steps reagents, described herein, and as depicted on the accompanying figures. The described steps may be performed as described, including but not necessarily sequentially. Any described reagent(s) and step(s) may be excluded from the claims of this disclosure. As such, the described reagents, steps, and systems of this disclosure may comprise or consist of any one or combination of said reagents and steps. The disclosure also includes all periods of time, and all reaction conditions, all sample preparation methods, and all temperatures described herein.

As used in the specification and the appended claims, the singular forms “a” “and” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about” or “approximately” it will be understood that the particular value forms another embodiment. The term “about” and “approximately” in relation to a numerical value encompasses variations of +/−10%, +/−5%, or +/−1%.

The disclosure provides methods, compositions and kits for use in polynucleotide sequencing. In some examples the disclosure provides for reducing artifacts that were previously not observed using prior approaches to DNA sequencing. Without intending to be constrained by any particular theory it is considered that the disclosure provides the first ultra-high fidelity single-molecule sequencing. In a non-limiting example, ultra-high fidelity provides for an error rate of less than 1 in 1 million to one billion bases, inclusive, and including all numbers and ranges of numbers there between, for single nucleotide mutations.

In embodiments, the disclosure provides a method for determining the sequence of DNA from a plurality of nucleated cells without amplification of the DNA prior to the sequencing. This approach comprises:
a) providing a biological sample comprising the plurality of nucleated cells;
b) extracting DNA from the plurality of nucleated cells;
c) fragmenting DNA extracted from the plurality of nucleated cells with either a random fragmentation method followed by exonuclease digestion to produce a plurality of blunt-ended DNA fragments, or using a restriction endonuclease to produce a plurality of blunt-ended DNA fragments;
d) exposing the plurality of circularized DNA molecules to a ligase to repair nicks in the plurality of circularized DNA molecules;
e) incubating the DNA fragments with a 3′-5′ exonuclease deficient polymerase and a mixture of dideoxyCTP, dideoxyGTP, and dideoxyTTP to block residual nicks, and optionally including deoxyATP to perform A-tailing;
f) circularizing the plurality of DNA fragments with hairpin adapters ligated to both ends of the DNA fragments to obtain a plurality of circularized DNA molecules;
g) sequencing each of the circularized DNA molecules individually using multiple sequencing passes for each of the circularized DNA molecules; and
h) determining the DNA sequence of each of the plurality of circularized DNA molecules, based on the sequences of each individual DNA molecule's sequencing passes.

In some examples the method provides for separately determining the DNA sequence of each strand of a double stranded DNA molecule.

In some examples the sequence of the double strand DNA molecule comprises at least one nucleotide change that is present in one of the strands together with a complementary nucleotide change in the complementary strand (i.e. double strand mutation).

In some examples the sequence of the double strand DNA molecule comprises at least one nucleotide change that is present on only one of the two strands (i.e. single strand nucleotide change, due to either a single strand nucleotide mismatch or single strand nucleotide damage).

In some examples, the determined DNA sequences comprise no more than one double strand mutation for each 1 million nucleotides of determined DNA sequence base pairs, relative to a reference sequence.

In some examples the determined DNA sequences comprise no more than one single strand nucleotide change for each 1 million nucleotides of determined DNA sequence bases.

In embodiments, a described method may be adapted for use with an existing system, such as so-called “HiFi” sequencing systems as described in Accurate circular consensus long-read sequencing improves variant detection and assembly of a human genome. Nature Biotechnology, 37, 1155-1162, from which the description is incorporated herein by reference. Generally, HiFi sequencing uses a combination of advanced sequencing chemistry and computational methods to achieve higher accuracy and longer read lengths compared to traditional sequencing methods. Thus, a method of the disclosure may be adapted for use with systems currently offered in connection with the tradename PACBIO.

In embodiments, a described method may be adapted for use with so-called Oxford Nanopore sequencing.

In embodiments a described method may be used as a complement to so-called duplex sequencing, such as that offered by TWINSTRAND BIO.

In embodiments a described method may be combined with or used as a complement to existing sequencing technologies as discussed above, which may further include Sanger sequencing and next-generation sequencing (NGS) methods, such as those offered under the tradename ILLUMINA.

As discussed above, in embodiments nucleotide changes that appear at least initially only in a single strand of DNA are determined. In embodiments, mutations that appear at the same location in both strands of DNA are determined. In embodiments, the disclosure provides for quantification of the burden of mosaic mismatches and mutations, i.e., the number of mismatches and mutations per the number of DNA bases and base pairs sequenced, respectively. In embodiments the disclosure provides for determining the burdens and patterns (sequence contexts) of single-strand mismatches and single-strand damage (single-strand DNA calls, ssDNA calls), double-strand mutations (double-strand DNA mutations, dsDNA mutations) in healthy tissues such as sperm, in individuals with cancer-predisposition syndromes such as bi-allelic mismatch repair deficiency and defects in replicative polymerase proofreading, and combinations thereof.

The source of DNA sequenced as provided in this disclosure is not particularly limited. In embodiments, the DNA is from a prokaryotic source. In embodiments, the DNA is from a viral source. In embodiments, the DNA is from a eukaryotic source. In embodiments the eukaryote is a multicellular eukaryote, such as an animal, plant, or fungus. In embodiments the DNA is from a mammal. In an embodiment the DNA is from a human. In embodiments, the DNA is from a propagated cell line. In embodiments, cells from which DNA that is sequenced according to this disclosure are diploid cells. In embodiments, cells from which DNA that is sequenced according to this disclosure are haploid cells, such as gametes, i.e., sperm.

The type of biological sample is also not particularly limited, as long as it contains any type of cells or isolated organelles that comprise DNA. The biological sample may be a liquid biological sample, such as blood, sputum, semen, lacrimal secretions, urine, cerebrospinal fluid, tumors, and the like. The biological sample may be a solid or semi-solid sample, such as a tissue biopsy, tumor tissue, or a hair sample. The sample may comprise disaggregated cells from a tissue, or cells obtained from an in vitro cell culture. The DNA sequence that is determined may be from a nucleus, an organelle, or may be cytoplasmic. In some embodiments, a post-mortem biological sample may be used.

The disclosure provides different options as further described herein that relate at least in part to the quality of the DNA that is sequenced. For example, for a sample that is suspected of having DNA that has been degraded, the disclosure includes performing a described method without A-tailing. A non-limiting example of a sample that can be suspected of having DNA that is degraded is a post-mortem sample. Lower quality DNA may include double stranded breaks and thus may be nicked and/or fragmented. This is in contrast to freshly obtained samples, and samples that are kept under conditions which inhibit DNA degradation, which typically do not have DNA nicks or fragmented DNA. For samples that are suspected to comprise DNA that is not degraded, A-tailing may be performed.

In embodiments, the described method is used to produce DNA sequences that can be compared to one or more reference sequences. This comparison can be used, for instance, to quantify or estimate sequencing fidelity. For multicellular organisms, a suitable reference sequence may be the germline genome sequence. The determined sequence can be compared to the reference sequence by filtering using either standard short-read or long-read genome sequencing of the same individual. In one embodiment, a telomere-to-telomere human reference genome may be used. For cultured cells, a suitable reference may comprise a bulk genome sequence. In embodiments, a described method provides higher fidelity sequencing results compared to a sequence result obtained using a previously available approach, including but not necessarily limited to the aforementioned approaches.

In embodiments, a described method provides improved fidelity relative to previously known sequencing processes, a non-limiting example of which comprises so-called highly accurate long reads, also referred to as HiFi reads, offered under the tradename PacBio®. In embodiments, the disclosure provides for no more than 1 in 1-10 million incorrectly determined bases relative to the sequence of the DNA molecule that was present in a sample, which can in embodiments be determined by comparison to a suitable reference.

In embodiments, the disclosure provides a system, the system comprising: at least one computer hardware processor and optionally one or more databases that may store new or pre-existing genetic sequence information. A system in communication with the database may include at least one non-transitory computer-readable storage medium storing processor-executable instructions that, when executed by a computer hardware processor, cause the computer hardware processor to perform one or more steps and/or algorithms or a computational pipeline as described herein, to generate results that include DNA sequencing results. In embodiments, the disclosure provides a system that includes one or more devices, said devices comprising a polynucleotide sequencing device and a computer. In embodiments, one or more components of the device can be connected to or in communication with digital processor and/or the computer may be running software to interpret polynucleotide sequencing signals, and may function to compare determined polynucleotide sequence to one or more pre- or newly determined reference sequences. In embodiments, a system described herein may operate in a networked environment using logical connections to one or more remote computers. In embodiments, a result obtained using a device, system. or method of this disclosure is fixed in a tangible medium of expression. The result may be communicated to, for example, a health care provider or other data analyst. A sequencing result may be indicative of a predisposition to a condition or the presence of an existing condition, including but not necessarily limited to cancer, exposure to DNA damaging agents, age-related conditions, or a combination thereof. A sequencing result may aid in a health care providers diagnosis of a condition, and may be used to recommend and/or deliver a particular prophylactic or therapeutic agent or other medical intervention.

The disclosure also provides kits for use in a described method. The kit may include one or more sealed containers that contain one or more reagents, such as one or more described enzymes, proteins, primers, buffers, nucleotides and/or modified nucleotides, primers, or combinations thereof. The kits may comprise printed material that provides instructions on use of the kit components, sample preparation, and the like. The kit may include solutions such as buffers. Dry forms of reagents, i.e., a lyophilized/powered or other form that is suitable for reconstitution in a buffer may be provided. The kits may comprise nucleotides that include only dideoxyCTP, dideoxyGTP, and dideoxyTTP. For certain approaches the kits may also include deoxyATP in a solution or configured in a dry form. Any of the described kit components may be provided in a frozen form.

In view of the foregoing discussion the disclosure includes the following description and examples which are intended to illustrate but not limit the disclosure.

### EXAMPLES

### Hairpin Duplex Enhanced Fidelity Sequencing (HiDEF-Seq)

Profiling dsDNA mosaic mutations in human tissues requires single-molecule fidelity of <1 error per 1 billion bases (10⁻⁹), and profiling ssDNA mismatch and damage events would likely require similar or greater fidelity^{17,19,25}. However, no invention prior to the present disclosure, has achieved this fidelity when directly sequencing unamplified single DNA molecules. Pacific Biosciences (PacBio) single-molecule sequencers achieve a fidelity of ˜1 error per 1,000 bases (10⁻³) by sequencing dsDNA molecules that have been topologically circularized with hairpin adapters²⁶. Circularization allows the sequencing polymerase to go around the DNA molecule several times (“passes”), up to its limit of processivity (˜160 to 220 kilobases, kb), termed the polymerase read length²⁶. This is then followed by creation of a single-molecule consensus sequence, which filters the random errors of each pass²⁷. This multi-pass approach has yet to demonstrate ultra-high (10⁻⁹), single-molecule fidelity, because: (1) the fidelity of each pass has a high error rate (higher for insertions and deletions than substitutions), (2) the number of passes for each DNA molecule is low (˜6 passes per strand for ˜13 to 18 kb molecules), so that without accurate computational filtering, sequencing errors are not completely filtered from the molecule's final consensus sequence²⁶, and (3) standard methods of preparing DNA for sequencing introduce artifacts, since library preparation polymerases can replace the original DNA beginning at single-strand nicks and at fragment ends¹⁷. Supporting the feasibility of higher fidelity PacBio sequencing, one study estimated a dsDNA mutation burden of ˜10⁻⁷ in bacterial cells after manual review of mutation calls²⁸, though it did not utilize novel library preparation methods that prevent the latter artifacts¹⁷.

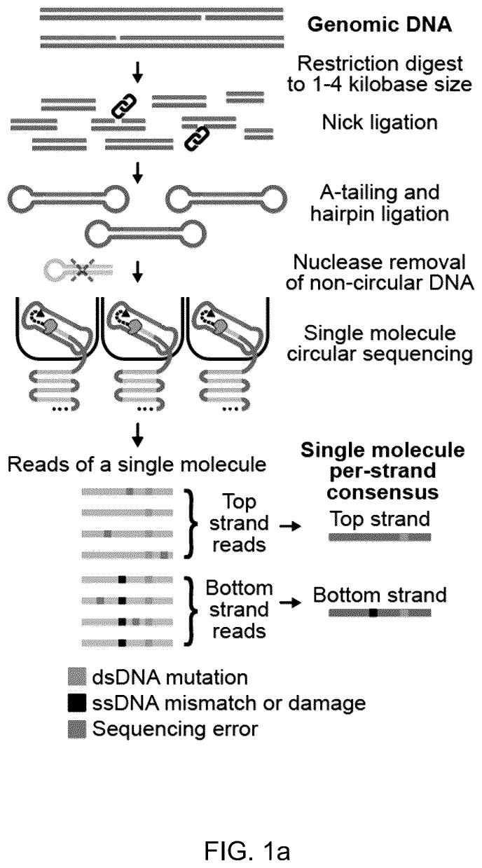

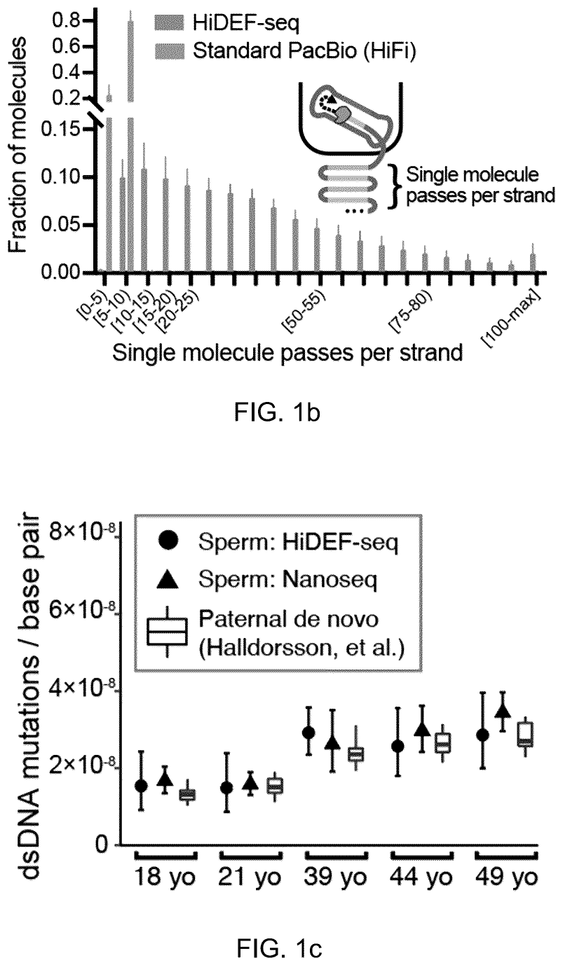

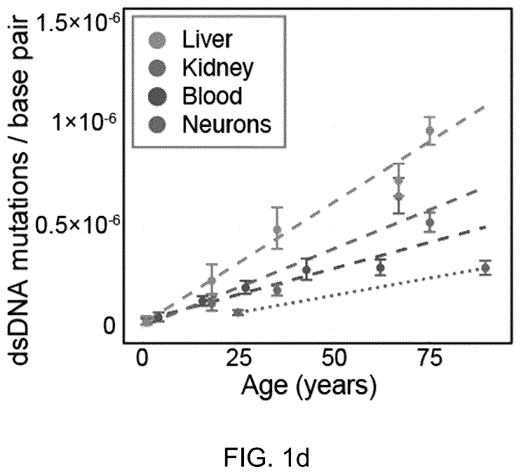

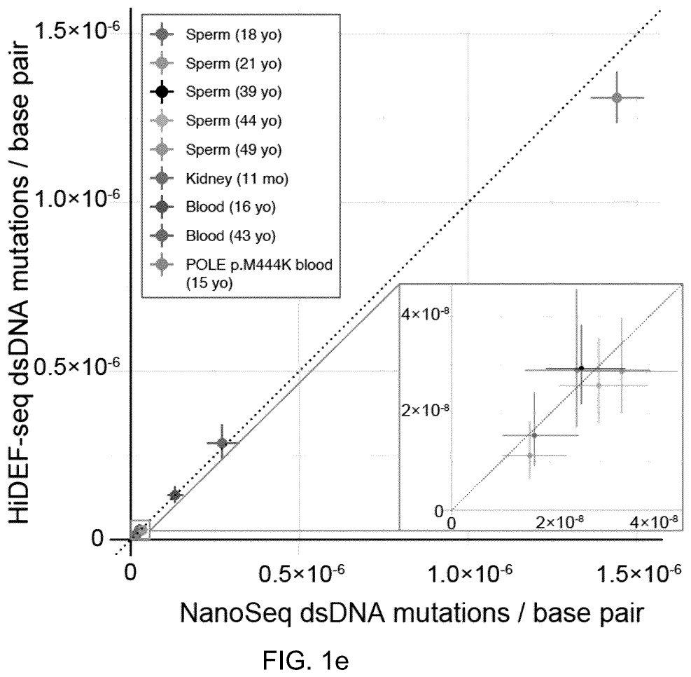

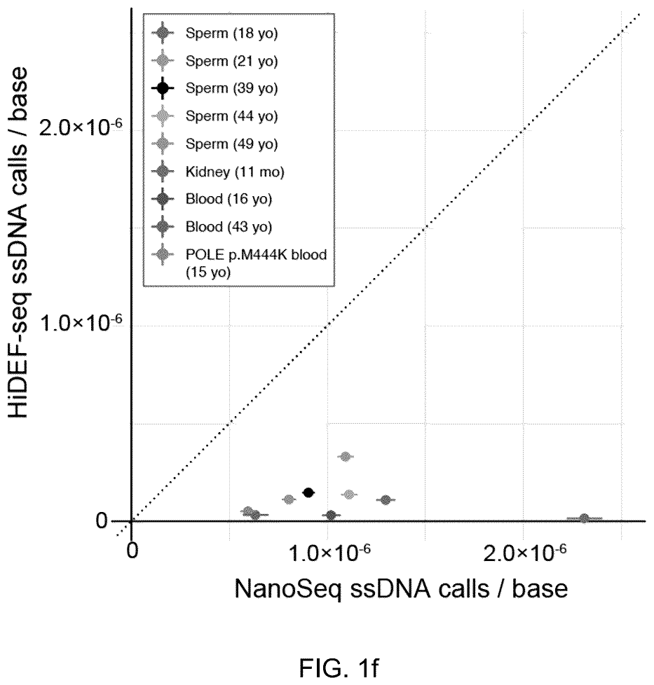

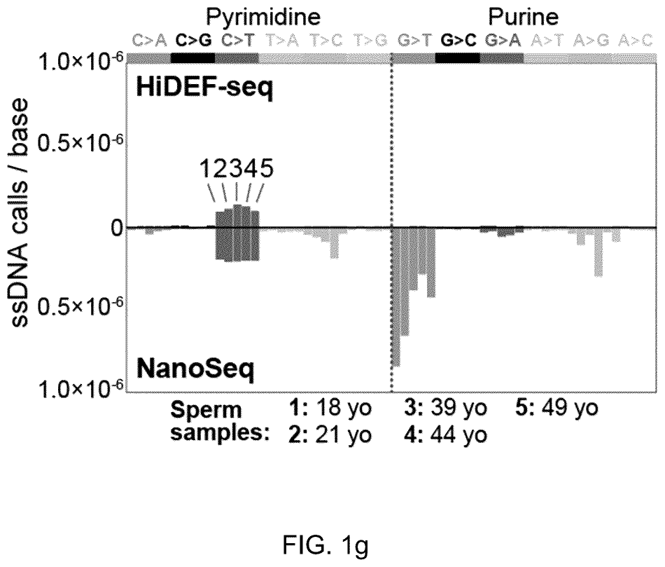

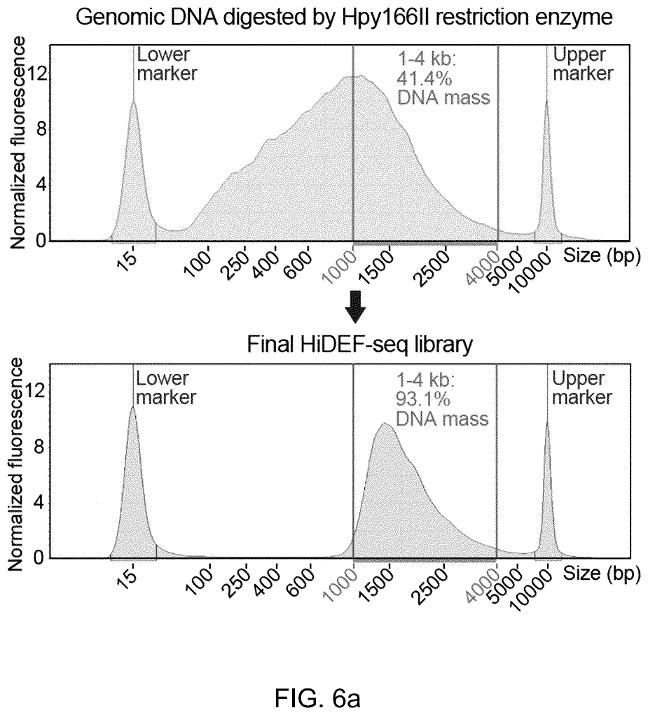

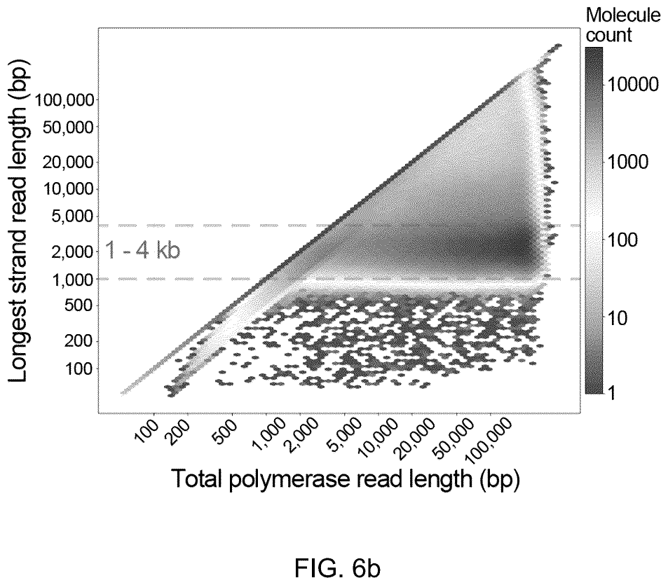

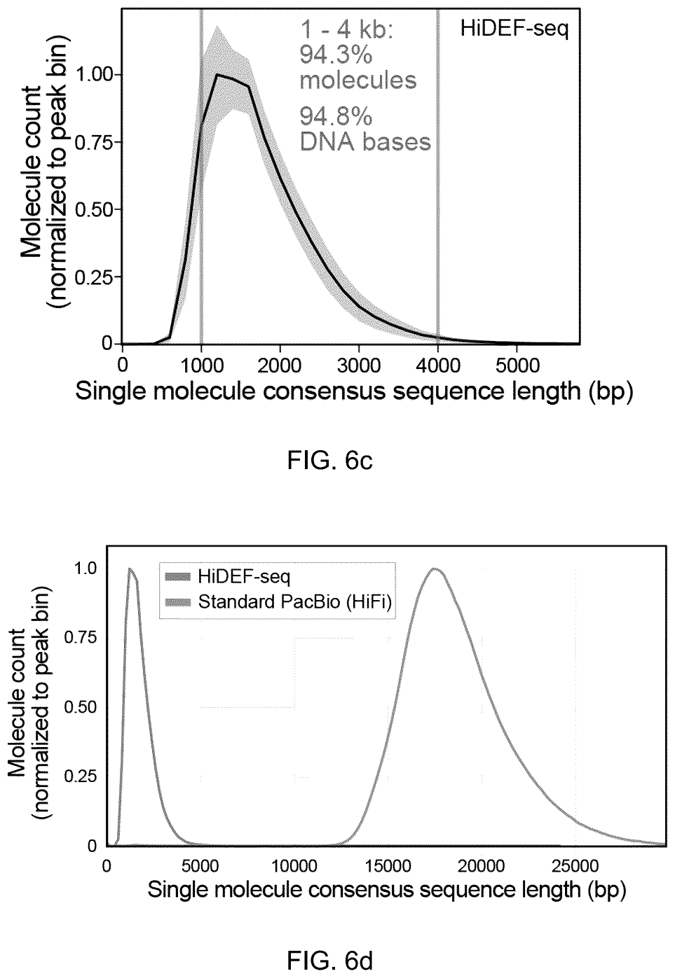

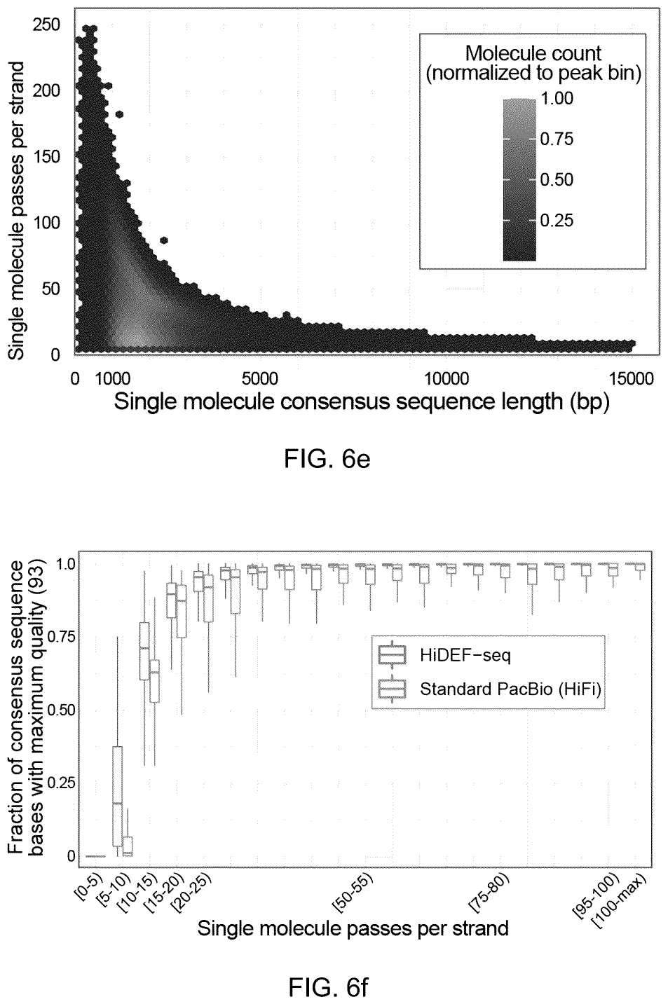

To increase single-molecule sequencing fidelity >1 million-fold relative to standard sequencing, the present disclosure provides an approach referred to as Hairpin Duplex Enhanced Fidelity Sequencing (HiDEF-seq). HiDEF-seq dramatically increases single-molecule sequencing fidelity by increasing the number of passes per molecule, by eliminating in vitro artifacts during library preparation, and by a computational pipeline that avoids analytic artifacts (FIG. 1a and FIGS. 6-9). Given that 6 passes per strand currently achieves an error rate of 10^{-3.26}, and assuming that errors are mostly random in each pass at least for substitutions²⁹, we estimated that at least 3 times as many passes would be required to decrease the error rate to 10⁻⁹. We therefore set a conservative goal of 20 passes per strand for most molecules, which we achieved by fragmenting DNA with a restriction enzyme to a size range of 1 to 4 kilobases (160 kb polymerase read length/[2 strands×20 passes per strand]). We capture approximately 40% of the human genome in this size range (FIG. 6a), which is sufficient for obtaining accurate mosaic mutation burdens and mutational patterns¹³. HiDEF-seq can also also utilize random fragmentation to enable profiling of any genomic region, by treating the fragmented DNA with an exonuclease that removes single-strand overhangs to leave blunt-ended DNA. To minimize in vitro library preparation artifacts, we also utilized the DNA A-tailing approach of NanoSeq, a recently developed method for profiling mosaic dsDNA mutations that performs A-tailing together with non-A dideoxynucleotides to block ssDNA nicks¹³. Sequencing of HiDEF-seq molecules showed the expected distribution of molecule lengths (median 1.7 kb), a median of 32 passes per strand, and 70% of molecules that pass primary data processing with 20 passes per strand, representing a 5.6-fold increase in median passes per strand compared to standard PacBio (HiFi) sequencing (FIG. 1b and FIGS. 6b-e). Further, the fraction of bases in each DNA molecule with the maximum possible consensus sequence base quality (<10⁻⁹ predicted errors per base) increased exponentially with increasing passes, and for molecules with 20 passes per strand, almost all bases (median 99%) reached this maximum predicted consensus sequence quality (FIG. 6f).

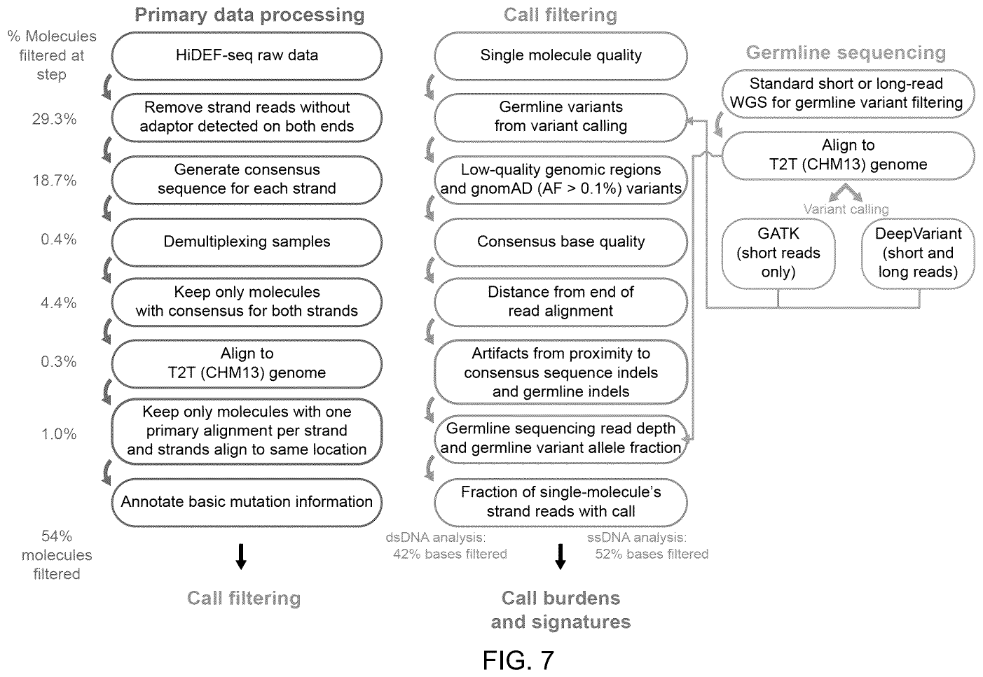

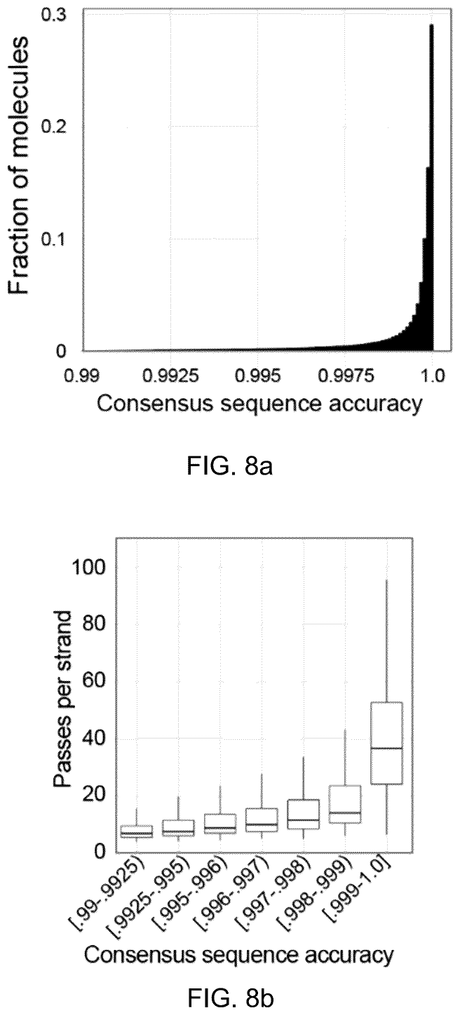

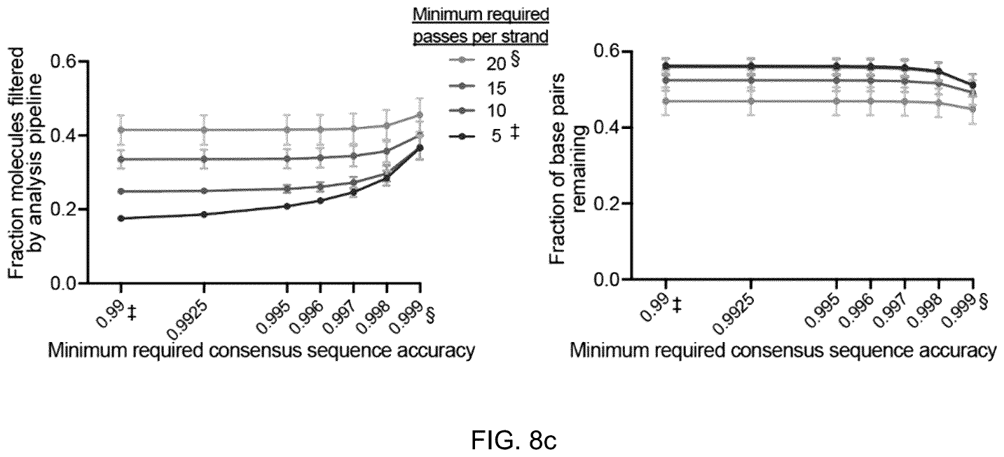

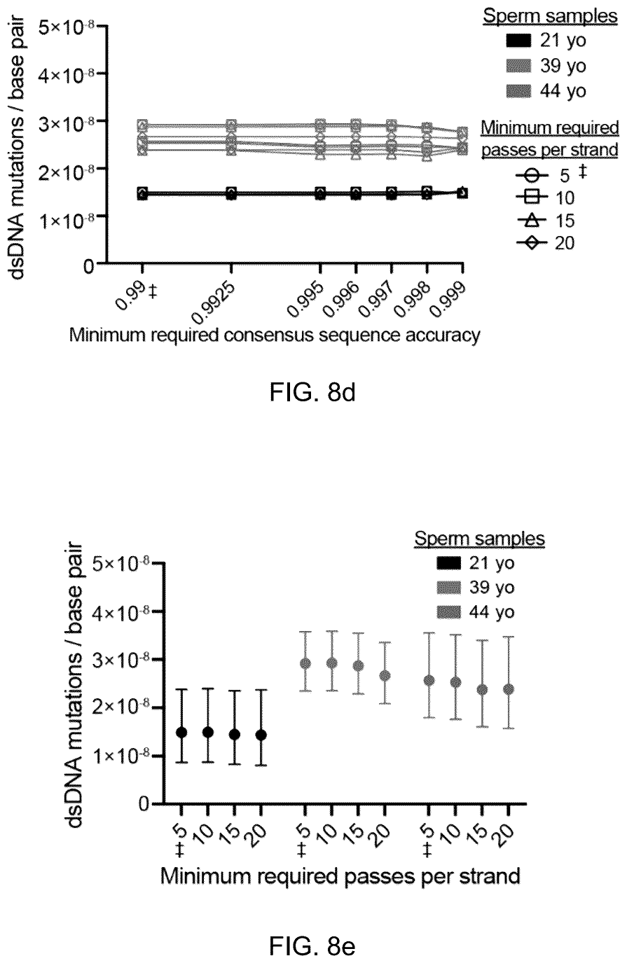

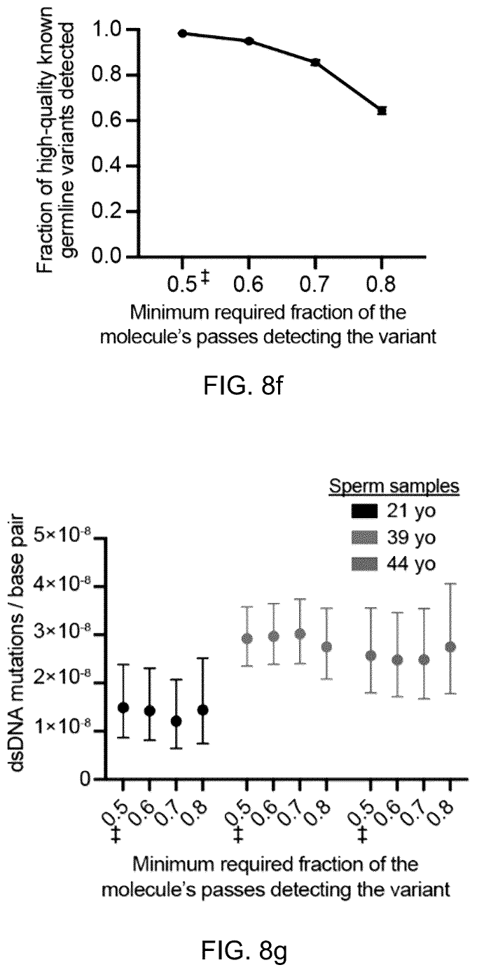

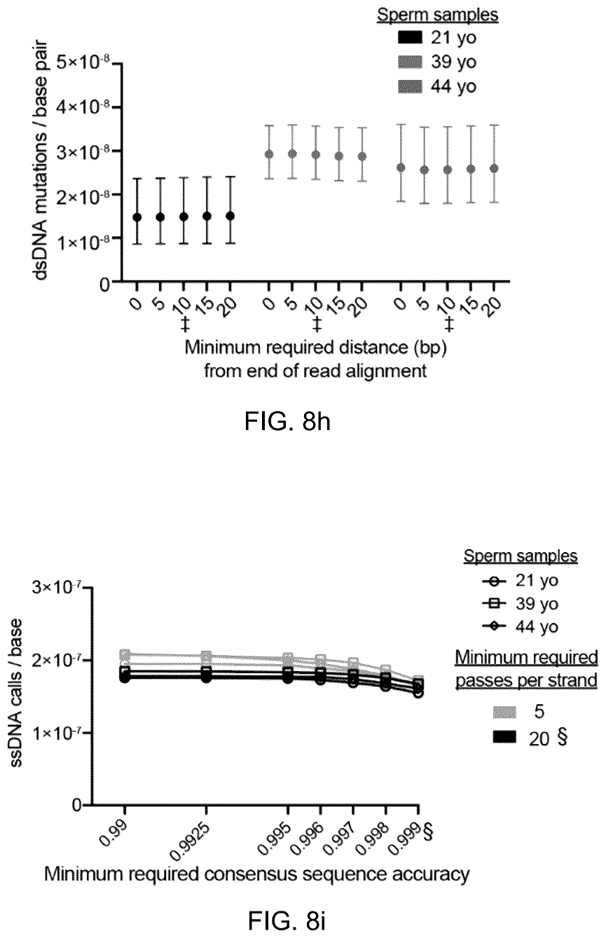

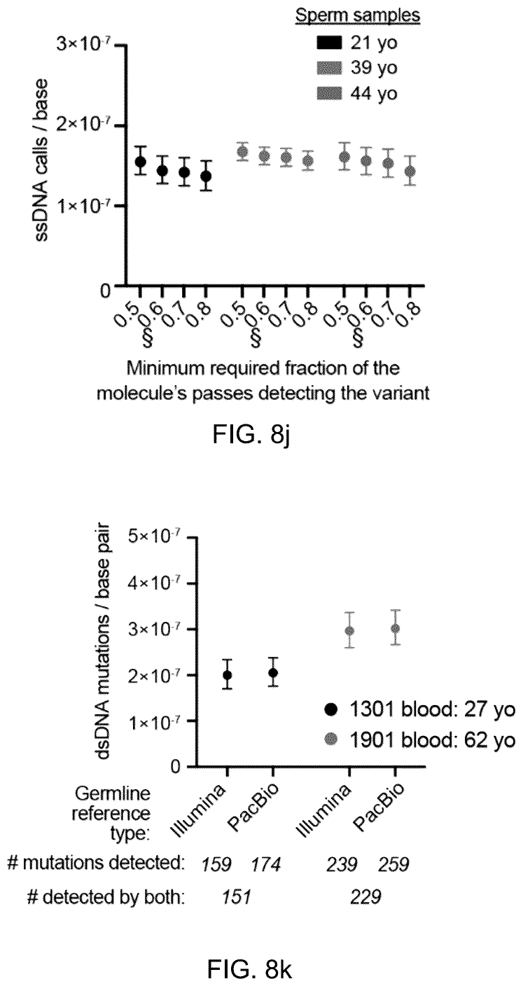

With this predicted sequencing fidelity, computational artifacts due to alignment and reference genome errors³⁰ represent a greater source of error than sequencing error when trying to detect rare mosaic events. The disclosure therefore includes a computational pipeline that utilizes the telomere-to-telomere human reference genome, which was itself constructed using long reads³¹. The pipeline analyzes single base substitutions, since these have an orthogonal error profile to the prevalent insertion and deletion sequencing errors of single-molecule sequencing³², and it analyzes each strand separately and then compares them to distinguish dsDNA from ssDNA events. It further filters low quality DNA molecules and consensus sequence bases, remaining regions of the genome prone to false positives, and germline (i.e. inherited) variants (FIGS. 7,8 and Methods). Germline variants can be filtered using either standard short-read or long-read genome sequencing of the same individual (FIGS. 8k,l).

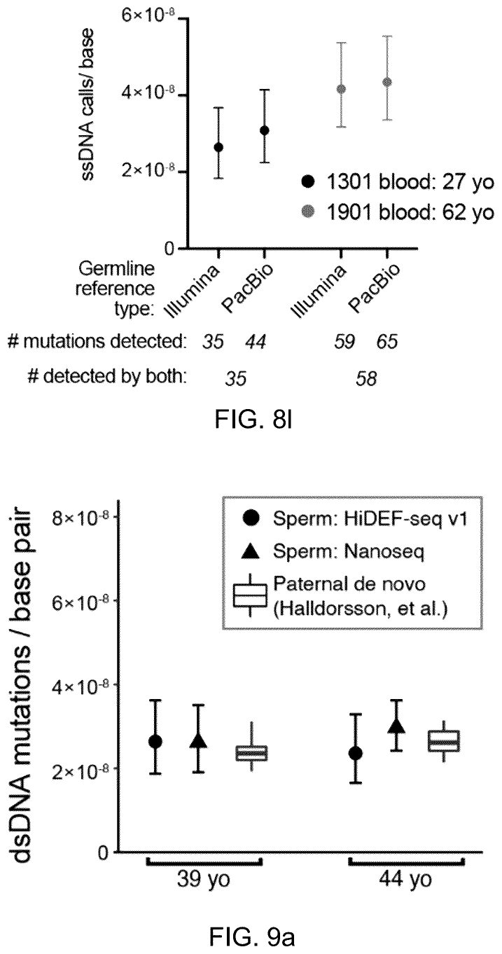

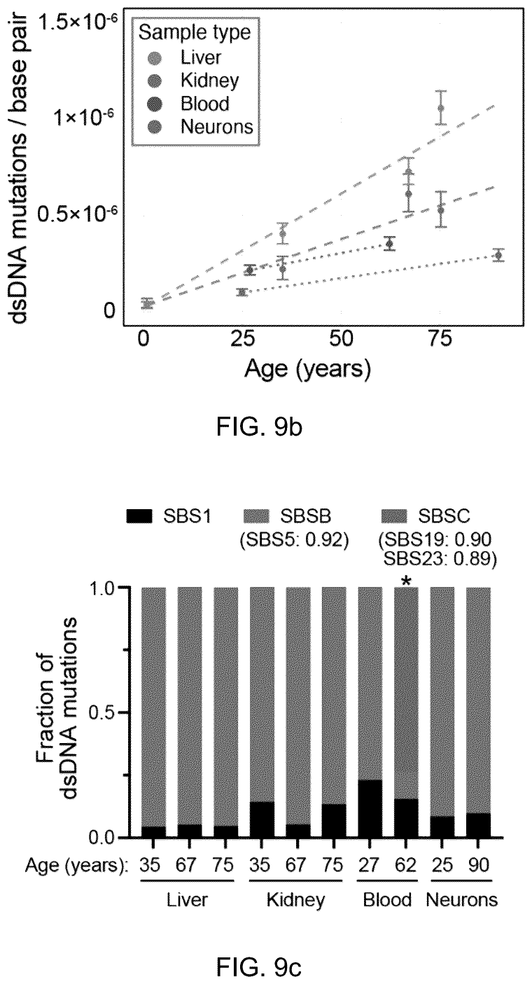

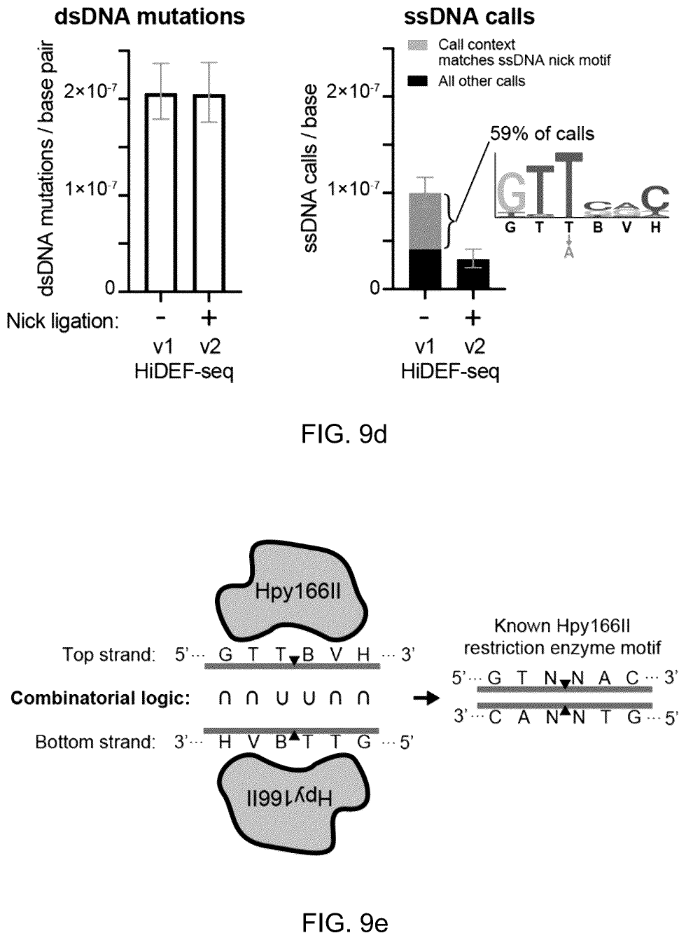

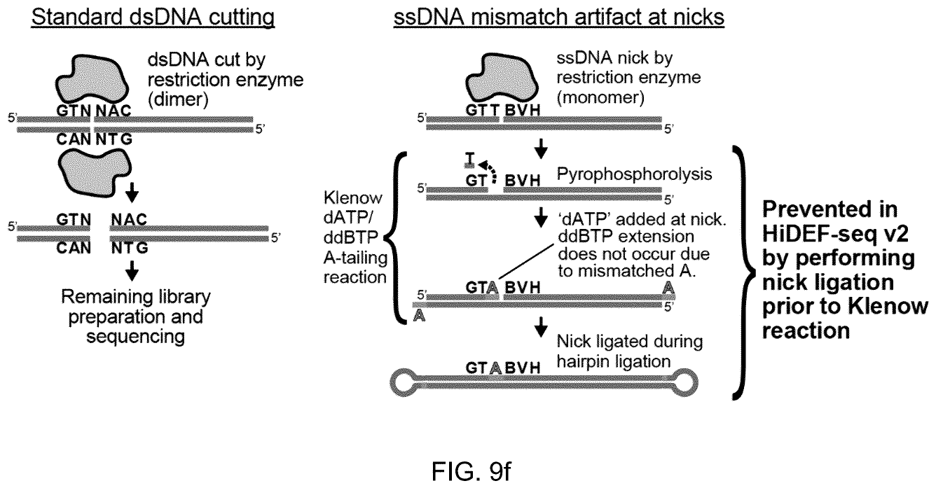

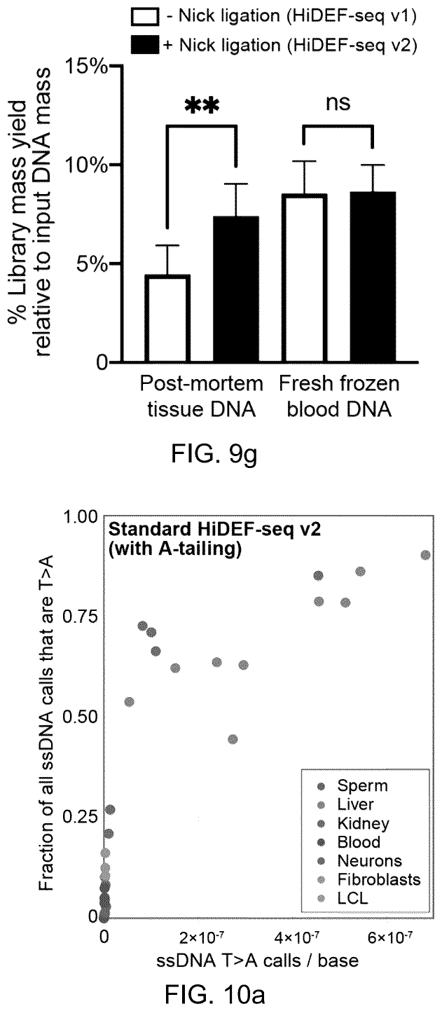

With this first version of HiDEF-seq (v1), we profiled purified human sperm as the most rigorous test of fidelity for detecting dsDNA mosaic mutations-since sperm harbor the lowest dsDNA mutation burden of any readily accessible human cell type. Sperm dsDNA mutation burdens as measured by HiDEF-seq v1 were concordant with NanoSeq profiling¹⁷ that we performed for the same samples and with a prior study of de novo mutations²⁰ (FIGS. 9a). HiDEF-seq also measured the expected dsDNA mutational signatures and increase in dsDNA mutation burden with age in other primary human tissues (kidney, liver, blood, and cerebral cortex neurons) (FIGS. 9b-c)^{15,17} with one outlier blood sample exhibiting a signature similar to COSMIC³³ signatures SBS19 and SBS23 in an individual with a kidney transplant due to end-stage renal disease (FIGS. 9b-c). The latter finding is consistent with a prior report that found SBS23 in renal cell carcinoma of individuals with end-stage renal disease who underwent long-term renal hemodialysis³⁴, suggesting that end-stage renal disease and/or hemodialysis are mutagenic to healthy tissues. Notably, relaxing from a threshold of ≥20 to ≥5 passes per strand, while keeping the filter for maximum consensus sequence base quality, produced concordant results (FIG. 8e). This indicates that the filter for consensus sequence base quality (a measure of the level of support for the consensus sequence provided by the individual passes) is of primary importance, and that with our computational approach, PacBio sequencing can achieve a higher per-pass fidelity for substitutions than estimated by prior studies²⁶. Accordingly, for ultra-high fidelity analysis of dsDNA mutations, we used this lower threshold of 5 passes per strand as this increases the percentage of molecules included in the analysis from 70% to 99.8% (of molecules that pass primary data processing), and it increases the percentage of bases that are interrogated by 11%. We also successfully quantified the dsDNA mutation burden of sperm using HiDEF-seq with larger DNA fragments (median 4.2 kb), which have correspondingly fewer passes per strand (median 15) (Supplementary Note 1). However, for this study, we proceeded with HiDEF-seq with the smaller median 1.7 kb fragments, since a higher threshold of 20 passes per strand was required for ssDNA analysis.

Next, we proceeded to analyze ssDNA calls. ssDNA calls may include not only ssDNA mismatches, but also damaged bases that alter base pairing properties and lead to mis-incorporation of nucleotides by the sequencer polymerase. The latter is potentially advantageous as it would enable high fidelity detection of ssDNA damage. However, whereas dsDNA mutation analysis can take advantage of information in both strands (duplex error correction) and its fidelity can be confirmed using the expected mutation burden of sperm, duplex error correction is not possible for ssDNA calls, and ssDNA mismatch burdens are unknown. Hence, for ssDNA calling we optimized certain analytic parameters by identifying filter thresholds above which ssDNA burden estimates are stable and identifying any patterns that may suggest artifacts (FIGS. 8i-j and Methods).

Upon initial analysis of HiDEF-seq v1's ssDNA calls, we identified that approximately 60% of them were T>A changes at a motif corresponding to half of the restriction enzyme recognition sequence that we use to fragment the DNA, likely because the enzyme normally operates as a dimer while creating rare ssDNA nicks as a monomer (FIGS. 9d-f). In a larger set of sperm samples, this improved version of HiDEF-seq (v2) again measured the expected dsDNA mutation burden (FIG. 1c)²⁰, with a fidelity for dsDNA mutations (estimated as the probability of complementary single-strand mismatches occurring at the same position; Methods) of <1 error per 7·10¹⁶ base pairs and <1 error per 10¹⁷ base pairs with ≥5 and ≥20 passes per strand, respectively.

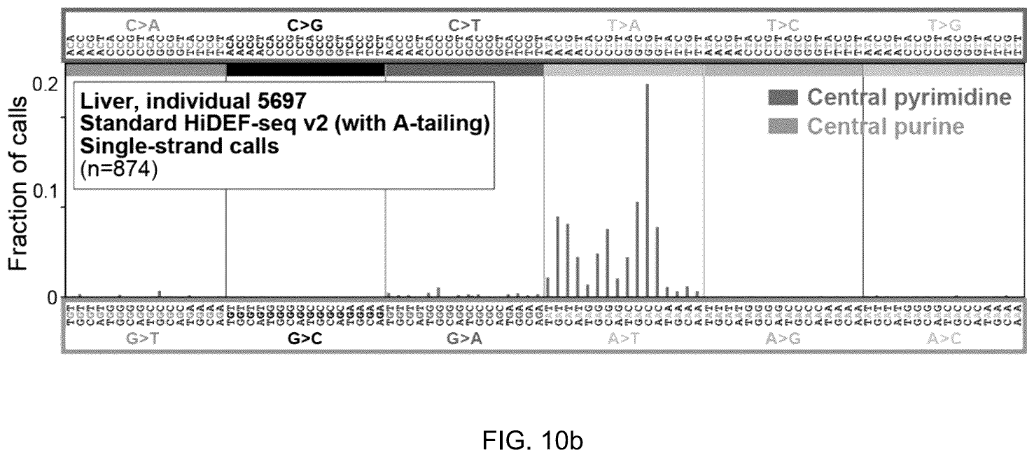

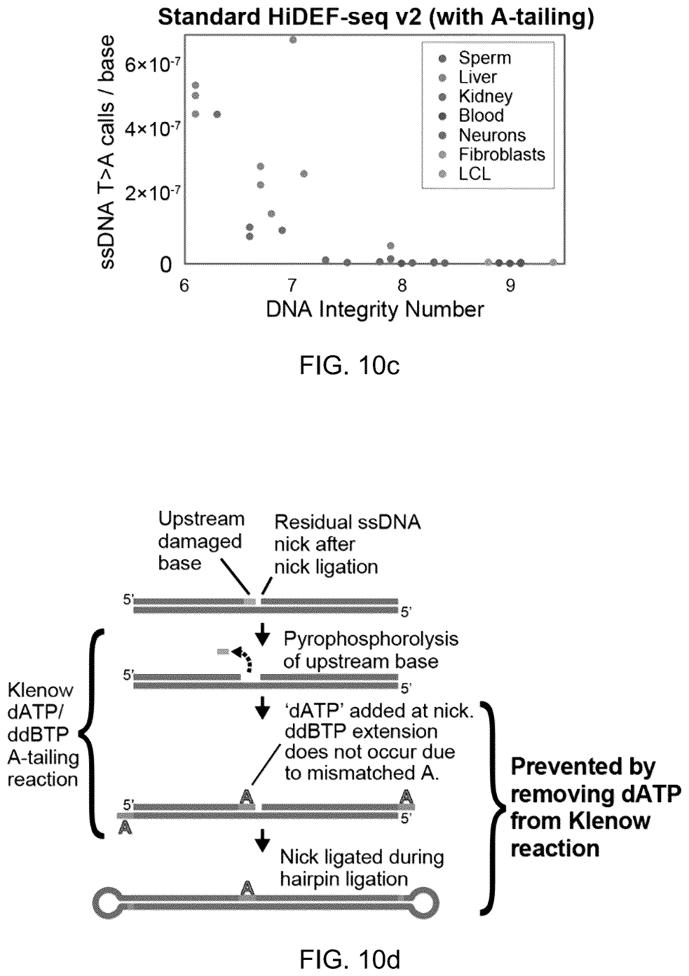

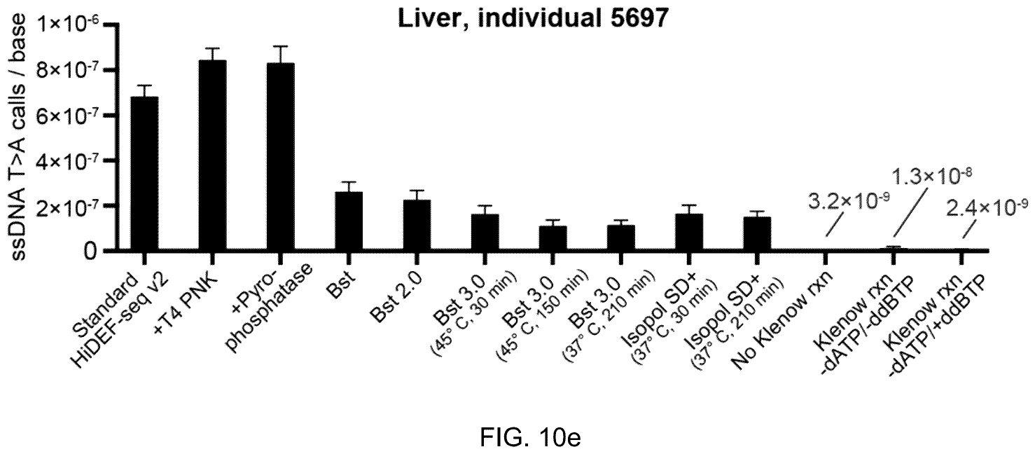

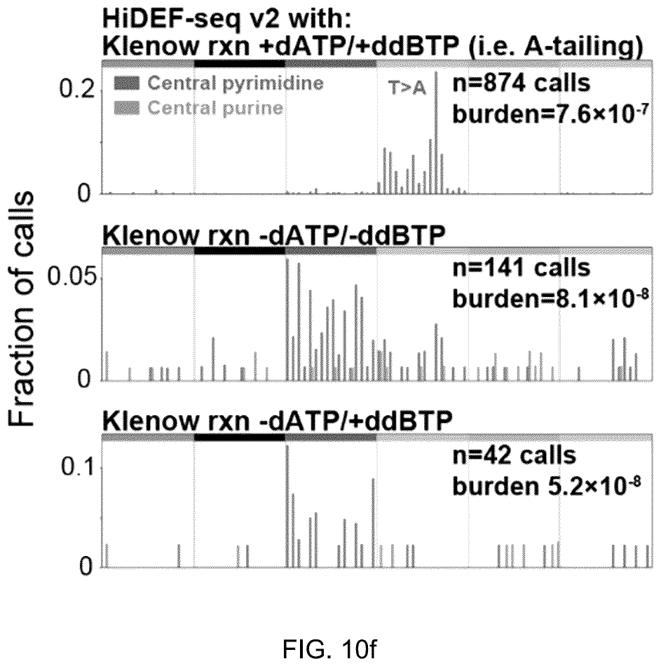

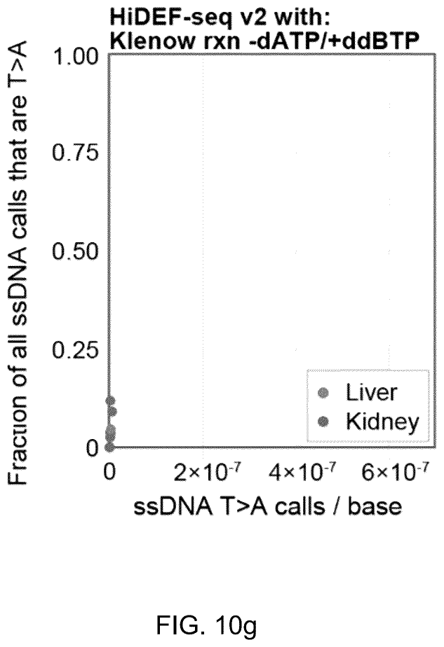

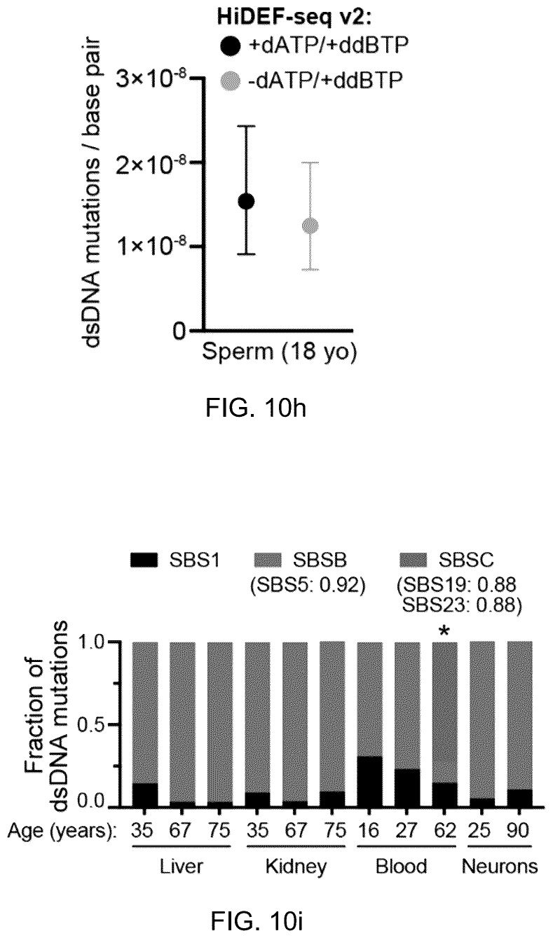

Across all sample types profiled in this disclosure with HiDEF-seq v2 (kidney, liver, blood, sperm, cerebral cortex neurons, primary fibroblasts, and cell lines), we found that post-mortem kidney and liver samples still exhibited a significant burden of T>A ssDNA calls (average 67% of calls and average 1 call per 3.5 million bases) that correlated with the extent of post-mortem DNA fragmentation (FIGS. 10a-c). These presumed artifactual ssDNA T>A calls did not correspond to any recognizable sequence motif and likely occurred due to post-mortem ssDNA nicks that HiDEF-seq v2's nick ligation step was unable to seal due to an upstream damaged DNA base at the nick site; then, during A-tailing, this damaged base may still be removed by a pyrophosphorolysis mechanism and then replaced by a mismatched deoxyadenosine (FIG. 10d). We trialed a variety of approaches to remove these T>A ssDNA artifacts so that HiDEF-seq v2 could also be used to profile post-mortem tissues with fragmented DNA. This included treating the DNA with polynucleotide kinase to attempt to improve nick ligation, performing A-tailing in the presence of pyrophosphatase to attempt to decrease pyrophosphorolysis, and performing A-tailing with different polymerases and reaction conditions to attempt to reduce pyrophosphorolysis and improve extension from the mismatched A so that a subsequent dideoxynucelotide can be incorporated (Methods). While some of these approaches significantly decreased these T>A ssDNA artifacts, they did not entirely eliminate them (FIG. 10e). Instead, we discovered that altogether removing A-tailing from the protocol completely eliminated these T>A ssDNA artifacts, and further, that retaining a polymerase nick extension step with non-A dideoxynucleotides removes low-level random-context artifactual ssDNA calls to produce final ssDNA call patterns similar to non-post mortem tissues and without any discernible artifacts (FIGS. 10e-g). We also profiled sperm with this non-A-tailing HiDEF-seq v2 protocol and confirmed its dsDNA mosaic mutation fidelity (FIG. 10h). The only disadvantage to removing A-tailing from HiDEF-seq v2 is a requirement for approximately double the amount of input DNA due to the lower efficiency of subsequent blunt adapter ligation (Methods). We therefore utilized standard HiDEF-seq v2 for nearly all samples, except for post-mortem kidney and post-mortem liver samples for which we utilized HiDEF-seq v2 without A-tailing (Supplementary Table 1).

Similar to HiDEF-seq v1, profiling of primary human tissues (kidney, liver, blood, and cerebral cortex neurons) with HiDEF-seq v2 exhibited the expected dsDNA mutational signatures and linear increase in dsDNA mutation burden with age^{15,17} (FIG. 1d and FIG. 10i). For simplicity, unless otherwise specified we subsequently refer to HiDEF-seq v2 (both with and without A-tailing) as HiDEF-seq.

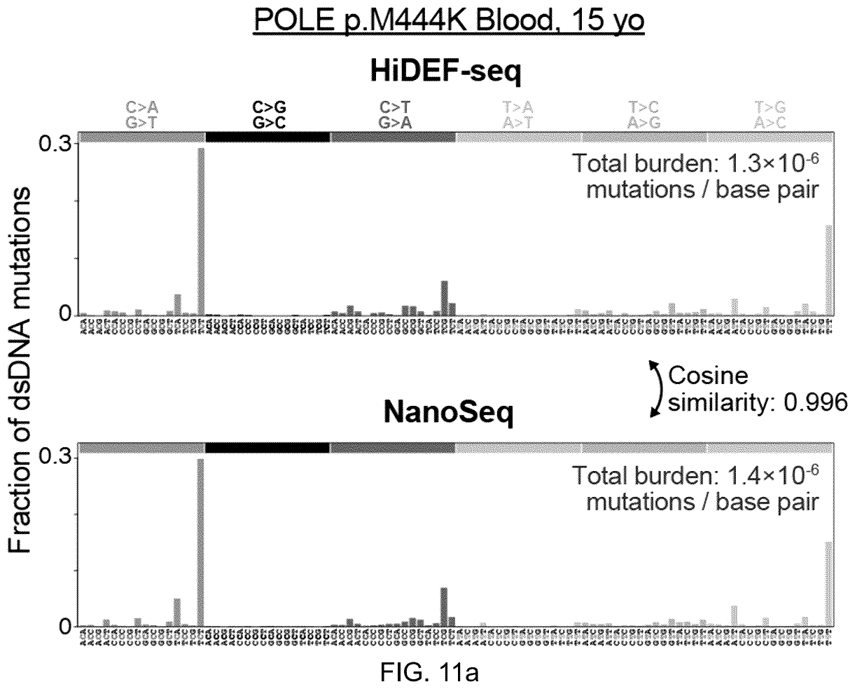

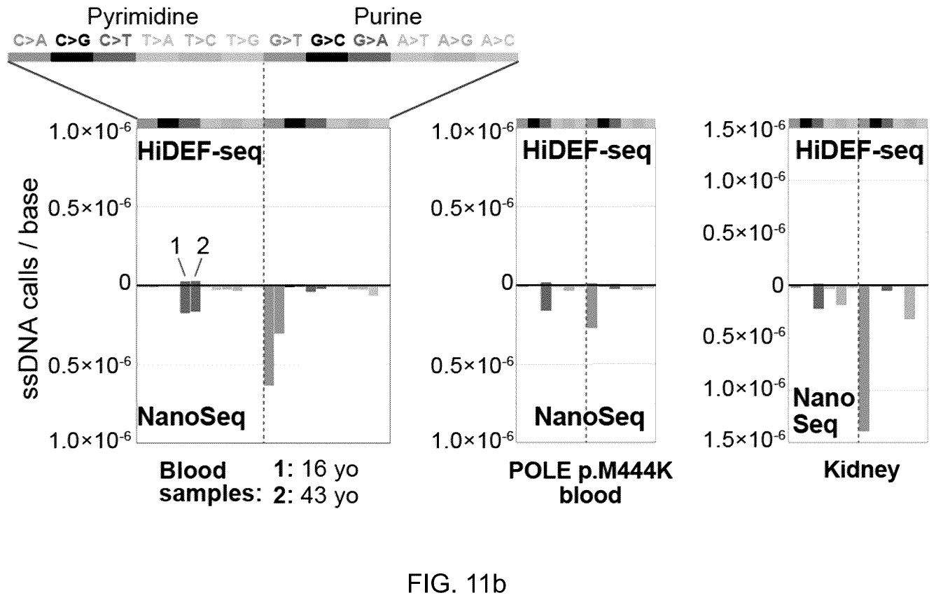

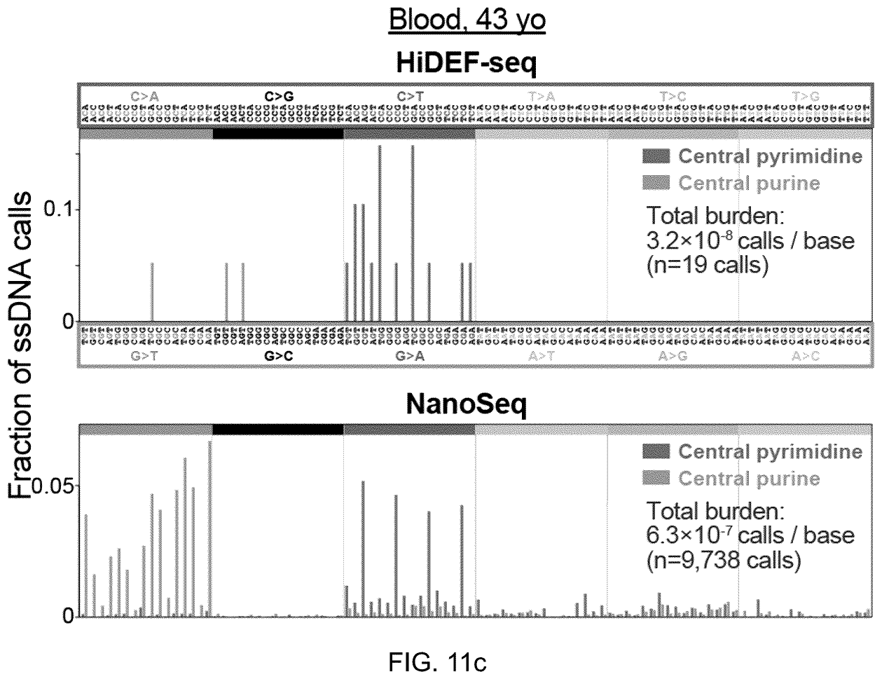

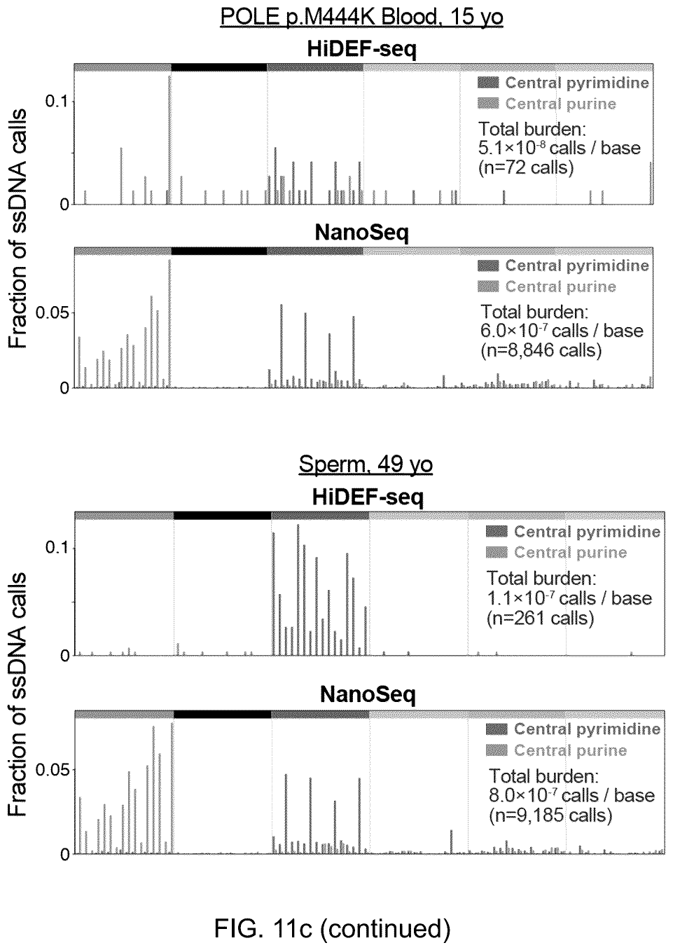

To compare ssDNA calls between HiDEF-seq and Nanoseq, we profiled 9 samples with both methods. While HiDEF-seq and NanoSeq dsDNA mutation burdens and patterns were concordant, HiDEF-seq measured on average 28-fold fewer ssDNA call burdens than Nanoseq, with distinct patterns (FIGS. 1e-g and FIGS. 11a-c). This suggests that NanoSeq's ssDNA calls are largely artifactual as suggested by its developers¹³. The HiDEF-seq ssDNA burden measured for cerebral cortex neurons was also ˜13-fold lower than estimated by the recently developed Meta-CS single-cell duplex sequencing method³⁵, with a distinct pattern (Supplementary Table 2). Altogether, by direct interrogation of unamplified single molecules, HiDEF-seq achieves the highest fidelity for single base substitutions of any DNA sequencing method to date.

### Single-Strand Mismatch Patterns in Cancer Predisposition Syndromes

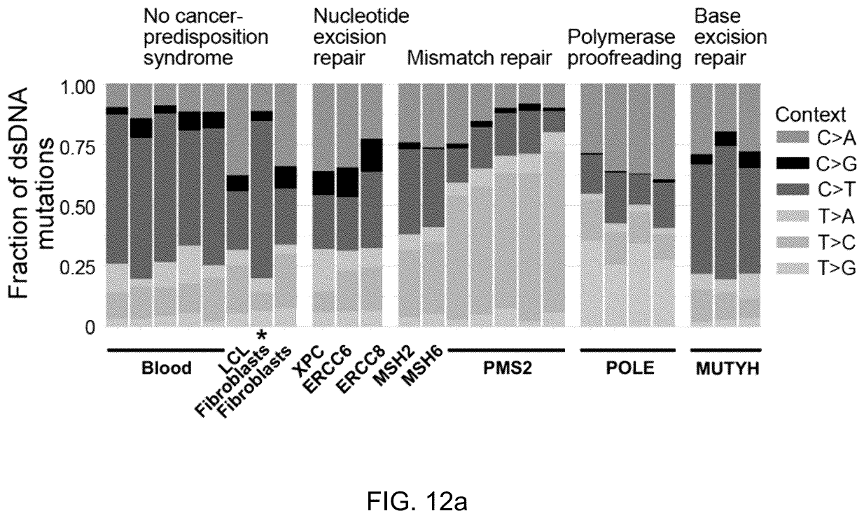

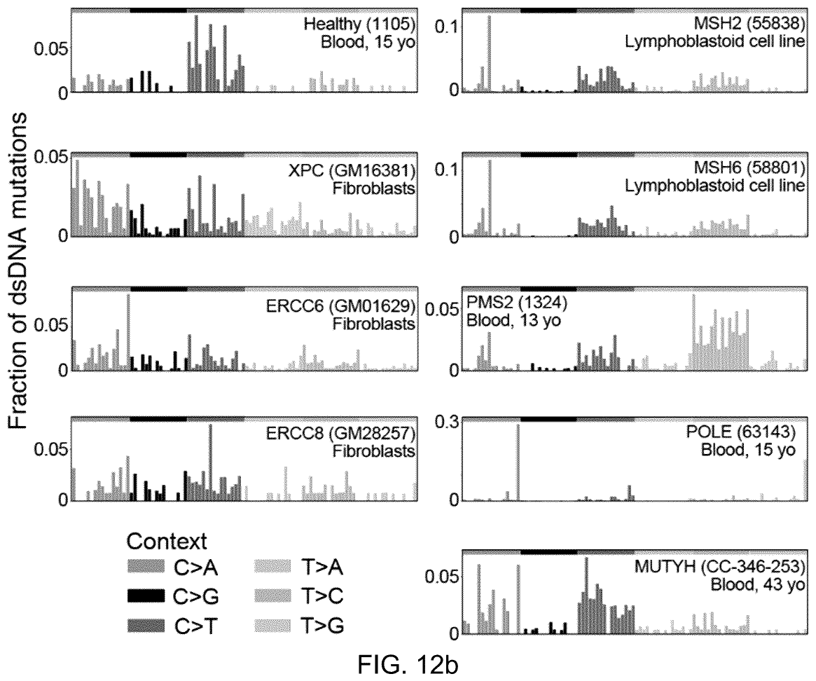

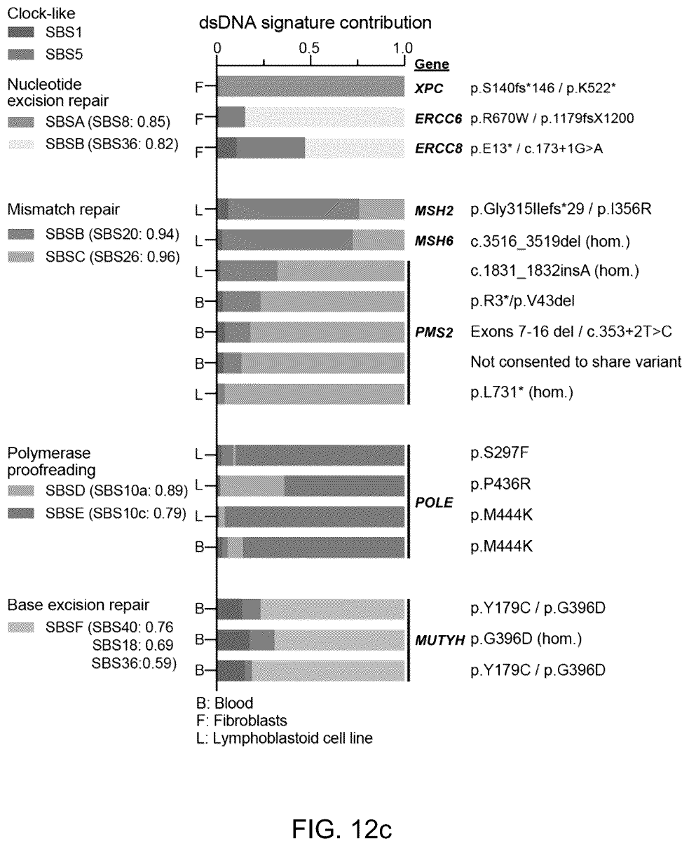

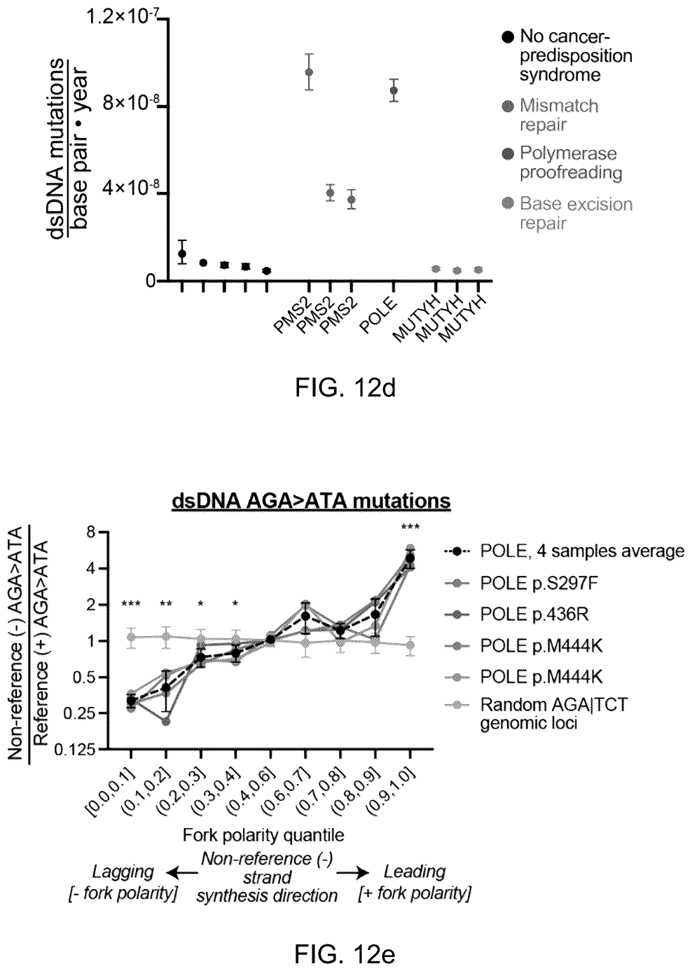

Since there is no prior method for sequencing ssDNA mismatches with single-molecule fidelity, we sought to confirm the veracity of HiDEF-seq's ssDNA calls by profiling samples and cell lines from individuals with inherited cancer predisposition syndromes that may have elevated ssDNA mismatch burdens. We profiled with HiDEF-seq of 17 blood, primary fibroblasts, and lymphoblastoid cell lines from 8 different cancer predisposition syndromes, including defects in nucleotide excision repair, mismatch repair, polymerase proofreading, and base excision repair (Supplementary Tables 1-2). In these samples, we first confirmed HiDEF-seq's fidelity for dsDNA mutations by measuring the expected dsDNA mutation burdens and signatures based on prior studies^{24,36-39}, except for MUTYH blood samples from which we were unable to recover its known signatures, since as seen in prior studies, MUTYH blood has near normal mutation burdens²⁴ (FIGS. 12a-d, and Supplementary Table 2). In ERCC6 and ERCC8 mutant cell lines, whose mutational patterns are unknown, we identified a signature similar to the COSMIC³³ SBS36 signature (SBS, single base substitution; cosine similarity 0.82) (Data FIGS. 12b-c). These data further illustrate the single-molecule fidelity of HiDEF-seq for dsDNA mutations.

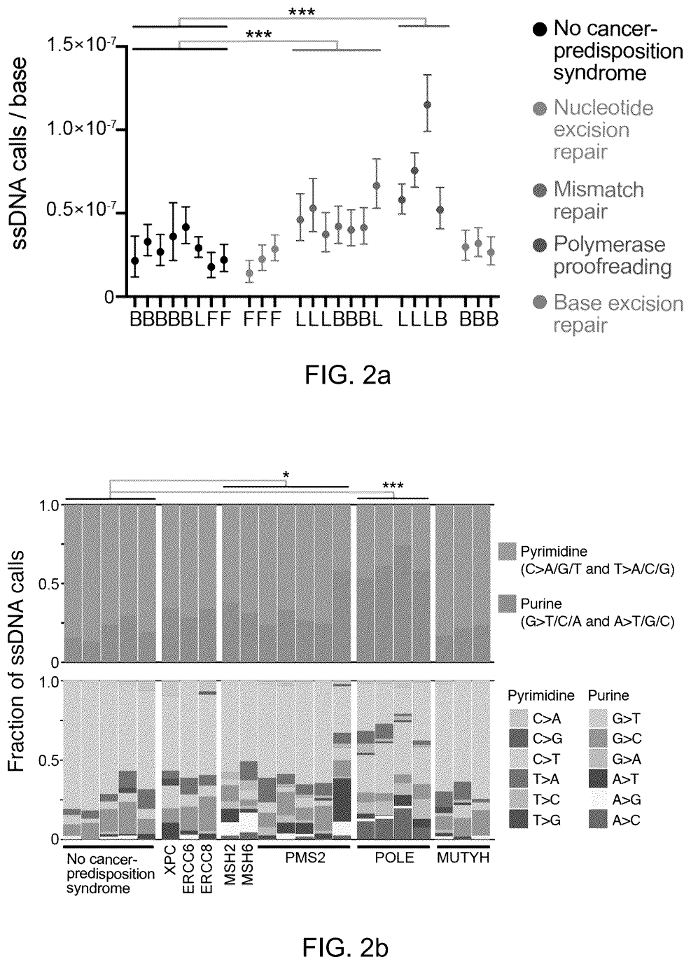

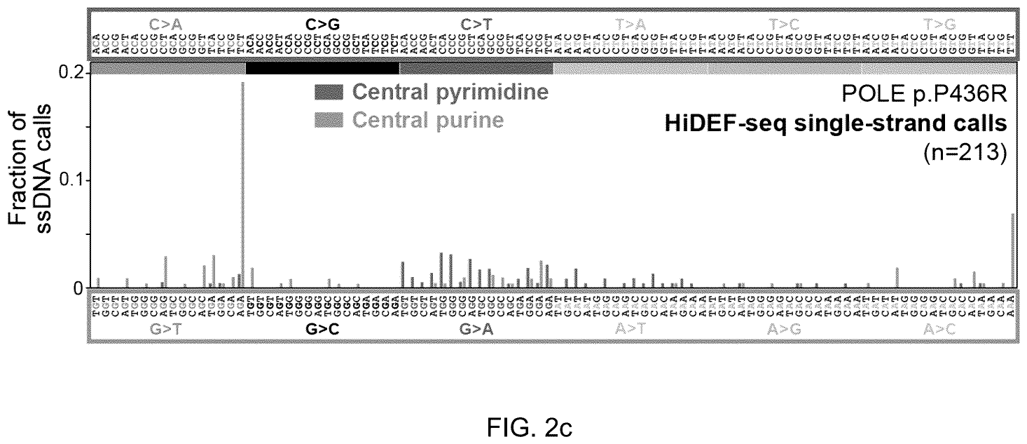

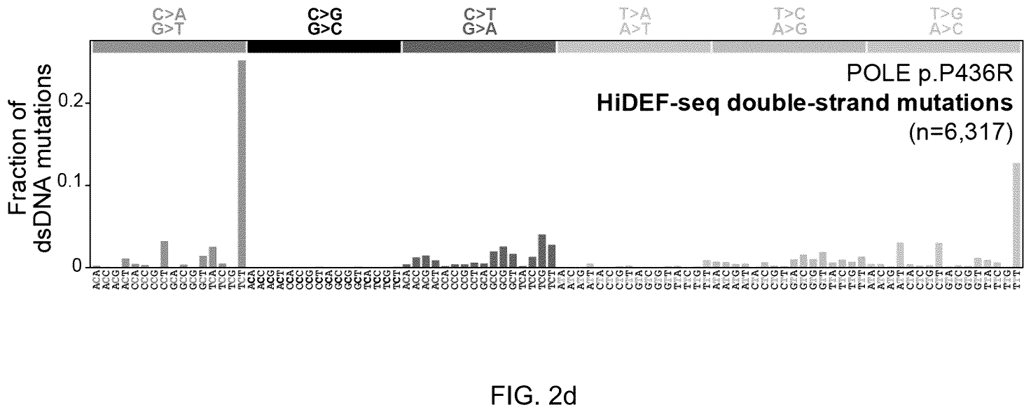

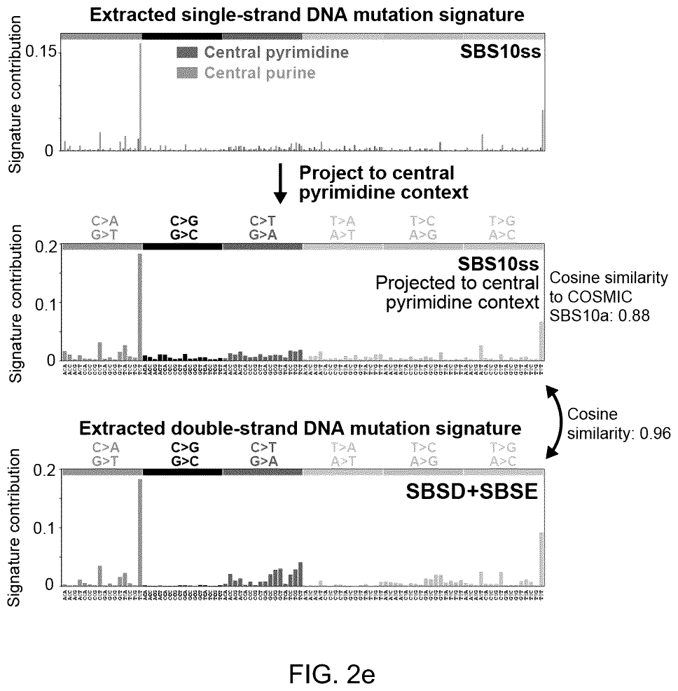

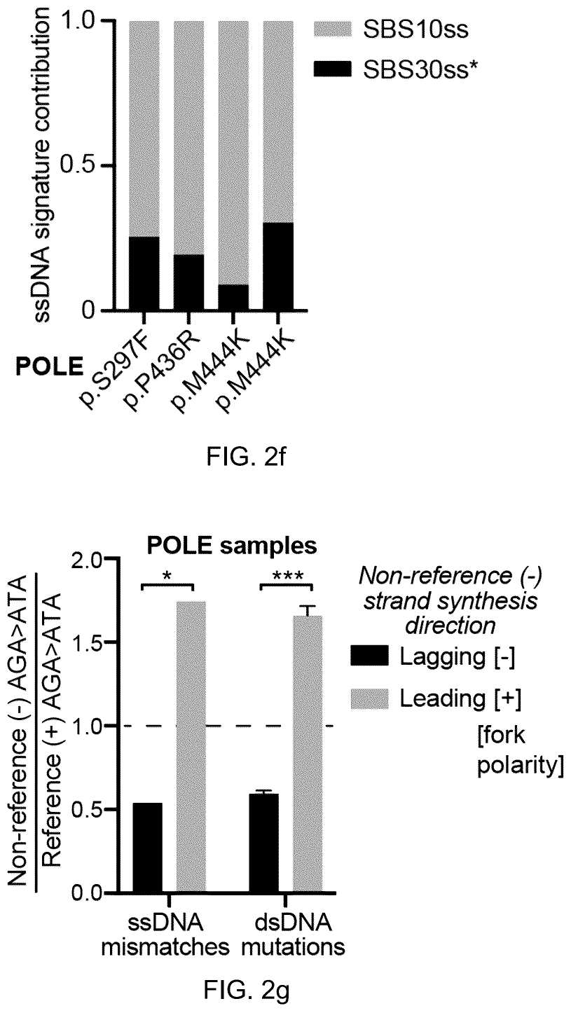

Notably, compared to non-cancer predisposition samples, we detected an increase in ssDNA calls per base in two cancer predisposition syndromes: a 2.6-fold increase (95% confidence interval 2.3-3.0, p<10⁻¹⁵, Poisson rates ratio test) in polymerase proofreading-associated polyposis syndrome samples (PPAP; heterozygous exonuclease domain mutations in POLE, which encodes polymerase epsilon that is responsible for leading strand genome replication⁴⁰)⁴¹, and a 1.6-fold increase (95% confidence interval 1.4-1.9, p=8·10⁻¹¹) in congenital mismatch repair deficiency syndrome samples (CMMRD; MSH2, MSH6, and PMS2 bi-allelic loss-of function) (FIG. 2a).

Next, we examined the patterns of ssDNA calls. The percentage of purine ssDNA calls (G>T/C/A and A>T/G/C) was elevated in PPAP samples to an average of 61% (range 53-74%) compared to 20% (range 13-29%) in non-cancer-predisposition samples (FIG. 2b; p=0.0004, heteroscedastic two-tailed t-test; analysis excludes non-cancer predisposition samples with less than 30 ssDNA calls as their call patterns are not reliably ascertained). This increase in purine ssDNA calls in PPAP was largely due to an increase in the fraction of G>T, and G>A, and A>C ssDNA calls (FIG. 2b). There was no significant correlation of ssDNA call contexts with specific germline POLE mutations in PPAP samples (FIG. 2b). The percentage of purine ssDNA calls was also elevated to a lesser degree in CMMRD samples to an average of 33% (range 23-58%, p=0.04), though without a clear enrichment of a specific sequence context except for one PMS2 loss-of-function sample with increased A>T ssDNA calls (FIG. 2b). These data indicate that most ssDNA calls in PPAP samples, and at least some calls in CMMRD samples, are bona fide ssDNA mismatches.

To further characterize the patterns of ssDNA mismatches in POLE PPAP samples, we plotted their 192-trinucleotide context spectra (standard 96-trinucleotide context spectrum, separated by central pyrimidine versus central purine). This revealed a distinct pattern, with two large peaks for AGA>ATA and AAA>ACA accounting for ˜15-20% and ˜5-10% of ssDNA mismatches, respectively, in addition to smaller peaks with G>T, G>A, A>C, and C>T context (FIG. 2c). The ssDNA mismatch spectra were highly concordant with these same samples' dsDNA mutation spectra (FIG. 2d), confirming these are true ssDNA mismatches and that these initial mismatch events-due to polymerase epsilon nucleotide misincorporation-lead to the subsequent pattern of accumulated dsDNA mutations. We then performed de novo extraction of ssDNA mismatch signatures from POLE PPAP samples, which produced a signature we name SBS10ss (ss, single-strand) (FIG. 2e). Note, as this is the first ssDNA signature the disclosure includes a nomenclature with suffix ‘ss’ to distinguish ssDNA from dsDNA signatures. Projecting SBS10ss to central pyrimidine context, by summing central purine and central pyrimidine spectra, produced a spectrum remarkably similar (cosine similarity 0.96) to the dsDNA signatures extracted de novo (SBSD+SBSE) from these same samples (FIG. 2e), again indicating that the ssDNA mismatches are the inciting events subsequently leading to dsDNA mutations. SBS10ss also had high similarity to COSMIC SBS10a (cosine similarity 0.88) that has been previously associated with POLE PPAP³⁶. SBS10ss accounted for an average of 79% (range 75-91%) of ssDNA calls in POLE PPAP samples, with the remaining attributed to SBS30ss*, a ssDNA cytosine deamination damage signature described in the next section (FIG. 2f). For CMMRD samples, the number of ssDNA calls was too low to extract a mutational signature.

The two most frequent ssDNA mismatch contexts in POLE PPAP samples are also notable for the asymmetry of their prevalence relative to their reverse complements: AGA>ATA versus TCT>TAT (73 vs. 10 mismatches across all POLE samples; chi-squared p<0.0001) and AAA>ACA versus TTT>TGT (26 vs. 2 mismatches; chi-squared p<0.0001). These data provide the first direct observation that the dsDNA mutational context of AGA>ATA|TCT>TAT that is prevalent in POLE PPAP arises significantly more frequently from C:dT (template base:polymerase incorporated base) misincorporations rather than G:dA misincorporations, and that the dsDNA mutational context of AAA>ACA|TTT>TGT arises more frequently from T:dC than A:dG misincorporations. These results are consistent with prior studies that indirectly inferred this asymmetry using yeast⁴² and human tumors⁴³⁻⁴⁵ harboring polymerase epsilon exonuclease domain mutations, by identifying asymmetries in the prevalence of dsDNA mutation contexts relative to their reverse complement contexts depending on whether the mutation locus is preferentially replicated via leading versus lagging strand synthesis. In contrast to these studies that rely on replication timing data that imperfectly estimates the probability of leading versus lagging strand replication in a bulk sample to measure this asymmetry, our single-molecule detection of nucleotides that were misincorporated in vivo by replicative polymerases allows us to measure this asymmetry directly. We also applied the above studies' indirect replication timing approach and similarly found replication strand asymmetry for our POLE PPAP samples' AGA>ATA dsDNA mutations (FIG. 2g and FIG. 12e). We further show that the AGA>ATA ssDNA mismatches in these samples occur more frequently on the strand that is synthesized in the leading, rather than lagging direction, consistent with the role of polymerase epsilon in leading strand synthesis^{40,41} (FIG. 2g). Altogether, these results represent the first direct measurements of in vivo ssDNA mismatch burdens and patterns.

### Single-Strand Patterns of Cytosine Deamination Damage

We analyzed whether HiDEF-seq's ssDNA fidelity may also enable detection of rare ssDNA damage events at the single molecule level-specifically, base damage that leads to nucleotide misincorporation by the sequencer polymerase. Detecting these rare events would be useful for characterizing processes that damage DNA. A common form of DNA damage that leads to mutations is the deamination of cytosine (with or without preceding oxidation) to uracil, uracil glycol or 5-hydroxyuracil (uracil-species)⁴⁶⁻⁵¹. When these lesions are not repaired, they result in C>T transitions⁴⁶. We analyzed whether HiDEF-seq may detect these ssDNA cytosine to uracil-species events despite their low levels (estimated by mass spectrometry as low as ˜1 in 1.5 million bases in blood^{52,53}), since the damaged cytosines would be mis-sequenced as thymine.

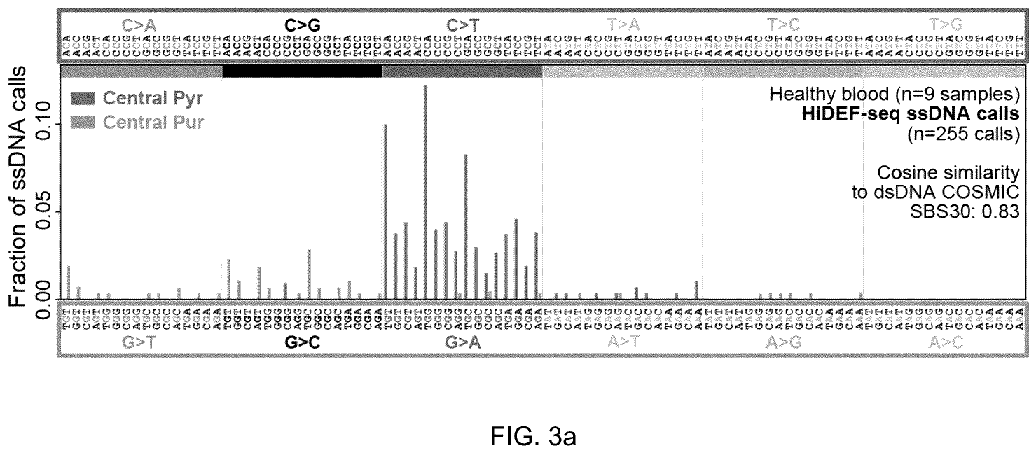

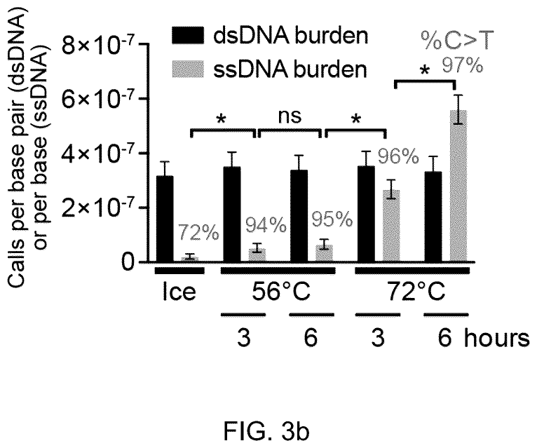

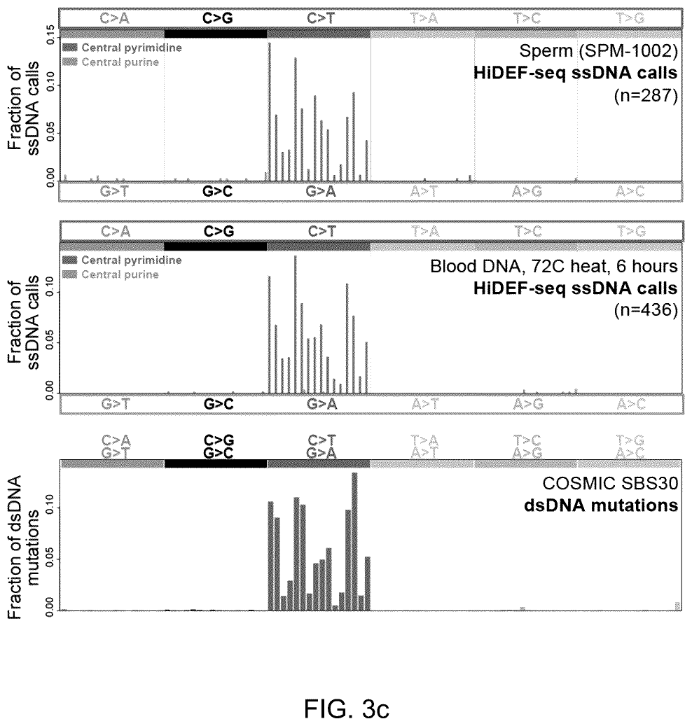

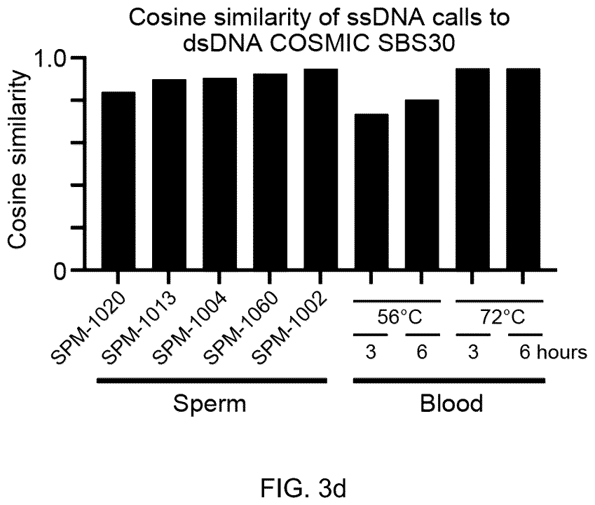

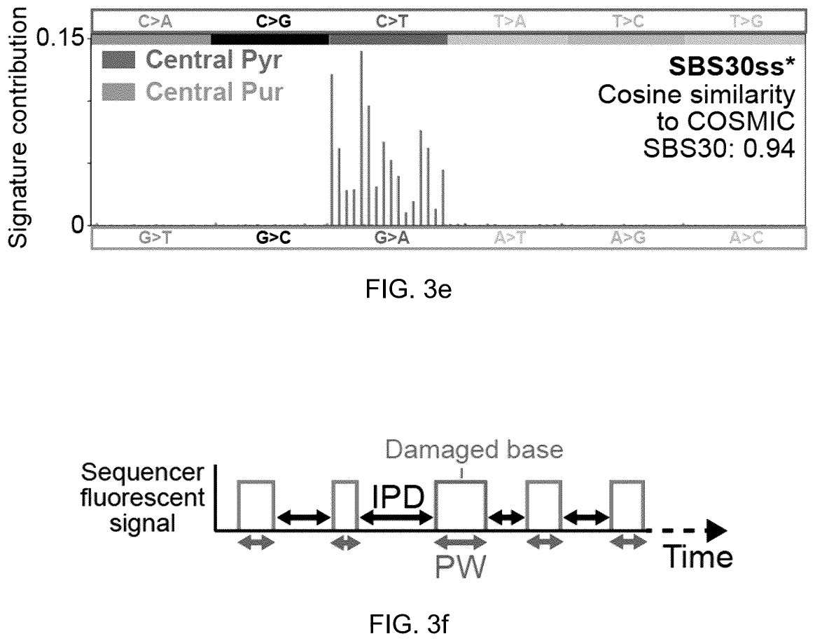

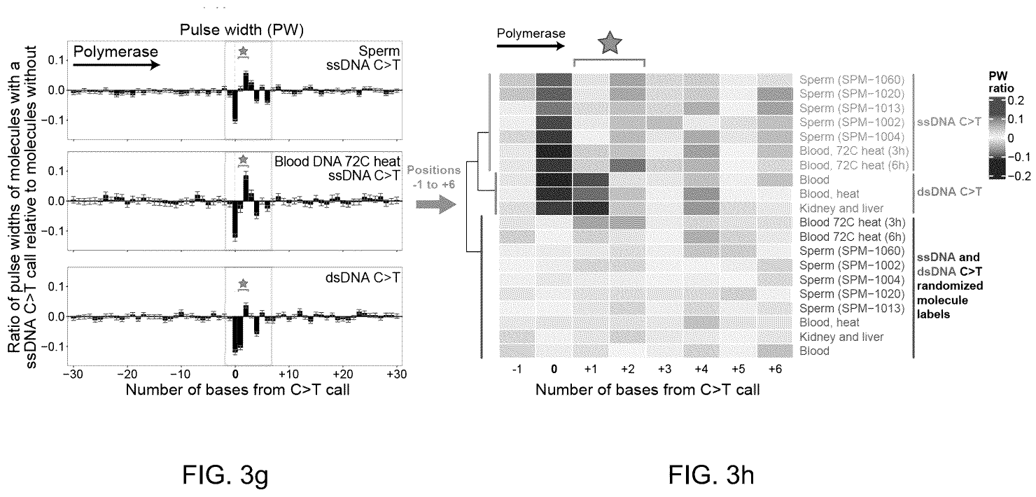

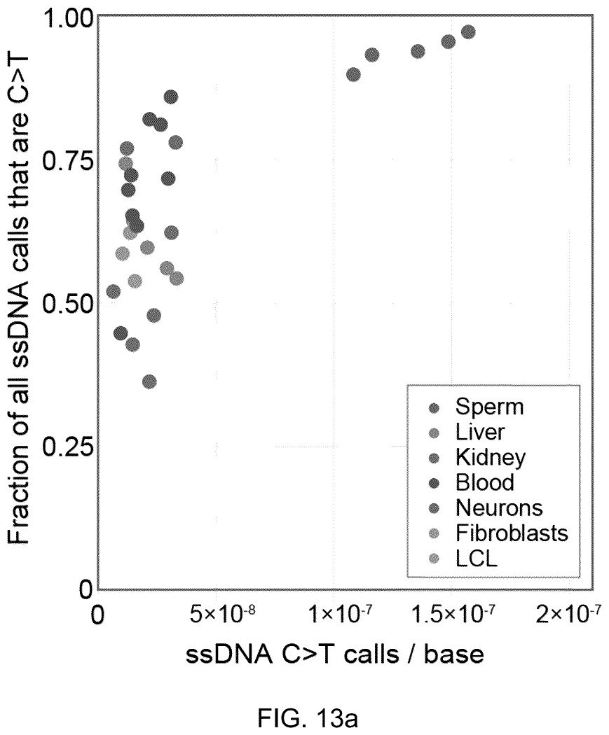

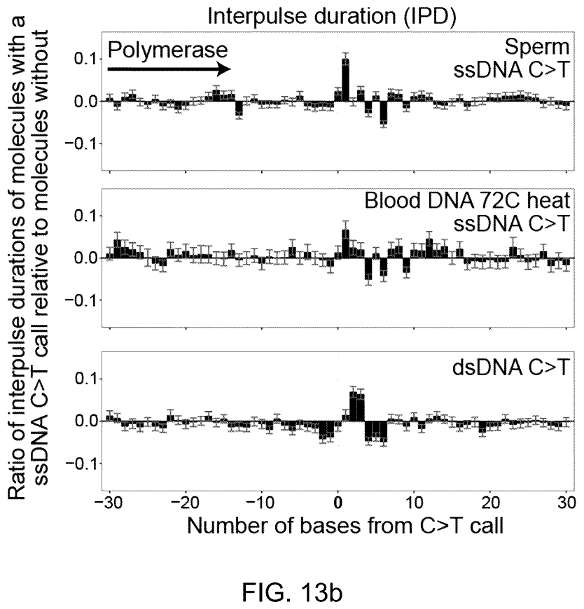

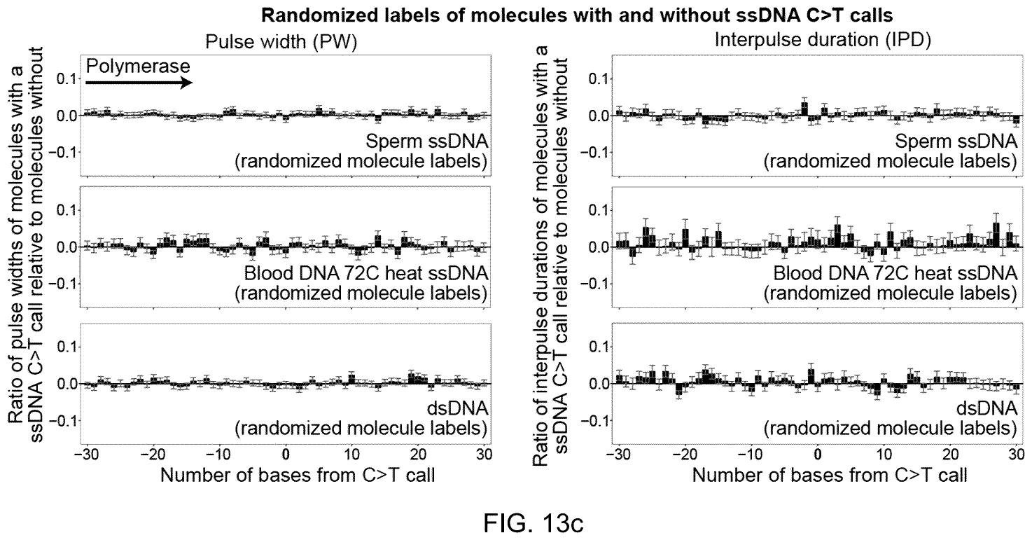

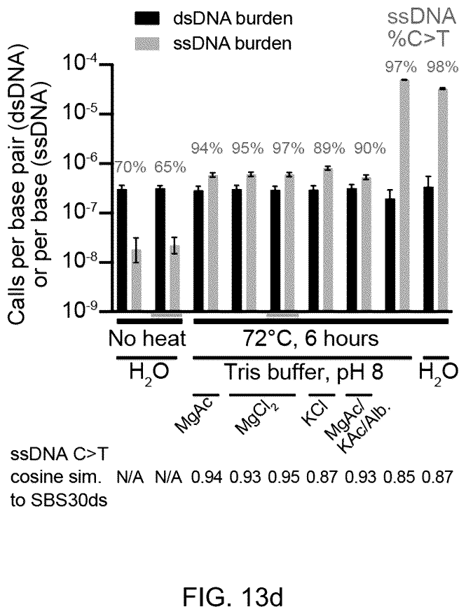

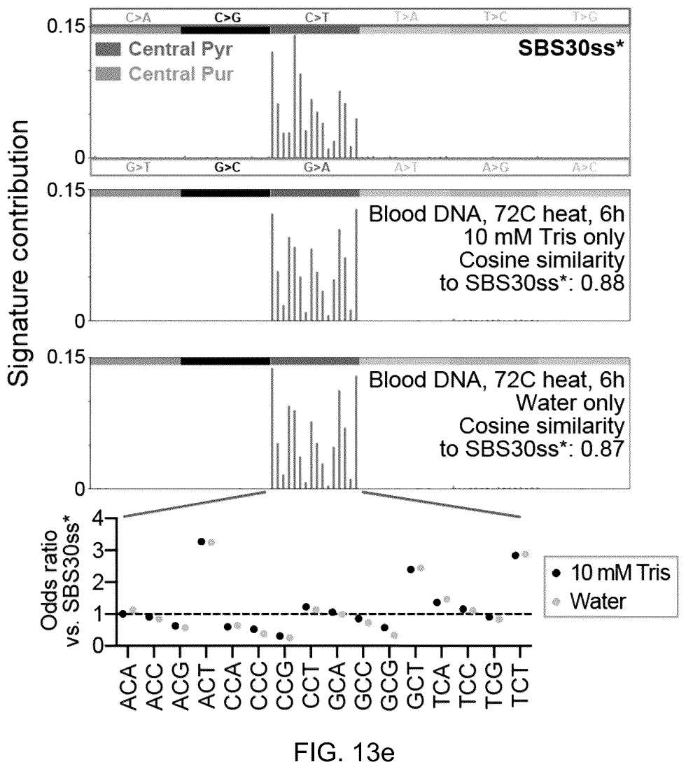

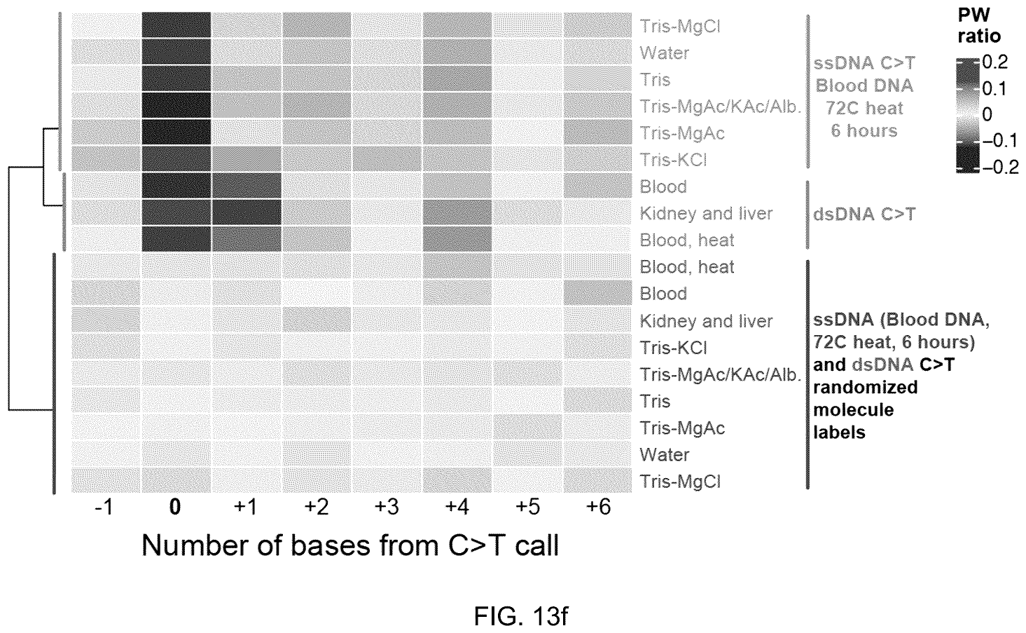

We began by investigating the burden and pattern of ssDNA C>T calls in blood DNA of non-cancer predisposition individuals, since blood is a primary tissue that can be stored and processed rapidly without potential post-mortem DNA damage. We also extracted the DNA with only room temperature incubations to avoid heat-induced oxidative deamination damage⁵⁴. Blood DNA had 2.1·10⁻⁸ ssDNA C>T calls per base (mean of n=9 samples from n=5 individuals; range 9.6·10⁻⁹-3.1·10⁻⁸), which comprised on average 72% of these samples' ssDNA calls (FIG. 13a and Supplementary Table 2). This burden, which may have either been present in vivo or may have partly arisen during laboratory processing of the blood or DNA, suggests there are fewer than 200 cytosine to uracil-species deaminated bases per cell in blood leukocytes-more than 30-fold lower than prior mass spectrometry studies of blood^{52,53}, which may have had higher background signal. This level of detection (1 event per 50 million bases) is on par with the most sensitive mass spectrometry methods^{55,56} and provides a low background for studying cytosine deamination processes. Notably, combining all the ssDNA calls across these samples and projecting their ssDNA trinucleotide spectrum to the corresponding dsDNA trinucleotide spectrum produced a spectrum similar to COSMIC³³ SBS30 (cosine similarity 0.83) (FIGS. 3a,c), a signature associated with cytosine oxidative deamination damage repaired by DNA glycosylases^{37,57-60}. Surprisingly, there was no signal in these blood samples for the commonly oxidized base 8-oxoguanine that would be expected to lead to G>T ssDNA calls, which were very infrequent in these blood samples (average 6% of ssDNA calls; 1.6·10⁻⁹ ssDNA calls per base; range 0-2.9·10⁻⁹). This is likely due to the sequencer polymerase (a derivative of phi29 polymerase) incorporating correctly mostly dC rather than misincorporating dA across from 8-oxoguanine bases⁶¹.

Given the high sensitivity of HiDEF-seq's ssDNA C>T detection, we sought to further elucidate the pattern of cytosine deamination damage in DNA via a larger number of events by investigating the effect of heat, an important source of laboratory-based cytosine deamination artifacts (since most DNA extraction methods utilize heat)^{54,62}. We profiled purified blood DNA after heat incubation at 56 C and 72 C, each for 3 and 6 hours. While heat did not affect the dsDNA mutation burden, HiDEF-seq measured a significant increase in ssDNA calls (29-fold for 72C, 6 hour treatment), specifically C>T calls, with increasing temperature and time (FIG. 3b and Supplementary Table 2). The effect of temperature was larger than the effect of time, and increased time had a larger effect at higher temperature (FIG. 3b). Additionally, after 56 C and 72 C heat treatment, 94% and 97% of ssDNA calls, respectively, were C>T. Observation of this effect of heat led us to profile almost all samples in this study except for one sample (neurons of individual 5344) at least once with a room temperature DNA extraction (i.e. without heat incubation) (Methods and Supplementary Table 1). Notably, the temperature during HiDEF-seq library preparation does not exceed 37 C (Methods).

Before analyzing the patterns of these ssDNA C>T calls, we surveyed all the healthy tissues and cell lines that we profiled by HiDEF-seq, and we found that compared to heat-treated samples, sperm had a similarly high percentage (average 94%) of ssDNA calls that were C>T as well as a high ssDNA C>T burden relative to other sample types (average 1.4·10⁻⁷ C>T calls per base) (FIG. 13a). This suggests these are also cytosine deamination events and that sperm DNA either undergoes a greater degree of in vivo cytosine deamination than DNA of other tissues, or that it incurs this damage ex vivo prior to sperm purification from semen, during sperm purification, and/or during DNA extraction. To distinguish among these possibilities, we processed blood DNA with a known low ssDNA C>T burden with the same process used to extract DNA from sperm. This did not produce an increase in ssDNA C>T burden, thereby excluding our method of sperm DNA extraction as the cause of sperm DNA's high burden of ssDNA C>T events (Supplementary Table 2). To assess the possible contribution of the sperm purification process to the ssDNA C>T burden⁶³, we purified sperm from semen of two additional individuals via filter chips that mimic physiologic separation of motile sperm, in parallel with the standard density gradient centrifugation method we used for the prior sperm samples (Methods). For each individual, sperm purified via the filter chip and sperm purified via density gradient centrifugation harbored the same percentage of ssDNA calls that were C>T (97% vs. 97% for the two methods for the first individual, and 87% vs. 87% for the second individual) and similar ssDNA C>T call burdens (1.4·10⁻⁷ vs. 1.1·10⁻⁷ for the first individual, and 9.0·10⁻⁸ vs. 9.0·10⁻⁸ for the second individual; p>0.05 for both comparisons) (Supplementary Table 2). These results suggest that the higher cytosine deamination burden in sperm occurs either in vivo or ex vivo during the time (<1 hour) that semen liquefies in the laboratory prior to sperm purification. In both cases, the elevated cytosine deamination burden in sperm would likely be present in sperm fertilizing the egg, which would then be repaired by the DNA repair machinery of eggs after fertilization^{64,65}.

Next, we analyzed the patterns (i.e., trinucleotide context spectra) of ssDNA C>T calls of sperm and heat-treated blood DNA samples. Strikingly, all sperm and heat-treated samples exhibited very similar ssDNA C>T spectra, and moreover, after projecting these ssDNA spectra to their corresponding dsDNA trinucleotide spectra, they again closely matched the COSMIC dsDNA signature SBS30 (average cosine similarity 0.90 and 0.95 for sperm and 72 C heat samples, respectively) (FIGS. 3c,d). Using all the above sperm and heat damage samples, we then extracted this ssDNA signature, which we term SBS30ss* (ss, single-strand; * indicates damage; cosine similarity 0.94 to SBS30) (FIG. 3e). The COSMIC dsDNA signature SBS30 has been previously associated with NTHL1 and UNG biallelic loss of function mutations^{37,57,58} and with formalin fixation⁶⁶. NTHL1 and UNG encode DNA glycosylases that initiate base excision repair of oxidized pyrimidines, including uracil-species that result from cytosine deamination^{59,60}. Our finding that in vitro heat treatment of purified DNA leads to a ssDNA damage signature, SBS30ss*, that matches the in vivo dsDNA SBS30 signature, indicates that SBS30ss* and SBS30 reflect the nucleotide context bias of the primary biochemical process of cytosine deamination, likely via an oxidized intermediate, rather than a bias of base excision repair glycosylases to more efficiently repair some trinucleotide contexts. Moreover, the correspondence of SBS30ss* to SBS30 indicates that in vitro heat damage and formalin fixation converge on the same in vivo biochemical process that is revealed by loss of NTHL1 and UNG.

To further confirm that ssDNA C>T calls in heat-treated DNA and sperm DNA are cytosine damage (i.e., uracil-species) rather than ssDNA changes of cytosine to thymine, we took advantage of the single molecule sequencer's real-time polymerase kinetic data that records at a 10 millisecond frame rate both the duration of each individual nucleotide incorporation (pulse width, PW) and the time between consecutive nucleotide incorporations (inter-pulse duration, IPD) (FIG. 3f). The patterns of PW and IPD as the sequencing polymerase replicates and traverses a damaged base across the polymerase's footprint of ˜7 nucleotides, encodes a unique kinetic signature for each canonical and damaged base^{67,68}. Kinetic signatures have been previously identified for diverse base modifications in synthetic oligonucleotides, and they have been used to detect a small number of base modifications in genomic DNA such as cytosine methylation^{67,69}. However, this approach has not yet been used to detect uracil-species in genomic DNA.

We began this kinetic analysis by extracting PW and IPD measurements from all ssDNA C>T calls of sperm and heat damage samples. We then controlled for local sequence context by normalizing the kinetic data of each molecule that has a C>T call using the kinetic data of all other molecules (across all samples) without C>T calls that aligned to the same locus (Methods). In parallel, we performed the same analysis for dsDNA C>T mutations (for the strand containing the thymine), as these are bona fide cytosine to thymine mutations rather than cytosine damage. This analysis revealed a distinct kinetic signature for ssDNA C>T calls that differed from that of dsDNA C>T mutations (FIG. 3g and FIG. 13b). Unbiased hierarchical clustering of PW of the −1 to +6 positions, which corresponds to the polymerase footprint in which the kinetic signal differs from the flanking baseline, separated the kinetic profile of individual samples' ssDNA C>T calls from dsDNA C>T mutations (FIG. 3h and FIG. 13f). Randomizing molecule labels produced a flat baseline, further confirming the validity of the kinetic signatures (FIG. 3h and FIG. 13c). These results provide further evidence that the ssDNA C>T calls are uracil-species arising from cytosine deamination and definitively exclude the possibility that they are cytosine to thymine mutations.

We further tested whether the DNA extraction method used prior to heat treatment affected cytosine deamination, as well as heat treatment of DNA in 5 different Tris-buffered solutions and in water. DNA extraction with and without iron-containing magnetic beads—to exclude the possibility that the heat deamination is induced by oxidation due to iron leached from the beads—and all salt-containing buffers produced a similar burden of cytosine to uracil-species damage and the same SBS30ss* pattern as described above; however, samples that were heat-treated in water or in Tris-buffer without additional salt had a further 65-fold increase in cytosine damage (FIG. 13d and Supplementary Table 2). Moreover, in these no salt and low salt samples, the damage pattern still closely matched SBS30ss*, except for an increased burden at DCT>DTT trinucleotide contexts and a decrease in burden for 5′ C and 3′ G contexts (FIG. 13e). Since low or absent salt decreases DNA duplex stability at elevated temperatures and makes DNA more susceptible to oxidative damage^{70,71}, these results suggest that the in vivo mechanism of the SBS30ss* signature, and consequently the dsDNA SBS30 signature, is cytosine deamination of DNA while it is transiently single-stranded.

### Single-Strand DNA Calls in Healthy Tissues

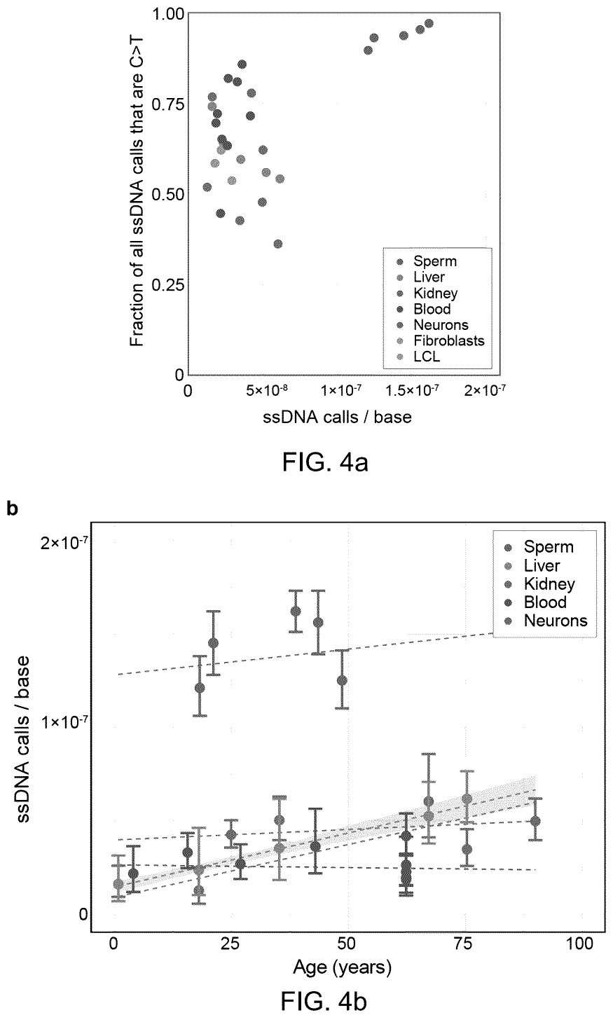

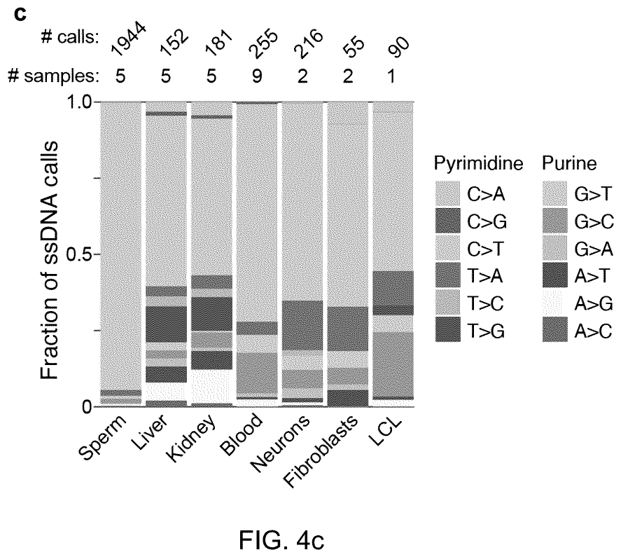

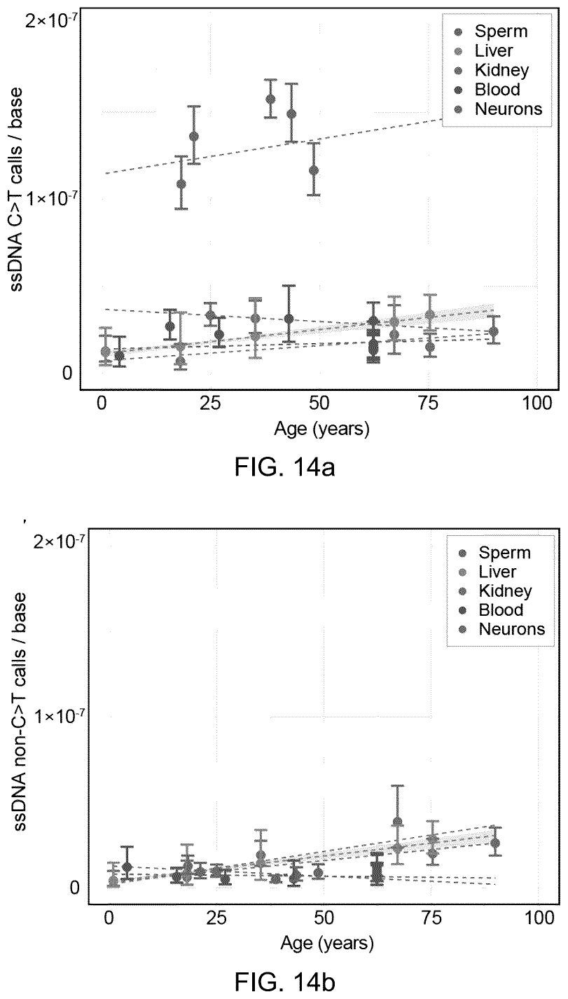

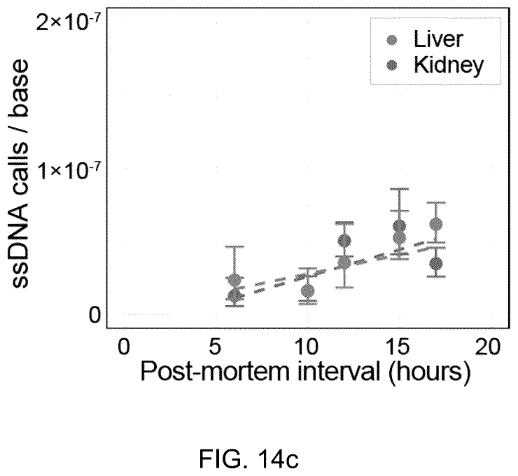

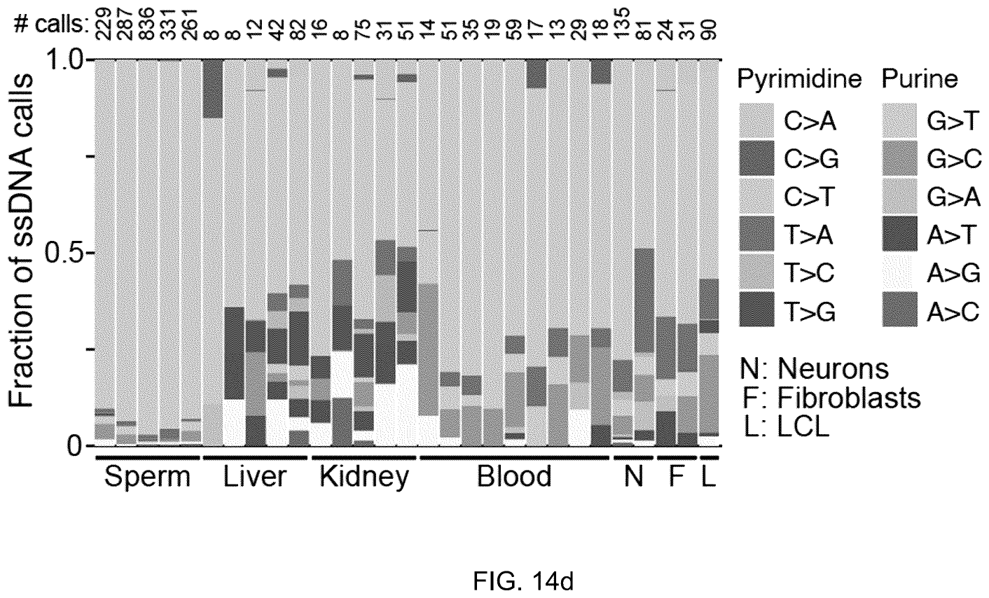

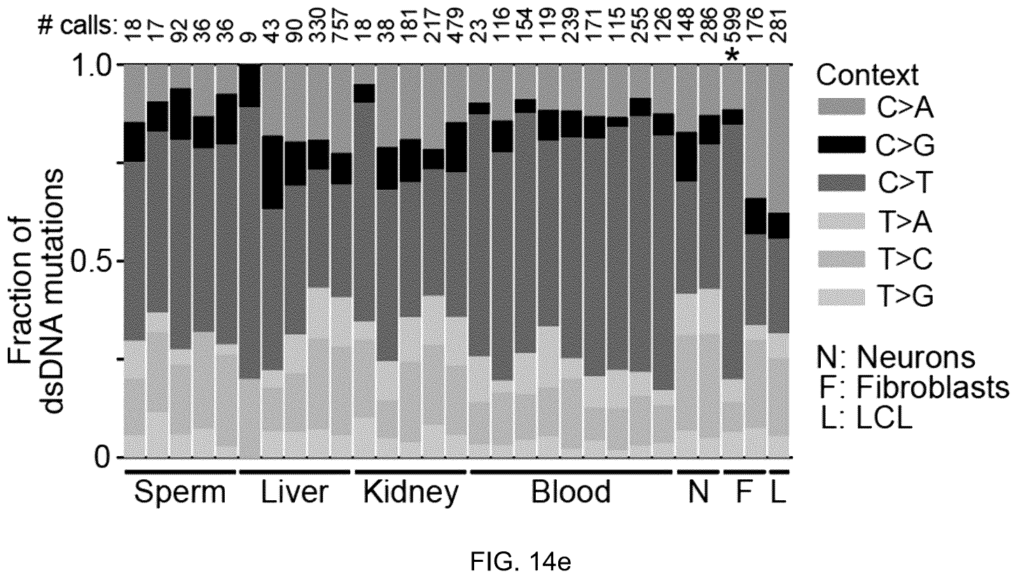

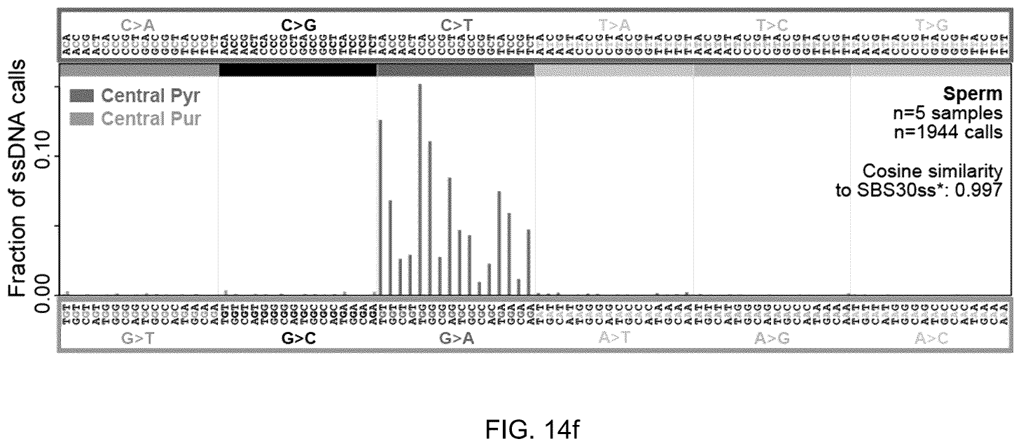

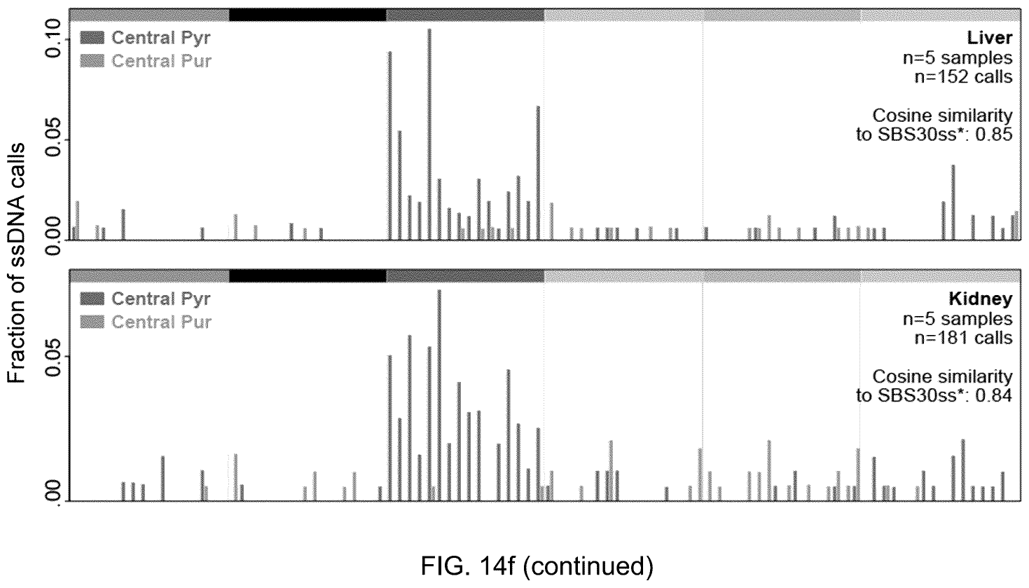

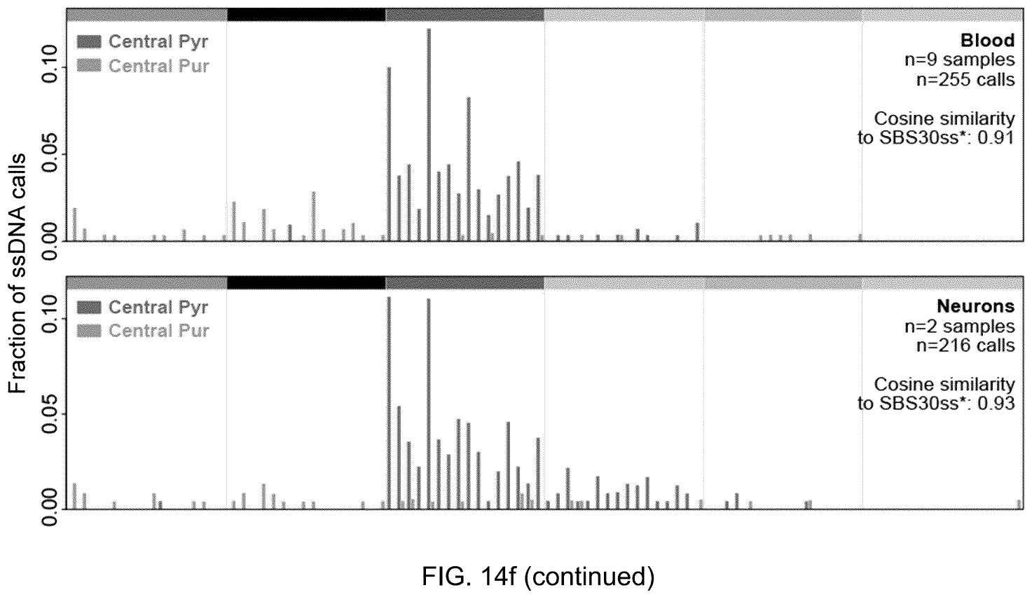

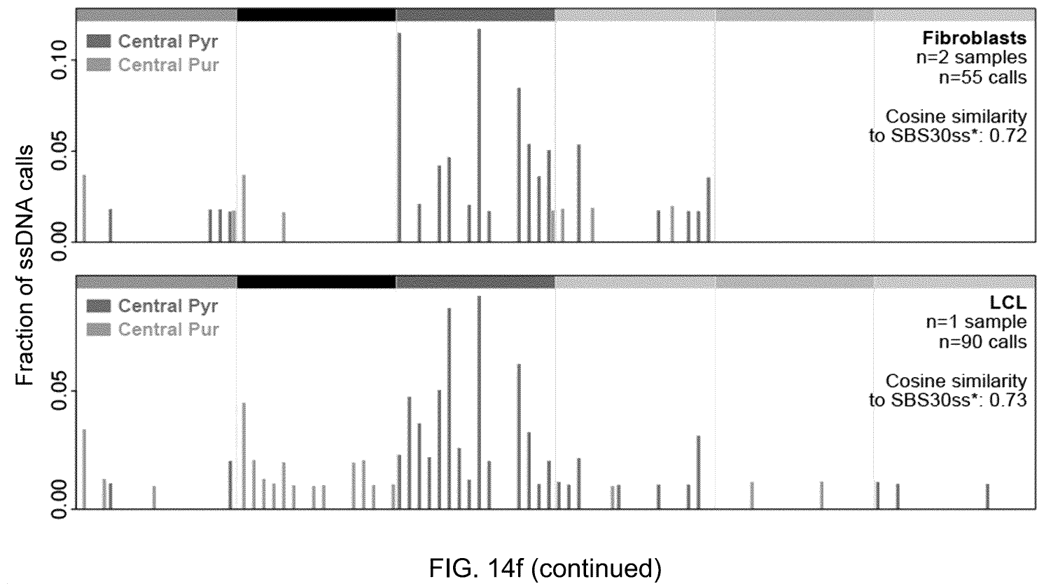

We examined the burdens and patterns of ssDNA calls across the 29 healthy (i.e., non-cancer predisposition) samples that we profiled from sperm, liver, kidney, blood, cerebral cortex neurons, primary fibroblasts, and a lymphoblastoid cell line (n=2,893 calls; 83% C>T). Except for sperm that exhibit significantly elevated ssDNA C>T calls from cytosine deamination damage as described above, we did not observe significant differences in ssDNA call burden among tissue types (FIG. 4a). Liver samples, but not other tissues, showed a small but statistically significant increase in ssDNA call burden with age (5.8·10⁻¹⁰ calls per year; p=0.0005) (FIG. 4b), and this correlation with age decreased but remained statistically significant after including post-mortem interval (PMI) as a covariate in a multiple linear regression model (5.4·10⁻¹⁰ calls per year; p=0.002). This finding for liver tissue persisted when analyzing only ssDNA C>T calls (2.5·10⁻¹⁰ calls per year; p=0.002) and ssDNA non-C>T calls (2.9·10⁻¹⁰ calls per year; p=0.002) when including PMI as a covariate (FIGS. 14a,b). However, ssDNA call burdens tended to increase with PMI in post-mortem kidney and liver samples (FIG. 14c), and although this association with PMI was not statistically significant, since other tissues did not exhibit an increase in ssDNA burden with age, it is possible that PMI does not fully capture post-mortem effects that may explain the increase in ssDNA calls with age in liver.

Analysis of single nucleotide sequence contexts of ssDNA calls (i.e., C>A, C>G, C>T, etc.) across tissues was notable for the high fraction of C>T calls in sperm and a small increase in the fraction of T>G and A>G calls in post-mortem kidney and liver (FIG. 4c and FIGS. 14d,e). The latter A>G calls may be due to 8-oxoadenine DNA damage occurring either pre or post-mortem⁷². ssDNA call spectra of all tissues were similar to SBS30ss* (cosine similarities 0.72-0.93 in non-sperm tissues and 0.997 in sperm) (FIG. 14f), which may be either due to endogenous or ex vivo cytosine deamination. No other ssDNA signature was identified, likely due to the low ssDNA call burdens in healthy tissue samples. Further studies of ssDNA mismatch patterns in healthy tissues, and whether these increase with age in some tissues, will require higher throughput single-molecule sequencing instruments.

### Single- and Double Strand DNA Event Burdens in the Mitochondrial Genome

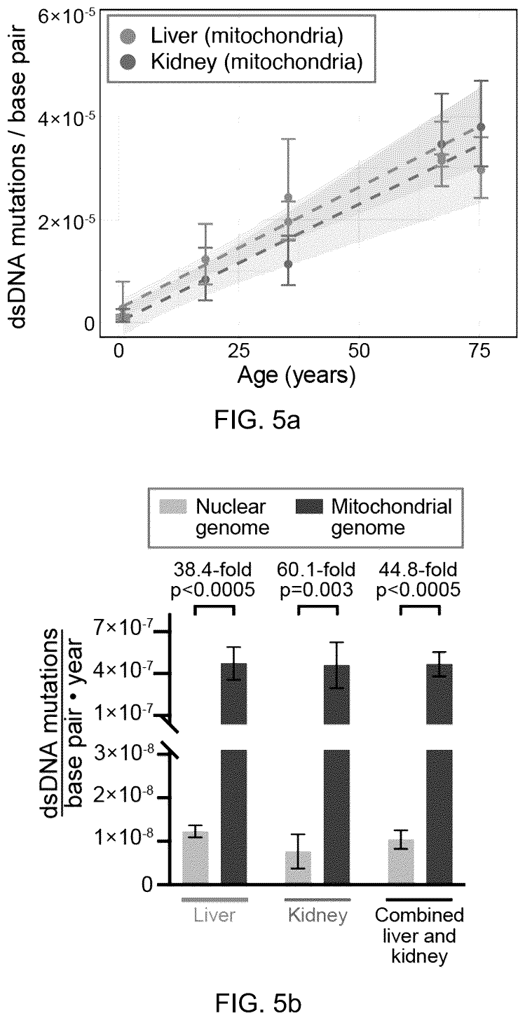

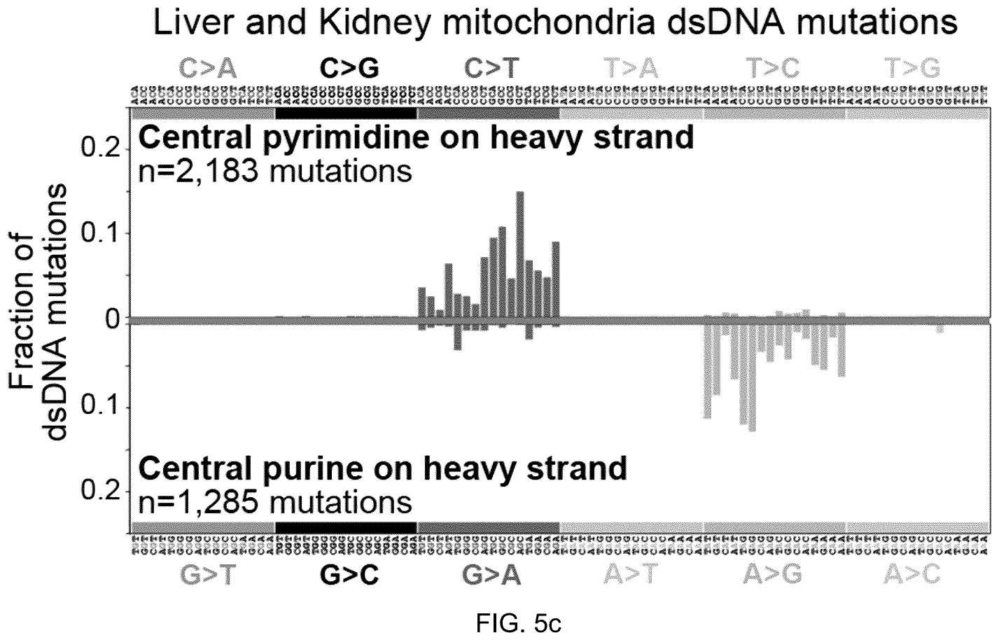

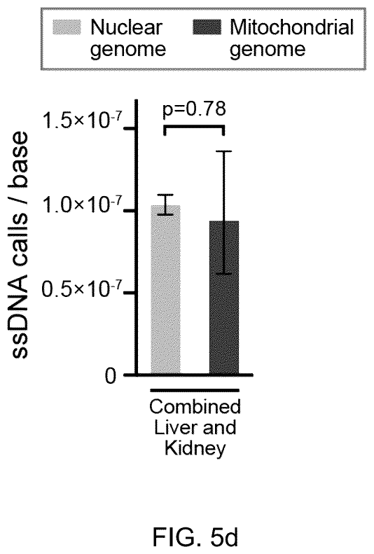

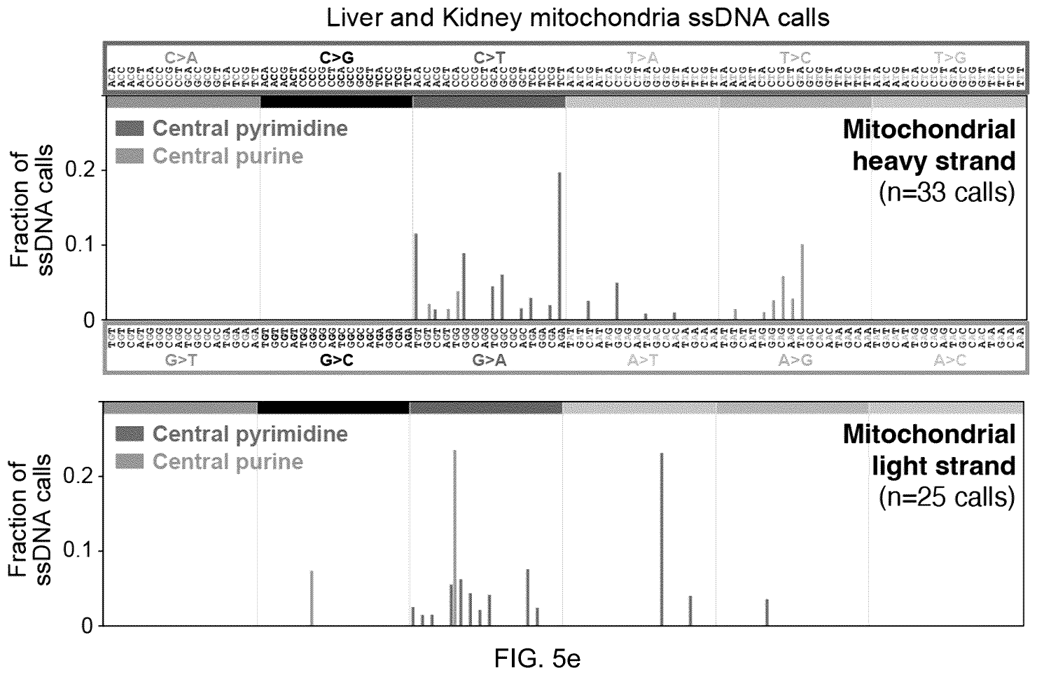

Prior studies have measured an ˜20-40-fold higher somatic dsDNA mutation rate with age in the mitochondrial genome than the nuclear genome¹⁵. However, the mechanism by which the mitochondrial genome mutates remains unclear⁷³⁻⁷⁸. While it was long assumed to be due to oxidative damage from mitochondrial oxidative metabolism^{75,79}, recent studies have not identified oxidation-related mutational signatures such as G>T mutations from 8-oxoguanine, and instead have found patterns supporting a mechanism closely linked to replication^{73-75,77,80,81}. Specifically, A>G and C>T dsDNA mutations are highly enriched on the mitochondrial heavy strand (the G+T-rich reference genome ‘-’ strand, that is the template strand for most genes)—i.e., A and C nucleotides on the heavy strand mutate to G and T, respectively, and complementary changes on the opposite strand—with a gradient in frequency that decreases with distance from the origin in the direction of replication^{73,74,77,80}. Several potentially overlapping hypotheses have been proposed for these findings: a) the mitochondrial genome's strand-displacement mechanism of replication leaves the heavy strand exposed for a longer time as single-stranded DNA, making it vulnerable to deamination of adenine and cytosine that are then mispaired with cytosine and adenine, respectively, during replication^{73,74,77,82-84}; b) strand asymmetries in polymerase misincorporation of canonical nucleotides^{74,75,78}; and, c) strand asymmetries in DNA repair⁷⁴. Importantly, if DNA repair is not substantially more efficient in mitochondria than the nuclear genome⁸⁵, we would expect HiDEF-seq to detect the latter two possibilities as ssDNA mismatches of canonical nucleotides, since HiDEF-seq detects an increased burden of ssDNA mismatches of canonical nucleotides in the nuclear genomes of mismatch repair-deficient and POLE PPAP samples that have even lower dsDNA mutation rates than mitochondria: 8.1 and 5.4-fold lower, respectively (FIGS. 5a,b and FIG. 12d). Since HiDEF-seq captures molecules from one third of the mitochondrial genome (Methods), we investigated mitochondrial dsDNA and ssDNA call burdens and patterns to distinguish among these hypotheses.

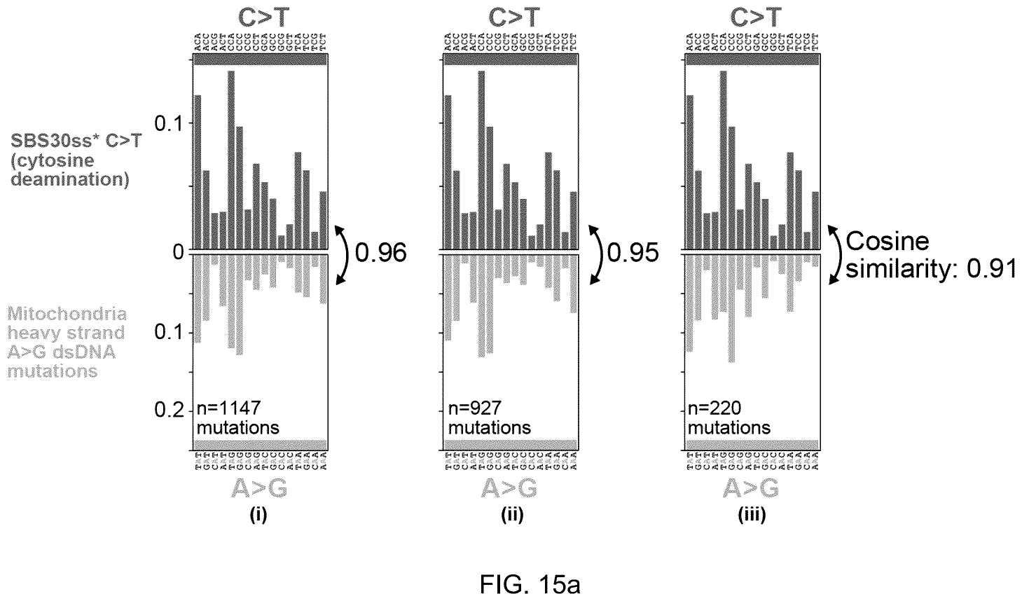

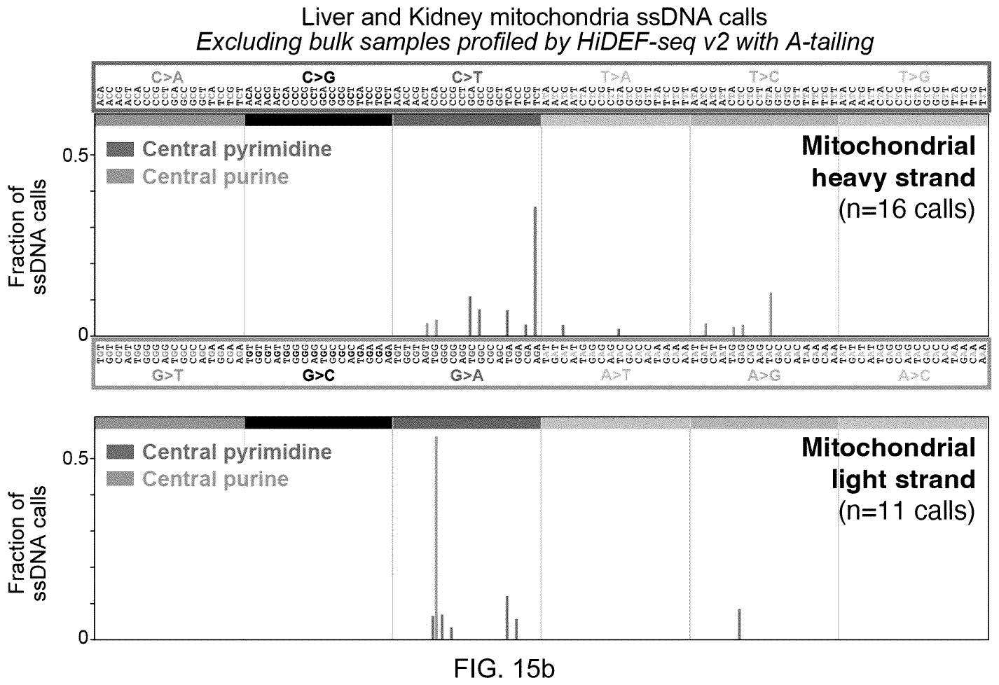

We focused on liver and kidney samples, which had a higher yield of mitochondrial DNA (average 1% of sequenced molecules per sample) than other tissues (Supplementary Table 1). Additionally, we purified mitochondria from 3 liver samples, which further increased the yield of mitochondrial DNA (average 13% of sequenced molecules per sample; Supplementary Table 1). We detected the expected increase in mitochondrial dsDNA mutation burden with age (FIG. 5a), and this rate was 38.4- and 60.1-fold higher in liver and kidney, respectively, than the dsDNA mutation rate of these tissues' nuclear DNA (FIG. 5b). Combining liver and kidney samples, the difference was 44.8-fold (FIG. 5b). HiDEF-seq also detected the expected highly asymmetric pattern of A>G and C>T dsDNA mutations on the heavy strand, though with different distributions of peaks than prior cancer studies^{74,80} (FIG. 5c). There was no significant similarity of the full mitochondrial mutation spectrum to COSMIC signatures, and the spectrum did not exhibit the NCG>NTG or NTC>NCC peaks seen in prior bulk cancer sequencing studies^{74,80}. However, there was significant similarity between the A>G portion of the heavy strand spectrum and the C>T SBS30ss* cytosine deamination and COSMIC SBS30 signatures (cosine similarities 0.96 and 0.92, respectively) (FIG. 15a). The mechanism for this similarity is unclear, but this finding suggests a deamination mechanism for A>G heavy strand mitochondrial mutagenesis (37% of dsDNA mutations in our data) and that the same biophysical effects that determine the propensity of cytosine to deaminate preferentially within certain trinucleotide contexts similarly affects adenine deamination.

Notably, despite the large differences in dsDNA mutation rates in the mitochondrial and nuclear genomes, their ssDNA call burdens were not significantly different (p=0.78, ANOVA) (FIG. 5d). Specifically, there were only 27 ssDNA calls in 2.7·10⁵ mitochondrial genome molecules interrogating 3.8·10⁸ bases of mitochondrial ssDNA. While the number of ssDNA calls was low, these were concentrated in sequence contexts consistent with the dsDNA mutation spectrum (FIG. 15b). To further assess if these patterns are consistent with specific mutagenic mechanisms, we increased the number of analyzed calls (n=58) by including liver and kidney samples previously profiled by HiDEF-seq v2 with A-tailing, since the ssDNA T>A artifact that A-tailing can incur is orthogonal to the contexts of mitochondrial mutagenesis. The spectrum of this larger call set was also consistent both with the dsDNA mutation spectrum and the following mechanisms of mutagenesis: cytosine deamination on the heavy strand (15/20 heavy strand central pyrimidine calls are C>T), adenine deamination on the heavy strand (8/13 heavy strand central purine calls are A>G), cytosine deamination on the light strand (18/22 light strand central pyrimidine calls are C>T), and polymerase G>A misincorporation on both strands (7/16 central purine calls are G>A), respectively (FIG. 5e).

Altogether, these data strengthen the evidence that the mitochondrial genome mutates during replication via deamination of cytosine and adenine on the heavy strand, while it is single-stranded, and to a lesser extent via deamination of cytosine on the light strand. Additionally, these results suggest that polymerase G>A misincorporation events may contribute to the dsDNA C>T mutation spectrum.

### Methods

### Sample Sources

Post-mortem human tissues were obtained from the NIH NeuroBioBank (University of Maryland site). Post-mortem tissues were frozen in isopentane-liquid nitrogen baths and stored at −80° C. until use. Human blood was obtained from individuals enrolled in human subjects research approved by the New York University Grossman School of Medicine Institutional Review Board, the International Replication Repair Deficiency Consortium (IRRDC) based at The Hospital for Sick Children (SickKids), and the University of Pittsburgh School of Medicine. All blood samples were collected in EDTA tubes and stored frozen until use. Semen samples were obtained at Cryos International Sperm Bank from individuals enrolled in human subjects research approved by the New York University Grossman School of Medicine Institutional Review Board. Lymphoblastoid cell lines were obtained from Coriell Institute and the International Replication Repair Deficiency Consortium. Primary fibroblasts were obtained from Coriell Institute.

Sources, sex, ages at collection, and post-mortem interval of each sample are listed in Supplementary Table 1.

### Cell Culture

Lymphoblastoid cells were cultured in T25 flasks with RPMI 1640 media (Thermo Fisher, product #61870036) supplemented with 15% fetal bovine serum and penicillin-streptomycin. Cells were incubated at 37 C, 5% CO₂, and ambient oxygen. Cells were passaged to new media approximately every 2-3 days.

Fibroblasts were cultured in T25 flasks with DMEM media (Thermo Fisher, product #10569010) supplemented with 10% fetal bovine serum and penicillin-strepomycin. Cells were incubated at 37 C, 5% CO₂, and ambient oxygen. Cells were passaged to new media every 3-5 days prior to reaching full confluency. Cells were harvested for DNA at 80-90% confluency using trypsin-EDTA.

### Sperm Purification

After collection, semen underwent liquefaction at room temperature for 30 to 60 minutes. Semen then immediately underwent initial purification for sperm using density gradient centrifugation followed by a wash with HEPES-buffered media⁸⁶. For semen from individuals D1 and D2, sperm was purified from half the semen sample by this method, and sperm was purified from the other half with a ZyMot Multi (850 μL) Sperm Separation Device (ZyMot) per the manufacturer's instructions. After addition of cryopreservation media, sperm were stored in liquid nitrogen until further use.

After thawing, sperm that previously underwent initial purification by density gradient centrifugation were further purified with a second density gradient centrifugation and two additional washes, as follows. First, the following reagents were equilibrated to room temperature—ORIGIO gradient 40/80 buffer (Cooper Surgical, 84022010), Origio sperm wash buffer (Cooper Surgical, 84050060), and Quinn's Advantage sperm freezing medium (Cooper Surgical, ART-8022). In a 15 mL tube, 1 mL of Origio 80 buffer was placed at the bottom of the tube. 1 mL of Origio 40 buffer was gently layered on top of the Origio 80 buffer. Sperm were thawed at room temperature for 15 minutes. Sperm were gently pipette mixed and carefully layered on top of the Origio 40 buffer and centrifuged in a swinging bucket centrifuge at 400×g for 20 minutes at room temperature with low acceleration and deceleration speeds. The supernatant was aspirated, leaving 500 μL of sperm/buffer at the bottom. The sperm was transferred to a new 15 mL tube and diluted with 5 mL sperm wash buffer. The tube was mixed by inverting 10 times and centrifuged in a swinging bucket centrifuge at 300×g for 10 minutes at room temperature with maximum acceleration and deceleration. The supernatant was removed, leaving about 350 μL of sperm/buffer at the bottom. The sperm was then washed again in the same way with 5 mL of sperm wash buffer, and the supernatant was removed to leave about 250 μL of sperm/buffer at the bottom of the tube. After pipette mixing, an aliquot of this sperm was then transferred to a 2 mL DNA LoBind microtube (Eppendorf) for immediate DNA extraction and microscopy quantification of somatic cell contamination using a hemoyctometer. The remaining sperm was diluted dropwise with a 1:1 volumetric ratio of sperm freezing medium, incubated at room temperature for 3 minutes, frozen in a Mr. Frosty freezing container (Thermo Fisher) at −80° C. freezer for 24 hours, and then transferred to a liquid nitrogen freezer.

### Cerebral Cortex Neuronal Nuclei Purification

Cerebral cortex neuronal nuclei were isolated from post-mortem tissue of individuals who did not have any known neurological or psychiatric disease: 1) Subject 5344 (Brodmann area 9, left hemisphere), and Subject 6371 (Brodmann area 9, left hemisphere). Approximately 1 gram of frozen tissue from each was cut into 5 mm³ pieces and added to 9 mL of chilled lysis buffer (0.32M sucrose, 10 mM Tris HCl pH 8, 5 mM CaCl₂, 3 mM magnesium acetate, 0.1 mM EDTA, 1 mM DTT, 0.1% Triton-X) in a large dounce homogenizer (Sigma-Aldrich D9938). While on ice, the tissue was dounced 20 times each with pestle size A and then B. The homogenate was layered on a 7.4 mL sucrose cushion (1.8M sucrose, 10 mM Tris HCl pH 8, 3 mM magnesium acetate, 1 mM DTT) in an ultra-centrifuge tube on ice. Tubes were centrifuged (Thermo Fisher Sorvall LYNX) at 10,000 rpm for 1 hour at 4° C. The resulting supernatant was removed, and 500 μL of nuclei resuspension buffer (3 mM MgCl₂ in 1× Phosphate-Buffered Saline) was added on top of the pellet and then incubated on ice for 10 minutes. The pellet was then gently resuspended. Antibody staining buffer was prepared by adding 1.2 μg of NeuN-Alexa-647 (abcam ab190565) to 400 μL of antibody staining buffer (3% BSA in nuclei resuspension buffer) and inverted gently to mix. 400 μL of antibody staining buffer was added to 1 mL of nuclei in a and rotated at 4° C. for 30 minutes. NeuN-positive nuclei were collected in 30 μL of nuclei buffer in 1.5 mL LoBind tubes via fluorescence-activated nuclei sorting (FANS) on an LE-SH800 sorter. After sorting, a 1:1 volumetric ratio of 80% glycerol was added to sorted nuclei for a final concentration of 40% glycerol to stabilize nuclei during centrifugation. Nuclei were centrifuged at 4° C., 500×g for 10 mins. Supernatant was removed and nuclei pellets were immediately frozen at −80° C.

### Extraction and Isolation of Mitochondria for HiDEF-Seq

Mitochondria were extracted and isolated from between 300-500 mg of tissue using the Mitochondria Extraction Kit (Miltenyi Biotec) and Mitochondria Isolation Kit (Miltenyi Biotec), per the manufacturer's Extraction Kit protocol, with the following modifications: a) protease inhibition buffer was prepared with 100× HALT protease inhibitor cocktail (Thermo Fisher); b) minced tissue was resuspended with an increased protease inhibitor buffer volume of 2×2.5 mL instead of 2×1 mL; c) after homogenization, the homogenate was passed through a 30 um SmartStrainer (Miltenyi Biotec); d) the SmartStrainer was washed with 2×2.5 mL solution 3 instead of 2×1 mL; e) prior to adding TOM22 antibody, the homogenate was diluted with Separation Buffer to a volume of 25 mL instead of 10 mL; and, f) 125 uL TOM22 antibody was used per sample instead of 50 uL. Final mitochondria pellets were frozen at −20° C. for subsequent DNA extraction

### DNA Extraction

The DNA extraction method used for each sample is listed in Supplementary Table 1. Below are details of each DNA extraction method.

### Sperm for HiDEF-Seq

An aliquot of washed sperm (i.e., after the washes that are performed after density gradient centrifugation) was centrifuged at 300×g for 5 minutes at room temperature. The supernatant was removed, leaving approximately 50 μL of sperm/buffer at the bottom of the microtube. The tube was tapped gently 5 times to break up the sperm pellet before adding lysis buffer.

If starting with frozen sperm instead of an aliquot of washed sperm, the frozen sperm vial is rapidly thawed in a 37° C. water bath, gently pipette mixed, and an aliquot is transferred to a 2 mL DNA LoBind microtube for DNA extraction. The remaining sperm is frozen again. The DNA extraction aliquot is diluted with 600 μL of Origio sperm wash buffer, centrifuged at 300×g for 5 minutes at room temperature, and the supernatant is removed to leave approximately 100 μL of sperm/buffer at the bottom. The sperm is diluted again with 600 μL of Origio sperm wash buffer, centrifuged at 300×g for 5 minutes at room temperature, and the supernatant is removed to leave approximately 50 μL of sperm/buffer at the bottom. The tube was tapped gently 5 times to break up the sperm pellet before adding lysis buffer. Sperm DNA extraction is based on ref.⁸⁷, with some modifications, including optimizations we performed that showed that TCEP can be reduced from 50 mM to 2.5 mM in the lysis buffer. Sperm lysis buffer was prepared by combining (for each sample) 497.5 μl of Qiagen Buffer RLT (Qiagen) without beta-mercaptoethanol, and 2.5 μl of 0.5M Bond-Breaker TCEP Solution (Thermo Scientific) for a lysis buffer with 2.5 mM TCEP final concentration. 500 μl of sperm lysis buffer was added to each sample, without pipette mixing. 100 mg of 0.2 mm stainless steel beads (Next Advance, SSB02) were then added to each sample and homogenization was performed with the TissueLyser II (Qiagen) at 20 Hz for 4 minutes (samples SPM-1002, SPM-1013, SPM-1020, SPM-1004) or 30 seconds (sample SPM-1060). DNA was then extracted using the QIAamp DNA Mini Kit (Qiagen) with a modified protocol as follows. 500 μl of buffer AL was added to each lysate and vortexed well. Then, 500 μl of 100% ethanol was added and vortexed well. Then, the mixture was applied to a QIAamp DNA Mini spin column and the remaining standard QlAamp protocol was followed. DNA was eluted with 100 μl of 10 mM Tris pH 8. RNase treatment was then performed by adding 12 μL of 10× PBS pH 7.4 (Gibco), 2 μL of Monarch RNase A (New England Biolabs (NEB)), and 6 μL nuclease-free water (NFW). The reaction was incubated at room temperature for 5 mins and immediately purified using SPRI beads (0.8× beads:reaction ratio) with elution using 35 μL of 10 mM Tris/0.1 mM EDTA pH 8.

A somatic cell contamination assay was adapted from ref.⁸⁸ and performed on all extracted sperm DNA samples to further confirm sperm purity. This assay amplifies 4 loci from bisulfite-treated DNA: 3 loci that are methylated in sperm but not in somatic cells (PCR7, PCR11, PCR31), and 1 locus that is methylated in somatic cells but not in sperm (PCR12). Following bisulfite treatment and PCR amplification of each locus, the PCR amplicon is only cut by a restriction enzyme if the original DNA was methylated. Therefore, this assay can detect somatic cell contamination. 350 ng of each extracted sperm DNA and 350 ng of control human NA12878 genomic DNA (Coriell Institute) were bisulfite-converted using the Zymo EZ DNA Methylation kit (Zymo Research). The loci were amplified by PCR using the following primer sets: PCR7 (GGGTTATATGATAGTTTATAGGGTTATT (SEQ ID NO:8)) and TCTATTACTACCACTTCCTAAATCAA (SEQ ID NO:1)), PCR11 (TGAGATGTTTGTTAGTTTATTATTTTGG (SEQ ID NO:2) and TCATCTTCTCCCACCAAATTTC (SEQ ID NO:3), PCR12 (TAGAGGGTAGTTTTTAAGAGGG (SEQ ID NO:4) and ATTAACCAACCTCTTCCATATTCTT (SEQ ID NO:5)), and PCR31 (TTTTAGTTTTGGGAGGGGTTGTTT (SEQ ID NO:6) and CTACCAAAATTAAAAACCAACCCAC (SEQ ID NO:7). The PCR reaction contained 1.5 μL of bisulfate converted DNA, 10 μL of 2× ZymoTaq PCR Mix (Zymo Research), PCR primers, and NFW to a final volume of 20 μL. The PCR reactions were optimized to contain the following final concentrations of each forward and reverse primer: 0.6 μM for PCR7 primers, 0.6 μM for PCR11 primers, 0.3 μM for PCR12 primers, and 0.45 μM for PCR31 primers. The PCR reactions were cycled at: 95° C. for 10 mins, (94° C. for 30 sec; X° C. for 30 sec; 72° C. for 30 sec)×40 cycles, 72° C. for 7 mins, 4° C. hold, where X (annealing temperature) was 49° C. for PCR7 and PCR11, 51° C. for PCR12, and 55° C. for PCR31. PCR reactions were purified by 2× volumetric ratio SPRI beads cleanup and eluted in 22 μL of 10 mM Tris pH 8. Restriction digests were performed by combining 5 μL of purified PCR product, restriction enzyme (10 units of HpyCH4IV [NEB] for PCR7 and PCR31, and 20 units of Taq1-v2 [NEB] for PCR11 and PCR12), 1 μL of 10× CutSmart buffer (NEB), and NFW for a total reaction volume of 10 μL. Restriction digestions were at 37° C. (HpyCH4IV) or 65° C. (Taq1-v2) for 60 mins. Negative control resctriction digest reactions (no restriction enzyme) were performed for each sample/restriction digest combination. 5 μL of each restriction digest reaction was combined with 1 μL 6× TriTrack DNA Loading Dye (Thermo Fisher) and run on a 2% agarose gel pre-stained with ethidium bromide, followed by imaging of the gel.

### Solid Tissues for HiDEF-Seq

Approximately 50-300 mg of tissue was cut in a petri dish on dry ice and minced with a scalpel, followed by one of the following DNA extraction methods, as specified in Supplementary Table 1.

‘Nucleobond HMW, MagAttract HMW, Qiaamp’: In this method, DNA was extracted and purified with 3 serial kits to maximize DNA purity. DNA was extracted using the NucleoBond HMW DNA kit (Takara) with a 50° C. proteinase K incubation for 4.5 hours. The eluted DNA was then further purified with the MagAttract HMW DNA kit (Qiagen) per the manufacturer's whole blood purification protocol, except with Proteinase K/RNase A incubation occurring at 56° C. for 20 minutes. The eluted DNA was then further purified using the QIAamp DNA mini kit (Qiagen) by diluting the DNA to a final volume of 200 μL and final 1× PBS concentration, adding 20 μL of Proteinase K (Qiagen), and continuing per the manufacturer's body fluids DNA purification protocol with a 56° C. proteinase K incubation for 10 minutes without RNase A treatment.

‘MagAttract HMW’: We used the MagAttract HMW DNA (Qiagen) per the manufacturer's protocol for tissue, with a 2 hour proteinase K digestion at 56° C. DNA was eluted with 10 mM Tris pH 8.

‘Puregene’: Tissue was pulverized inside a microtube while in a liquid-nitrogen cooled mini mortar and pestle (Bel-Art). DNA was then extracted with the Puregene DNA Kit (Qiagen) per the manufacturer's protocol for tissues, except: 1) the lysis step was performed at room temperature on a thermomixer (1,400 rpm) for 1 hour; 2) the RNaseA treatment was performed at room temperature for 20 minutes, and; 3) the final DNA pellet was resuspended at room temperature for 1 hour.

### Cerebral Cortex Neuronal Nuclei for HiDEF-Seq

DNA was extracted from nuclei pellets by two methods, as specified in Supplementary Table 1.

‘Qiamp’: We used the QIAamp DNA Mini kit (Qiagen) per the manufacturer's protocol, with lysis performed by adding 180 μL of Buffer ATL and 20 μL of proteinase K to the nuclei pellet, followed by a 56° C. incubation for 1 hour, and including RNase A treatment.

‘MagAttract’: We used the MagAttract HMW DNA kit per the manufacturer's protocol for blood, after resuspending nuclei with 200 uL of 1× PBS, with a 30 minute proteinase K digestion at room temperature.

### Mitochondria for HiDEF-Seq

DNA was extracted from mitochondria pellets with the Puregene DNA Kit (Qiagen) per the manufacturer's protocol for tissues, except: a) the lysis step used 200 uL Cell Lysis Solution and 1.5 uL Proteinase K and was performed at room temperature for 30 minutes; and, b) the RNaseA treatment was performed at room temperature for 20 minutes.

Note, due to low yields of mitochondria DNA preparations, these samples were profiled with HiDEF-seq v2 with A-tailing (see ‘HiDEF-seq library preparation’).

### Blood, Lymphoblastoid Cells, and Fibroblasts for HiDEF-Seq and Germline Reference Sequencing

DNA was extracted from all blood, lymphoblastoid cells, and fibroblasts (the latter two after resuspending cell pellets in 1× PBS) with the MagAttract HMW DNA kit (Qiagen) per the manufacturer's whole blood purification protocol, with proteinase K incubation at room temperature for 30 minutes. For the experiment excluding a measurable cytosine deamination effect by leached iron from MagAttract magnetic beads (FIG. 13d), an additional aliquot of DNA was extracted from blood of individual 1901 with the Puregene DNA Kit (Qiagen) per the manufacturer's protocol for ‘whole blood or bone marrow’, except: 1) 200 uL blood was first diluted with 100 uL of 1×PBS; b) the cell lysis step was performed at room temperature; and, c) the RNaseA treatment was performed at room temperature for 20 minutes.

### Saliva for Illumina Germline Reference Sequencing

DNA was extracted with the QIAamp DNA Mini Kit per the manufacturer's ‘DNA purification from blood or body fluids’ protocol and including RNase A treatment.

### Liver and Spleen for Illumina Germline Reference Sequencing

DNA was extracted with the QIAamp DNA Mini Kit per the manufacturer's ‘DNA purification from tissues’ protocol, with a 2 hour proteinase K digestion at 56° ° C. and including RNase A treatment.

### Blood for Pacific Biosciences Germline Reference Sequencing

DNA was extracted using the Chemagic DNA Blood 2k Kit (Perkin Elmer CMG-1097) on a Chemagic 360 automated nucleic extraction instrument (Perkin Elmer) following manufacturer's protocols for DNA isolation from whole blood.

### DNA Quantity and Quality Measurements, and Storage

Concentration and quality of all DNA samples was measured using a NanoDrop (Thermo Fisher), Qubit 1× dsDNA HS Assay Kit (Thermo Fisher), and Genomic DNA ScreenTape TapeStation Assay (Agilent). DNA was stored at −20° C.

### Illumina Germline Reference Library Preparation and Sequencing

Libraries were prepared using the TruSeq DNA PCR-Free kit (Illumina) for all samples, except GM10430 that used the TruSeq DNA Nano kit (Illumina). At least 110 Gb (˜40× genome coverage) of 150-base pair paired-end sequencing per sample was performed on a Novaseq 6000 instrument (Illumina) by Psomagen, Inc.

### Pacific Biosciences Germline Reference Library Preparation and Sequencing

15 μg of DNA was cleaned with a 1× AMPure PB beads cleanup and sheared to a target size of 14 kb using the Diagenode Megaruptor 3 with the following settings: Speed 36, vol 300 uL, Conc. 33 ng/uL. Library preparation was performed with the Pacific Biosciences SMRTbell Express Template Prep Kit 2.0 following the manufacturer's standard protocol. Fragments longer than 10 kb were selected using the Sage Science PippinHT instrument. Size-selected libraries were sequenced on a Pacific Biosciences Sequel IIe system using the Sequel II Binding Kit 2.0 and Sequel II Sequencing Kit 2.0 (Pacific Biosciences), Sequencing primer v4, 2 hour binding time, adaptive loading, 2 hour pre-extension, and 30 hour movies.

### Heat Damage of DNA

The amount of input DNA for the subsequent HiDEF-seq library was heated in a volume of 62 uL at the specified temperature, for the specified time, and in the specified buffers listed in Supplementary Table 1, followed by incubation on ice up to a total of 6 hours if the heating time was less than 6 hours. Untreated samples were incubated on ice for 6 hours. The DNA was then input into HiDEF-seq library preparation.

### NanoSeq Library Preparation and Sequencing

NanoSeq libraries were prepared as previously described¹⁷ with 50 ng DNA input from the same aliquots used for HiDEF-seq.

### HiDEF-Seq Library Preparation and Sequencing

### Choice of Restriction Enzymes for DNA Fragmentation

We performed in silico digests of the CHM13 v1.0 human reference genome⁸⁹ to identify restriction enzymes that: a) maximize the percentage of the genome between 1-4 kilobases (kb), b) are active at 37° C., and, c) the DNA is fragmented with blunt ends, since blunt fragmentation avoids single-strand overhangs that can lead to artifactual double-strand mutations during end repair¹⁷. This in silico digest screen identified Hpy166II (5′-GTN/NAC-3′ recognition sequence) as the optimal restriction enzyme, with a prediction of 37% of the genome mass fragmenting between 1-4 kb. The percentage of the genome fragmented to sizes between 1-4 kb was then empirically measured by fragmenting 1 μg of genomic DNA followed by quantification on a Genomic DNA ScreenTape assay (Agilent). Hpy166II fragments 41% of the genome to within the target size range. Note that although Hpy166II is blocked by methylated CpG when present on both sides of the recognition sequence (New England BioLabs), this will occur only with the larger recognition sequence 5′-C*GTN/NAC*G-3′ (′*′ signifies methylation of preceding cytosine); excluding all these potential bi-methylated sites actually increases the in silico predicted percentage of the genome fragmented by Hpy166II to within the target size range by 0.2%, and 99.97% of genomic bases within the original target size range remain when excluding these as potential fragmentation sites.

For the mitochondrial genome, Hpy166II captures 3 fragments in our target 1-4 kb size range, at the following coordinates (CHM13 v1.0): 1) 3068-5116 (2048 bp), 2) 7581-9439 (1858 bp), and, 3) 10441-11831 (1390 bp). These encompass 32% of the mitochondrial genome.

### HIDEF-Seq Library Preparation

Input DNA amounts of 500-3000 ng (as measured by Qubit) were used per library, depending on available DNA. With high-quality DNA and HiDEF-seq version 2 (i.e., with nick ligation, see below), input amounts of 500 ng provide sufficient library yield for approximately one full (non-multiplexed) Pacific Biosciences (PacBio) Sequel II instrument sequencing run, and lower input amounts are feasible for filling a fraction of a sequencing run; we have successfully made libraries with as low as 200 ng input DNA, producing sufficient yield for 40% of a sequencing run. For fragmented DNA samples, more than 1500 ng of input DNA is generally required. Generally, for samples other than sperm that have low mutation burdens, one quarter of a sequencing run is sufficient for mutation burden and pattern analysis. Input DNA A260/A280>1.8 and A260/A230>2.0 absorption ratios were confirmed on NanoDrop prior to library preparation per the Pacific BioSciences DNA preparation guidelines.

Input DNA was fragmented with 0.14 U/uL of Hpy166II restriction enzyme (NEB) in a 70 μl reaction with 1× CutSmart buffer (NEB) for 20 minutes at 37ºC. The reaction was scaled to 90 μL if the input DNA was too dilute to accommodate a 70 μL reaction. After the reaction was complete, DNA was quantified using a Qubit 1× dsDNA HS Assay.

We found that effective removal of <1 kilobase DNA fragments with high-yield recovery of larger DNA requires two tandem AMPure PB bead (Pacific Biosciences) size selections with an optimized dilution of AMPure PB beads after the restriction digest and a third AMPure PB bead size selection after A-tailing, and that it also critically depends on a DNA concentration<10 ng/μl in each bead purification. First, AMPure PB Beads were diluted with Elution Buffer (Pacific Biosciences) to a final 75% bead volume/total volume solution; these diluted beads were used for all AMPure bead purifications and size selections. Next, the restriction enzyme reaction was diluted with NFW to a DNA concentration of 10 ng/μl based on the pre-digest Qubit concentration. For the first bead purification, a ratio of 0.8× diluted AMPure beads to sample volume was used, with two 80% ethanol washes, and the DNA was eluted with 22 μL of 10 mM Tris pH 8. The DNA concentration was measured again with the Qubit 1× dsDNA HS Assay.

For HiDEF-seq v2 (but not HiDEF-seq v1), nick ligation was then performed in a 30 μL reaction with 3 μL of 10× rCutSmart Buffer (NEB), 1.56 μL of 500 UM β-Nicotinamide adenine dinucleotide (NAD+) (NEB), and 15U of E. coli DNA Ligase (NEB). The nick ligation reaction was incubated at 16° C. for 30 minutes with the heated lid turned off.

The DNA was then diluted with 10 mM Tris pH 8 to 10 ng/μL (or not diluted if DNA is already less than 10 ng/μl) based on the prior Qubit concentration. For the second bead purification, a ratio of 0.75× diluted AMPure beads to sample volume was used, with two 80% ethanol washes, and the DNA was eluted with 22 μL of 10 mM Tris pH 8. DNA concentration was measured with the Qubit 1× dsDNA HS Assay.

The DNA was then A-tailed as in ref. 17 in 30 μL volume reactions with 20 μL of input DNA, 2.5 μL NFW, 3 μL 10× NEBuffer 4 (NEB), 3 μL containing 1 mM each dATP/ddBTP (ddBTP=mix of 1 mM each ddCTP, ddTTP, ddGTP; Jena Bioscience, NU-1019S), and 7.5 U of Klenow fragment 3′→5′ exo-(i.e., 1.5 μL of 5 U/μL enzyme) (NEB). For fragmented DNA (e.g., post-mortem kidney and liver), this reaction was performed without dATP. The reaction was incubated at 37° C. for 30 minutes. Next, a third size selection was performed: the reaction volume was diluted with 10 mM Tris pH 8 to 10 ng/μL DNA based on the Qubit concentration measured for the DNA prior to the reaction, followed by a ratio of 0.75× diluted AMPure beads to sample volume, with two 80% ethanol washes, and elution of DNA with 22 μl of 10 mM Tris pH 8. The eluted DNA was then adjusted to a total of 30 μL with 3 μL of 10× NEBuffer 4 and NFW before proceeding to adapter ligation.

PacBio adapter ligation was performed using reagents from the SMRTbell Express Template Prep Kit 2.0 (Pacific Biosciences) in a volume of 48.5 μL containing 30 μL of DNA, 2.5 μL Barcoded Overhang Adapter (Pacific Biosciences), 15 μL Ligation Mix, 0.5 μL Ligation Additive, and 0.5 μL of Ligation Enhancer. For samples whose preceding Klenow polymerase reaction was performed without dATP, 2.5 μL of 17 uM annealed blunt adapters were used instead (see sequences and preparation below). The adapter ligation reaction was incubated at 20° C. for 60 minutes with the heated lid turned off. Immediately after the adapter ligation, nuclease treatment was performed using the SMRTbell Enzyme Clean Up Kit 1.0 (Pacific Biosciences) to remove any non-circularized DNA containing nicks and/or without hairpin adapters: 2 μL Enzyme A, 0.5 L Enzyme B, 0.5 μL Enzyme C, and 1 μL Enzyme D were combined, and 4 μL of this enzyme mix was added to the ligation reaction and incubated at 37° C. for 60 minutes. After the nuclease treatment, samples were purified with a ratio of 1.2× diluted AMPure beads to sample volume and eluted with 24 μL of 10 mM Tris pH 8.

After the post-ligation AMPure bead purification, non-A tailed HiDEF-seq v2 libraries underwent an additional 1.1× diluted AMPure bead purification to remove residual adapter dimers.

Final library concentration and size distribution were measured with Qubit 1× dsDNA HS Assay and High Sensitivity D5000 ScreenTape. The final library fragment size distribution should contain <5% of DNA mass<1 kilobase and <5% of DNA mass>4 kilobase (percentages calculated using the ScreenTape analysis software's manual region analysis ‘% of Total’ field). Final library mass yield should be ˜6-10% of the input genomic DNA mass. Libraries were stored in 0.5 mL DNA LoBind tubes at −20° C.

On ScreenTape, some non-A tailed HiDEF-seq v2 libraries may have a low level of residual adapter dimers, which can be removed with a final 1.3× diluted AMPure bead cleanup after multiplexing the libraries from the same run (see multiplexing details in the ‘HIDEF-seq library sequencing’ section).

Sequences and Preparation of Blunt Adapters Used for HiDEF-Seq without A-Tailing

bcAd1001:
(SEQ ID NO: 9)
/5′Phos/ACGCACTCTGATATGTGATCTCTCTCTTTTCCTCCTCCTCCGTTGTTGTTGTTGA
GAGAGATCACATATCAGAGTGCGT (barcode = CACATATCAGAGTGCG
(SEQ ID NO: 10)
bcAd1002:
(SEQ ID NO: 11)
/5′Phos/ACTCACAGTCTGTGTGTATCTCTCTCTTTTCCTCCTCCTCCGTTGTTGTTGTTGA
GAGAGATACACACAGACTGTGAGT (barcode = ACACACAGACTGTGAG
(SEQ ID NO: 12)
bcAd1003:
(SEQ ID NO: 13)
/5′Phos/ACTCTCACGAGATGTGTATCTCTCTCTTTTCCTCCTCCTCCGTTGTTGTTGTTGA
GAGAGATACACATCTCGTGAGAGT (barcode = ACACATCTCGTGAGAG
(SEQ ID NO: 14)
bcAd1008:
(SEQ ID NO: 15)
/5′Phos/ACGCAGCGCTCGACTGTATCTCTCTCTTTTCCTCCTCCTCCGTTGTTGTTGTTG
AGAGAGATACAGTCGAGCGCTGCGT (barcode =
ACAGTCGAGCGCTGCG (SEQ ID NO: 16))

Adapters (4 different barcoded sequences above) were ordered as HPLC-purified oligonucleotides from Integrated DNA Technologies. Each adapter was reconstituted to 100 uM concentration with nuclease-free water. Annealing was then performed for each adapter at a concentration of 17 uM in a 30 μL volume containing 10 mM Tris pH 8 and 50 mM NaCl, by incubating at 95° C. for 30 minutes and cooling at room temperate for 30 minutes. Additional barcoded adapters can be designed by replacing the above barcodes with alternative sequences.

Modified HiDEF-Seq Library Preparation Trials to Remove ssDNA T>A Artifacts

Below are details of initial attempts to remove ssDNA T>A artifacts arising from residual ssDNA nicks. The final protocol that completely removes these artifacts (HiDEF-seq v2 without A-tailing) is described in the main ‘HIDEF-seq library preparation’ section.

Polynucleotide kinase: The standard HIDEF-seq v2 protocol was followed with the exception that prior to nick ligation, the DNA was treated in a 30 μL reaction containing 12 U T4 polynucleotide kinase (NEB), 1 mM ATP, 4 mM DTT, and 1× CutSmart buffer (NEB) at 37° C. for 1 hour. The sample then proceeded into the nick ligation reaction in an increased reaction volume of 35 μL, with reaction components scaled proportionally to the higher volume and a final 1× CutSmart buffer concentration.

Alternative A-tailing polymerases: The standard HiDEF-seq v2 protocol was followed with the exception of replacing Klenow fragment 3′→5′ exo-polymerase with one of the following: 9.6 U Bst large fragment (NEB), 9.6 U Bst 2.0 (NEB), 9.6 U Bst 3.0 (NEB), or 9 U Isopol SD+(ArcticZymes). Reaction temperatures and times were: 30 minutes at 45° C. for Bst large fragment and Bst 2.0, either 30 minutes or 150 minutes at 45° C. or 210 minutes at 37° C. for Bst 3.0, and either 30 minutes or 210 minutes at 37° C. for Isopol SD+.

Pyrophosphatase: The standard HiDEF-seq v2 protocol was followed with the exception of adding 0.15 U E. Coli inorganic pyrophosphatase (NEB).

Klenow polymerase reaction without dATP or ddBTP: The standard HiDEF-seq v2 protocol was followed with the exception that the Klenow polymerase reaction was performed without dATP or ddBTP.

No Klenow polymerase reaction: The standard HiDEF-seq v2 protocol was followed, except after the post-nick ligation AMPure bead purification, the DNA was diluted to 30 μl in a final 1× NEBuffer 4 concentration and taken directly to adapter ligation using blunt adapters. Following the post-ligation AMPure bead purification, an additional size selection with 0.75× diluted AMPure beads was performed since this would ordinarily have occurred after the Klenow polymerase reaction. Note: this protocol produces a CCT>CGT ssDNA artifact that does not occur when the Klenow polymerase reaction is performed without dATP or ddBTP, indicating that Klenow polymerase removes this artifact likely via a pyrophosphorolysis mechanism.

### HiDEF-Seq Large Fragment Library Preparation

Large fragment size libraries (1-10 kilobase range; median 4.1 kilobase size) were prepared per the above HiDEF-seq library protocol, except: 1) fragmentation was performed with 30 U Pvull-HF enzyme (NEB) instead of Hpy166II, 2) post nick ligation and post A-tailing cleanups were performed with 1.8× undiluted AMPure PB beads, and DNA was not diluted to <10 ng/μL (since size selection is not being performed), and, 3) final post-nuclease treatment cleanup was performed with 1× undiluted AMPure PB beads.

HiDEF-Seq Library Preparation with Random Fragmentation

Libraries were prepared per the above HiDEF-seq library protocol without A-tailing (i.e., Klenow reaction without dATP and utilizing blunt adapters), except: 1) A higher amount of input DNA was used: 4 μg per sample; 2) Instead of restriction enzyme fragmentation, DNA was acoustically fragmented in miniTUBE Clear tubes (2 μg per tube, i.e., 2×2 μg aliquots per sample), with each 2 μg DNA aliquot diluted to 200 μl in a final buffer of 10 mM Tris pH 8 and 50 mM NaCl, on an ME220 instrument (Covaris) with the following settings: temperature 7° C., treatment time 900 seconds, peak incident factor 8 W, duty factor 20%, and cycles/burst 1000; 3) Each 2 μg fragmented DNA aliquot was blunted in a 200 μL reaction containing 0.5 U/μL Nuclease P1 (NEB) and 1× NEBuffer r1.1 (NEB) at 37° C. for 30 minutes, after which the reaction was stopped by adding 8 μL of 0.5 M EDTA and 2 μL of 1% SDS; 4) Following the Nuclease P1 reaction, the protocol continued with the 0.8× diluted AMPure bead purification as is usually performed after restriction enzyme fragmentation, and the two aliquots of each sample were combined at the elution stage for a final elution volume of 22 μL; 5) Prior to nick ligation, the DNA was treated with 0.4 U/μL T4 polynucleotide kinase (NEB), 1 mM ATP, and 4 mM DTT in a 30 μL volume of 1× rCutSmart buffer (NEB) at 37° C. for 1 hour; 6) Nick ligation was performed immediately after by adding the required reagents to the T4 polynucleotide kinase reaction to a final volume of 35 μL. 7) The bead purification after the Klenow reaction was performed with a 1.2× ratio of diluted AMPure bead volume to sample volume, instead of a ratio of 0.75×; 9) After nuclease treatment, libraries underwent a 1.2× diluted AMPure bead purification, then libraries for the same sequencing run were pooled, and a final 1.0× diluted AMPure bead purification was performed to remove residual adapter dimers.

### HIDEF-Seq Library Sequencing

Libraries sequenced on the same sequencing run were multiplexed together based on the final library Qubit quantification, to achieve at least 50 ng of total library in no more than 15 μL volume. When necessary, the concentration of individual or pooled libraries can be increased by room temperature centrifugal vacuum concentration (Eppendorf Vacufuge) and pausing periodically (approximately every 3 minutes) to avoid increases in temperature, or with an AMPure PB bead clean-up.

Sequencing was performed on Pacific Biosciences Sequel II or Sequel IIe systems with 8M SMRT Cells by the Icahn School of Medicine at Mount Sinai Genomics Core Facility and the New York University Grossman School of Medicine Genome Technology Center. Sequencing parameters were: Sequel II Binding Kit 2.0 and Sequel II Sequencing Kit 2.0 (Pacific Biosciences), Sequencing primer v4, 1 hour binding time, diffusion loading, loading concentrations between 125-160 pM (lower concentration was used for blood than for tissues) for standard size libraries (Hpy166II libraries) or 80 pM for large fragment libraries (Pvull libraries), 2 hour pre-extension, and 30 hour movies.

### Germline Reference Sequencing Data Processing

### Illumina Germline Reference Data Processing

Reads were aligned to the CHM13 v1.0 reference genome⁸⁹ with BWA-MEM v0.7.1790 with standard settings, followed by marking of optical duplicates and sorting using the Picard Toolkit⁹¹. Variants were called from the aligned reads with two different variant callers: 1) Genome Analysis Toolkit (GATK)⁹² v4.1.9.0 using the HaplotypeCaller tool with parameters ‘-ERC GVCF-G StandardAnnotation-G StandardHCAnnotation-G AS_StandardAnnotation’ followed by the GenotypeGVCFs tool with default parameters; 2) DeepVariant⁹³ v1.2.0 with parameter: ‘-model_type=WGS’. Both GATK and DeepVariant variant calls are used during subsequent mutation analysis.

### Pacific Biosciences Germline Reference Data Processing

Circular consensus sequences were derived from raw subreads (a subread is one sequencing pass of a single strand of a DNA molecule) using pbccs (ccs, Pacific Biosciences) with default parameters, and consensus sequences were filtered to only retain high-quality “HiFi” reads with the predicted consensus accuracy ‘rq’ tag≥0.99 (‘rq’ is calculated by ccs as the average of the per base consensus qualities of the read). These reads were then aligned to the CHM13 v1.0 reference genome with pbmm2 (Pacific Biosciences) with the parameters ‘-preset CCS-sort’. Variants were called from the aligned reads with DeepVariant⁹³ v1.2.0 with the parameter: ‘-model_type=PACBIO’.

### HiDEF-Seq Primary (Raw) Data Processing

HIDEF-seq raw data is first processed via a two-part computational pipeline that transforms the raw data into a format suitable for mutation analysis. Primary data processing also includes quality control plots generated by custom scripts and using the SMRT Link (Pacific Biosciences) software to assess each sequencing run's quality (e.g. distributions of polymerase read lengths, number of passes, etc.). Note that in this Methods section, for simplicity, we sometimes use the term ‘mutation’ to refer also to ssDNA calls even though these include both ssDNA mismatches and ssDNA damage.

The first part of the raw data processing pipeline utilizes a combination of bash and awk scripts to process raw subread sequencing data (a subread is one sequencing pass of a single strand of a DNA molecule) into a strand-specific aligned BAM format with additional tags needed for mutation analysis⁹⁴, as follows:
1) Subreads for which an adapter was not detected on both ends of the molecule (cx tag not equal to 3) are removed.
2) Consensus sequences are created separately for each strand of the DNA molecule (i.e. forward and reverse strand separately) using pbccs version 6.2.0 (Pacific Biosciences) with parameters: -by-strand, -min-rq 0.99 (predicted read consensus accuracy >Q20 to remove low quality consensus sequences), and -top-passes 0 (unlimited number of full-length subreads used per consensus).
3) Demultiplexing of samples according to adapter barcodes using lima version 2.5.0 (Pacific Biosciences) with parameters: -ccs-same-split-named-min-score 80-min-end-score 50-min-ref-span 0.75-min-scoring-regions 2.
4) Filter to remove any DNA molecules (also known as zero-mode waveguides, ZMWs, which are sequencing wells containing a single DNA molecule) that did not successfully produce both one forward and one reverse strand consensus sequence, since we are only interested in molecules that produce data for both strands.
5) Align forward and reverse strand consensus sequences to the CHM13 v1.0 reference genome⁸⁹ using pbmm2 v1.7.0 (Pacific Biosciences), an aligner based on minimap2⁹⁵, with parameters: -preset CCS. Note that the CHM13 v1.0 reference genome only contains nuclear chromosomes 1-22 and chromosome X, and the mitochondrial genome—but not chromosome Y, which is therefore not part of the analyses.
6) Filter to only keep DNA molecules that produce only 1 forward strand primary not-supplementary alignment and 1 reverse strand primary not-supplementary alignment, where the forward and reverse alignments overlap (reciprocally) in the genome by at least 90%.
7) Sort alignments by reference position.
8) Add five comma-separated tag arrays to each alignment of the final BAM file (each molecule has one forward strand and one reverse strand alignment), with all the arrays of each alignment having the same size corresponding to the number of base mismatches in the alignment (i.e., bases in the alignment that do not match the reference genome, based on the alignment CIGAR string):
   qp: Positions of bases in query (alignment) that are mismatches relative to the genome reference; 1-based coordinates with the left-most query base as represented in the alignment record's SEQ column=1.
   qn: Sequences of bases in the query that are mismatches relative to the genome reference (base sequences are according to the forward genomic strand, i.e., they are taken from the SEQ column of the SAM alignment record).
   qq: Qualities of bases in the query that are mismatches relative to the genome reference (taken from the QUAL column of the SAM alignment record).
   rp: Positions in reference genome coordinates of query bases that are mismatches relative to the reference genome.
   rn: Sequences of the reference genome at positions of query base mismatches.

The second part of the raw data processing pipeline is an R⁹⁶ script (R v4.1.2, requiring the packages Rsamtools⁹⁷, GenomicAlignments⁹⁸, GenomicRanges⁹⁸, vcfR⁹⁹, plyr¹⁰⁰, configr¹⁰¹, qs¹⁰²) that further processes and annotates the aligned BAM file into an R data file, as follows:
1) Load the aligned BAM file into R, including the custom tags that annotated the positions of all base mismatches relative to the human reference genome.
2) Annotate variants (bases differing from the reference genome) for which the reference genome base is ‘N’, to exclude these from subsequent analyses.
3) Annotate the positions of insertions and deletions in each alignment, based on the alignment CIGAR string.
4) Annotate each variant if it was present in any of the VCF variant call files of the corresponding individual's germline reference sequencing, along with details of the VCF variant annotation.
5) Save positions of insertions and deletions from the VCF variant call files of the corresponding individual's germline reference sequencing.
6) Transform the dataset so that forward and reverse strand consensus reads and ssDNA and dsDNA variants (and tag information) from the same DNA molecule are linked to each other as dsDNA molecules.
7) Save the final R dataset to a file.

### HiDEF-Seq Call Filtering

The call filtering analysis for single-base substitutions (SBSs) implements a series of filters that were optimized to maximize the number of true SBS calls identified while minimizing the number of sequenced bases and regions of the genome that are filtered out. The specific filters and filter parameters used in the pipeline were determined by iterative adjustments in filters and filter parameters followed by manual examination in the Integrative Genomics Viewer (IGV)¹⁰³ of somatic mutations identified in low mutation rate samples (tissues from infants and sperm) to identify false positives. These false positives were apparent by a variety of pieces of evidence, mainly due to clusters of mutations in low-quality regions of the genome and/or low-quality regions of sequencing reads. For example, when a metric of low-quality genome regions was found to correlate with clusters of candidate somatic mutations, this metric was added as a filter, and its threshold was iteratively tuned to maximally remove all false positives while minimizing the number of sequenced bases and regions of the genome that are filtered out.

Additional optimization of filter thresholds was performed using sperm samples that have a known low double-strand DNA (dsDNA) SBS burden. Specifically, we plotted the dsDNA and ssDNA SBS burdens with varying: 1) minimum predicted consensus accuracy (0.99 to 0.999), 2) minimum number of passes per strand (5 to 20), and, 3) minimum fraction of subreads (passes) detecting the mutation (0.5 to 0.8). See below sections for details of each of these filters. We examined these plots for threshold settings above which burden estimates are stable; since each of these filters is adjusted by sensitivity factors (based on total interrogated bases and detection of known germline variants), a decrease in burden estimates with increasing threshold settings indicates removal of sequencing artifacts. These plots showed that sperm dsDNA mutation burden estimates were stable even down to the lowest thresholds (FIGS. 8d,e,g). In contrast, ssDNA mismatch burden estimates required higher threshold settings for each of these filters before burden estimates stabilized (FIGS. 8i,j). Individually increasing the thresholds of each of the above three filters stabilized ssDNA burden estimates at approximately 20%, 15%, and 10% lower levels, respectively, compared to the least stringent settings, and applying all three filters together with these higher thresholds reduced the ssDNA burden estimate by approximately 25% (i.e., the three filters are not independent). Specific thresholds used for dsDNA and ssDNA mismatch filtering are detailed in the below sections detailing each filter.

The mutation analysis pipeline utilizes the following R packages: GenomicAlignments⁹⁸, GenomicRanges⁹⁸, vcfR⁹⁹, Rsamtools⁹⁷, plyr¹⁰⁰, configr¹⁰¹, MutationalPatterns¹⁰⁴, magrittr¹⁰⁵, readr¹⁰⁶, dplyr¹⁰⁷, plyranges¹⁰⁸, stringr¹⁰⁹, rtracklayer¹¹⁰, qs¹⁰²; and the following software tools: bcftools¹¹¹, samtools¹¹¹, wigToBigWig¹¹², wiggletools¹¹³, pbmm² (Pacific Biosciences), zmwfilter (Pacific Biosciences), digest¹¹⁴ SeqKit¹¹⁵, and KMC¹¹⁶.

Additional filters used in the pipeline were created using REAPR v1.0.18¹¹⁷. REAPR was originally designed to identify regions with errors in genome reference assemblies, but it also calculates metrics useful for identifying regions of the genome prone to generating false positive and false negative variant calls due to Illumina short read data. First, Illumina whole-genome sequencing reads from sperm sample 1542 were aligned to CHM13 v1.0 using SMALT v0.7.6¹¹⁸ with parameters ‘-r 0-x-y 0.5’ and a CHM13 v1.0 index created with SMALT using parameters ‘-k 13-s 2’. Next, reads were sorted and duplicates marked. The REAPR perfect from am command was then run on the resulting BAM file using the parameters ‘min insert=266, max insert=998, repetitive max qual=3, perfect min qual=4, and perfect min alignment score=151’ (min and max insert size are the 1 and 99% iles of insert sizes calculated from the sequencing data using the Picard Toolkit CollectInsertSizeMetrics tool). REAPR metrics for each base of the genome were obtained from the output stats.per_base file and a bigwig annotation file was created for each metric.

The mutation analysis filters were applied serially as described below. Unless otherwise specified, the filters were applied to both ssDNA and dsDNA SBSs. Note: the computational pipeline has the capability to implement additional filters not listed here, as specified in the pipeline configuration documentation available online.

### Filters Based on DNA Molecule Quality and Alignment Metrics

Keep only DNA molecules that meet all of the below criteria:
1) Both forward and reverse strand ccs predicted consensus accuracy ≥Q20 (i.e. rq tag of ccs≥0.99) for dsDNA mutation analysis, and ≥Q30 (i.e. rq tag of ccs≥0.999) for ssDNA mismatch analysis.
2) Minimum of 5 (for dsDNA mutation analysis) and 20 (for ssDNA mismatch and damage analysis) sequencing passes each for the forward and reverse strands (using the ‘ec’ BAM file tag, which is computed by the ccs consensus calling tool as the average subread coverage across all consensus calling windows).
3) Both forward and reverse strand mapping (i.e. alignment) quality ≥60.
4) Maximum difference in number of SBSs between the forward and reverse strands of 5, before germline VCF filtering. This removes artifacts from rare chimeric molecules and residual low-quality molecules.
5) Maximum of 20 dsDNA indels (i.e. called in both strands) relative to the human reference genome, before VCF germline filtering. This removes low-quality molecules with many indels.
6) Average number of forward and reverse strand soft-clipped bases<30. This removes low-quality molecules and molecules that align to complex regions of the genome that lead to long stretches of mismatched bases.

### Filters Based on Germline Reference Variant Calls

1) Filter out SBSs that were called in any of the individual's germline reference VCF files with read depth≥3, allele quality (QUAL column in VCF)≥3, genotype quality (GQ tag in VCF)≥3, and variant allele frequency≥0.05. This filter is applied to both ssDNA and dsDNA SBSs, since it is more likely that a ssDNA SBS in HiDEF-seq data that is at the site of a true germline SBS is due to a missed call of the HiDEF-seq SBS on the other strand rather than a germline SBS that mutated back to the human reference sequence on only one strand.
2) Filter out DNA molecules that have more than 8 dsDNA SBSs (i.e. SBSs called in both strands) remaining after VCF germline filtering. This removes molecules with misalignment to complex regions of the genome leading to many mismatches and regions of the genome for which Illumina short reads are not effective in identifying and filtering out germline variants.

### Filters Based on Genomic Regions

A. Filters that remove the entire DNA molecule if it meets any of the below criteria:
   1) For analyses using either Illumina or PacBio germline reference data:
      i) Segmental duplication regions: any overlap with the DNA molecule's forward or reverse consensus sequence alignments. This annotation was obtained from the file chm13.draft_v1.0_plus38Y.SDs.bed created by the Telomere-to-Telomere consortium³⁰. However, for analysis of mitochondrial mutations, this region filter is not used because it contains the region chrM: 10000-14910 due to a similar nuclear genome sequence on chromosome 5, which would cause unnecessary filtering of reads aligning to this region of the mitochondrial genome; note, there is negligible risk of nuclear genome sequences falsely aligning to this mitochondrial region since we obtain long reads, we require high mapping quality and exclude reads with many mismatches, and these mitochondrial and nuclear genome regions are only 94% similar.
      ii) Satellite sequence regions: ≥20% of the DNA molecule's forward and reverse strand consensus alignments (average for the two strands) overlaps the region. The satellite sequence region annotation was created using RepeatMasker v4.1.1¹¹⁹ with parameters ‘-pa 4-e rmblast-species human-html-gff-nolow’, followed by extraction of ‘Satellite’ regions.
   2) Only for analyses that use Illumina germline reference data, because short-read data is more prone to missing true germline variants in these regions:
      i) Telomere regions: any overlap with the DNA molecule's forward or reverse consensus sequence alignments. This annotation was obtained from the file chm13.draft_v1.0.telomere created by the Telomere-to-Telomere consortium⁸⁹.
      ii) 50-mer mappability score: ≥30% of the DNA molecule's forward and reverse strand consensus alignments (average for the two strands) has a mappability score<0.4. This annotation was created using Umap v1.2.0¹²⁰. This annotation calculates mappability for every base in the genome as [the number of k-mers overlapping the base that are uniquely mappable to the genome]/k.
      iii) Fraction of Illumina short reads aligning to the region that are orphaned reads (i.e., the read's mate is either unmapped or mapped to a different chromosome), averaged across the genome in 20 base pair non-overlapping bins, is ≥0.15 for ≥ 20% of the DNA molecule's forward and reverse strand consensus alignments (average for the two strands). The fraction of orphaned reads metric used in this filter is the average of the orphan_cov and orphan_cov_r REAPR metrics, which are the fraction of forward and reverse strand reads that are orphaned, respectively.
B. Filters that remove only the portions of the DNA molecule that overlap any of the following regions, while the remaining bases of the DNA molecule are still included in mutation rate analyses:
   1) Regions of the reference genome whose sequence is ‘N’.
   2) For analyses using either Illumina or PacBio germline reference data:
      i) Satellite sequence regions: any base that overlaps one of these regions.
      ii) Bases with gnomAD v3.1.2¹²¹ single nucleotide variants with ‘PASS’ flag and population allele frequency>0.1%, lifted over from the hg38 to the CHM13 v1.0 reference genome. This removes 27,476,828 genome bases from the analysis. This filter removes any residual germline variants that were not detected in the germline reference sequencing of the individual, and it reduces the risk of very low-level contamination that may have occurred among samples of the project¹⁷.
   3) Only for analyses that use Illumina germline reference data, because short-read data is more prone to missing true germline variants in these regions:
      i) 100-mer mappability score: any base with a mappability score<0.95, with mappability scores averaged across the genome in 20 base pair non-overlapping bins (binning smoothes the mappability score signal). This annotation was created as described for the above 50-mer mappability score.
      ii) Fraction of Illumina short reads aligning to the region that are properly paired (i.e., aligned in the correct orientation and within the expected distance based on insert size distribution), averaged across the genome in 20 base pair non-overlapping bins, is <0.7. The fraction of properly paired reads metric used in this filter is the average of the prop_cov and prop_cov_r REAPR metrics, which are the fraction of forward strand and reverse strand reads that are properly paired, respectively.
      iii) Fraction of Illumina short reads aligning to the region that are orphaned reads (i.e., the read's mate is either unmapped or mapped to a different chromosome), averaged across the genome in 20 base pair non-overlapping bins, is ≥0.2. The fraction of orphaned reads metric used in this filter is the average of the orphan_cov and orphan_cov_r REAPR metrics, which are the fraction of forward and reverse strand reads that are orphaned, respectively.
      iv) The number of Illumina short reads aligning to the region to either the forward or the reverse strand and that are soft-clipped at the left end or the right end (i.e. sum of REAPR clip_fl, clip_fr, clip_rl, clip_rr metrics), divided by [4×number of mapped reads/100,000,000], averaged across the genome in 200 base pair non-overlapping bins, is ≥0.09.
      v) The number of Illumina short reads with mapping quality 0 aligning to the region, divided by [4× number of mapped reads/100,000,000], averaged across the genome in 20 base pair non-overlapping bins, is ≥0.1. Note, this general filtering annotation was calculated using Illumina whole-genome sequencing data of sperm sample 1542.

### Base Quality Filter

Filter out dsDNA SBSs whose consensus sequence base quality is <93 (from QUAL column in BAM file) in either the forward or reverse strand consensus, and filter ssDNA SBSs whose base quality is <93 in the strand containing the substitution.

Filter Based on Location within the Read

Filter out SBSs that are ≤10 base pairs from the ends of either the forward or reverse strand consensus sequence alignments (alignment span excludes soft-clipped bases). Although this only negligibly alters call burdens (FIG. 8h), it removes rare alignment artifacts.

### Filter Based on Location Near Germline Reference Indels

Regions near germline indels are prone to alignment artifacts that can lead to false positive calls. This filter removes SBSs located less than twice the length of the indel or less than 15 base pairs of the indel, using indels called in any of the germline reference data variant calls of the individual (i.e. both GATK and DeepVariant indel calls when using Illumina germline reference data, and only DeepVariant indel calls when using PacBio germline reference data). For GATK indel calls, only indels with read depth≥5, QUAL≥10, genotype quality ≥5, and variant allele fraction≥0.2 were used in this filtering. For DeepVariant indel calls, only indels with read depth≥3, QUAL≥3, genotype quality ≥3, and variant allele fraction≥0.1 were used in this filtering.

### Filter Based on Location Near Consensus Sequence Indels

Regions near consensus sequence indels are prone to alignment artifacts that can lead to false positive calls. This filter removes SBSs located less than twice the length of a consensus sequence indel or less than 15 base pairs of a consensus sequence indel. For dsDNA SBSs, the SBS must pass this filter on both forward and reverse consensus strands. For ssDNA SBSs, this filter applies only to the strand containing the SBS.

### Filters Based on Germline Reference Read Depth and Variant Allele Fraction

1) Filter out SBSs in locations where the germline reference data has <15 total reads, since these low-coverage germline reference regions will be prone to false-negative germline variant calls that would then lead to false-positive somatic variant calls.
2) Filter out SBSs that were detected with variant allele fraction>0.05 or read depth>3 in the germline reference data to remove variants that were not called by the prior germline variant callers (due to low variant allele fraction or due to different local haplotype assembly in GATK/DeepVariant that calls variants in a different nearby location than the bwa alignment of the consensus molecule sequence). This filter is less stringent than a recent somatic mutation analysis method¹⁷, but may still remove a small number of very early developmental mosaic variants shared between the sample tissue and its germline reference.
Note: The above two filters use the samtools mpileup command to determine total read depth and variant allele fraction, using the parameters ‘-I-A-B-Q 11-ff 1024-d 10000-a “INFO/AD”’ for Illumina germline reference data and the parameters ‘-I-B-Q5-ff 2048-max-BQ 50-F0.1-025-e1-delta-BQ 10-M399999-d 10000-a “INFO/AD”’ for PacBio germline reference data.

### Filters Based on Fraction of Subreads (Passes) Detecting the Mutation and Fraction of Subreads Overlapping the Mutation

We filter out SBSs that were detected in <50% (for dsDNA mutation analysis) and <60% (for ssDNA mismatch and damage analysis) of the subreads belonging to the consensus sequence of the DNA molecule that detected the SBS. For dsDNA SBSs, this filter is applied to forward and reverse subreads separately, and the SBS must pass both. For ssDNA SBSs, this filter is applied only to subreads for the same strand in which the SBS was detected.

Note: This filter directly examines the subreads of each consensus sequence read (i.e. the subreads of each DNA molecule), in order to remove any false positive SBSs that are not well supported by the subreads. In order to apply these filters, the subreads of all the DNA molecules containing SBSs are extracted from the raw subreads BAM file using the zmwfilter tool (Pacific Biosciences) and aligned to the CHM13 v1.0 genome with pbmm2 with the parameters ‘-preset SUBREAD-sort’. The bcftools mpileup command is then used with parameters ‘-I-A-B-Q 0-d 10000-a “INFO/AD”’ to calculate the variant allele fraction of SBSs among subreads (excluding subreads with the supplementary alignment SAM flag).

In DNA molecules, when a large fraction, but not all, subreads are soft-clipped, false positive mutations can rarely occur in the small fraction of remaining subreads aligned to the soft-clipped region. We therefore also filter out SBSs for which the fraction of subreads overlapping the mutation (regardless of whether they contain the mutation) out of the total subreads aligned to the genome is <50%, taking into account for both the numerator and denominator terms only subreads from the same strand and molecule in which the mutation was found. This filter is applied separately to each strand in which the nucleotide change was found (i.e. only the mismatch-containing strand for ssDNA mutations, and to both strands for dsDNA mutations so that a dsDNA mutation must pass this filter in both strands).

### HiDEF-Seq Calculation of Mutation Burdens

Following application of all of the above filters, DNA molecules are further filtered to keep only DNA molecules that have a maximum of 1 dsDNA SBS for dsDNA mutation burden calculations, and a maximum of 1 ssDNA SBS per strand for ssDNA call burden calculations. This removes a small number of remaining DNA molecules that contain multiple somatic SBS calls that upon manual inspection are due to residual regions of the genome prone to false positives.

The raw dsDNA mutation burden of a sample is then calculated as the [#of dsDNA SBSs]/[#of interrogated dsDNA base pairs], and the raw ssDNA call burden is calculated as the [#of forward strand SBSs+#of reverse strand SBSs]/[#of interrogated forward strand read bases+#of interrogated reverse strand read bases]. Note that we subsequently use the term ‘interrogated bases’ for simplicity, even though for dsDNA mutation analysis it refers to interrogated base pairs. The number of interrogated bases takes into account all of the relevant filters that were applied, both filters that entirely remove DNA molecules and filters that remove only portions of DNA molecules. Specifically, the number of interrogated bases is the total number of bases of DNA molecules that passed all the filters that remove full DNA molecules (i.e., ‘Filters based on DNA molecule sequencing and alignment metrics’ and ‘Filters based on genomic regions—section A’), minus the bases of those remaining DNA molecules removed by the filters that only remove portions of DNA molecules: a) ‘Filters based on genomic regions—section B’, b) ‘Base quality filter’, c) ‘Filter based on location within the read’, d) Filter based on location near germline reference indels, e) ‘Filter based on location near consensus sequence indels’, and, f) ‘Filter based on germline reference read depth—minimum germline reference total read coverage’.

We then calculate call burdens after correcting for the trinucleotide distribution of the full genome (specifically, the CHM13 v1.0 sequences of chromosomes being analyzed; i.e., chromosomes 1-22 and X for nuclear genome analysis, and the mitochondrial sequence for mitochondrial genome analysis) relative to the trinucleotide distribution of interrogated bases in sequencing reads. This correction for ‘trinucleotide context opportunities’ is necessary because sequencing reads may have a different distribution of trinucleotides due to restriction enzyme fragmentation, and this may affect mutation burden estimates¹⁷. Specifically, we first calculate the distribution of trinucleotides (fraction of each trinucleotide out of all trinucleotides) across the genome. We then calculate the distribution of trinucleotides across interrogated bases of sequencing reads in that sample (i.e. read bases remaining after the filtering steps). Next, for each trinucleotide, we calculate the ratio of its fractional distribution in the full genome to its fractional distribution in the interrogated bases. Finally, the raw count of calls for each trinucleotide context is multiplied by its corresponding genome/interrogated bases trinucleotide ratio. Note that for ssDNA SBSs, trinucleotide context corrections are performed using all 64 possible trinucleotides and using strand-specific trinucleotide sequences of calls, interrogated bases, and the genome-specifically, for calls in strands aligning to the forward strand of the reference genome, the reverse complements of the call, interrogated read sequences, and genome are used for trinucleotide context corrections, because the sequence data produced by the sequencer has the directionality of the sequencer-synthesized strand rather than the DNA molecule's template strand. For dsDNA SBSs, trinucleotide context corrections are performed using all possible 32 trinucleotide contexts where the middle base is a pyrimidine.

We similarly estimated the number of calls per cell corrected for the trinucleotide distribution of the full genome relative to the trinucleotide distribution of interrogated bases. This calculation was performed as in the calculation of mutation burden corrected for the trinucleotide context of the genome, except that in the calculations, trinucleotide counts rather than fractions were used for the genome and interrogated bases. This corrects for the number of bases in the haploid genome relative to the number of interrogated bases to provide a per-haploid genome burden estimate. This per-haploid genome burden estimate (calls per cell) is the estimate for germline (e.g., sperm) cells, and twice this number is the estimate for diploid somatic cells.

Next, dsDNA mutation counts are sensitivity-corrected by dividing by the sensitivity of detection of a set of high-quality, true-positive heterozygous germline (dsDNA) variants present in the final interrogated bases. This specifically accounts for single-molecule sensitivity loss due to the ‘Filters based on fraction of subreads (passes) detecting the mutation and fraction of subreads overlapping the mutation’ that are applied to mutations detected in the final interrogated bases (they are applied to each strand separately, and dsDNA mutations must pass the filters in both strands); all other filters remove DNA molecules and bases from the final set of interrogated bases used for mutation burden calculations and therefore do not require a sensitivity correction. ssDNA counts are corrected by dividing by the square root of the germline variant sensitivity, because the above dsDNA sensitivity estimate corrects for filters applied to both strands separately. To generate the true-positive set of heterozygous germline variants, we first extract all the autosomal SBSs detected in the final interrogated HiDEF-seq bases that were also called in the germline reference variant call sets of the individual with at least 50^{th} percentile QUAL score, genotype quality, and total read depth, as well as between 30^{th} to 70^{th} percentile variant allele frequency. We keep only variants that meet these criteria across every one of the variant call sets of the individual, and we keep only variants detected in gnomAD v3.1.2 with ‘PASS’ flag and population allele frequency>0.1%. If >10,000 variants are identified, a random subset of 10,000 is selected for the sensitivity calculation. We then extract subreads corresponding to the DNA molecules that detected these variants in the sample, realign them to the genome, and annotate the variants using the same process described in the ‘Filters based on fraction of subreads (passes) detecting the mutation and fraction of subreads overlapping the mutation’ step of the mutation filtering pipeline. Finally, we calculate sensitivity as the number of true-positive germline variants passing the same filtering thresholds used in the ‘Filters based on fraction of subreads (passes) detecting the mutation and fraction of subreads overlapping the mutation’ step of the mutation analysis pipeline, divided by the total number of true-positive germline variants.

Finally, the ssDNA and dsDNA burdens corrected for both trinucleotide context and sensitivity are then calculated as the sum of the trinucleotide context- and sensitivity-corrected mismatch or mutation counts, respectively, divided by the number of interrogated bases or base pairs, respectively. For all analyses and figures, unless otherwise specified, we use burden estimates corrected for both sensitivity and the full genome trinucleotide distribution.

Poisson 95% confidence intervals of corrected mutation burdens were calculated as the corrected mutation burdens×[Poisson 95% confidence interval of raw mutation counts, calculated by the poisson·test function in R]/[raw mutation counts]. Linear weighted regressions of mutation burdens versus age were performed with the ‘lm’ function in R (via the ggplot package), with weights equal to 1/[raw mutation counts].

### NanoSeq Data Processing

NanosSeq data was processed using the standard NanoSeq analysis pipeline (with the hs37d5 reference genome).

### Mutational Signature Analysis

Mutational signature analysis for dsDNA mutations was performed using the ‘sigfit’ package¹²², with input of raw mutation counts for each trinucleotide context, and the ‘opportunities’ parameter set to the ratio of the frequency of each trinucleotide context in interrogated bases of that sample versus the frequency of that trinucleotide context in the human reference genome. Also, the correction for trinucleotide context opportunities performed above for burden analyses uses CHM13 v1.0, but the correction for trinucleotide context opportunities performed here for mutational signature analyses and figures uses trinucleotide frequencies of the full GRCh37 genome (for both nuclear and mitochondrial genome analyses and figures) so that the obtained spectra and signatures can be compared to standard COSMIC signatures. The ‘plot_gof’ function was used to determine the optimal number of signatures to extract. Since COSMIC SBS1 was not well separated from other signatures during de novo extraction¹²³, we utilized the ‘fit_extract_signatures’ function to fit SBS1 while simultaneously extracting additional signatures de novo. De novo extracted signatures were compared to the COSMIC SBS v3.2 catalogue³³ to identify the most similar known signature by cosine similarity. To obtain more accurate estimates of signature exposures, the fitted COSMIC SBS signature and the extracted signatures were then re-fit back to the mutation counts, along with correction for trinucleotide context opportunities using the ‘fit_signatures’ function. SBS5 is a ubiquitous clock-like signature³³, and often de novo extraction produced more than one signature highly similar to SBS5, for example, both SBS5 and SBS3 (cosine similarity 0.79) or both SBS5 and SBS40 (cosine similarity 0.83) or both SBS3 and SBS40 (cosine similarity 0.88). In these cases, we either reduced the number of de novo extracted signatures so that only one of these similar signatures was extracted, or we instructed ‘fit_extract_signatures’ to fit both COSMIC SBS1 and COSMIC SBS5.

ssDNA signatures were extracted by taking advantage of sigfit's capability to analyze 192-trinucleotide context mutational spectra that distinguish transcribed versus untranscribed strands. Instead, we use this feature to distinguish central pyrimidine versus central purine contexts. We do this by arbitrarily setting central pyrimidine and central purine ssDNA calls to the transcribed and untranscribed strands, respectively (by setting the strand column to ‘-1’ for all calls that are input into the ‘build_catalogues’ function, without collapsing central pyrimidine and central purine contexts). We then extract ssDNA signatures as described above for dsDNA signatures, with correction for trinucleotide context opportunities. Cosine similarities of ssDNA and dsDNA signatures are calculated after projecting ssDNA signatures to 96-central pyrimidine contexts (i.e., by summing values of central pyrimidine contexts with their reverse complement central purine contexts).

### Replication Strand Asymmetry (Fork Polarity) Analysis

ENCODE replication timing (Repli-seq) data¹²⁴ (wavelet-smoothed signal) was obtained from the UCSC Genome Browser (hg19) for the lymphoblastoid cell lines GM12878, GM06990, GM12801, GM12812, and GM12813. We calculated the average of the Repli-seq signal (higher values indicate earlier replication) across these samples at each position, and then lifted over the data to CHM13 v1.0. For each mutation (or mismatch) position, we calculated fork polarity¹²⁵ as the slope of the Repli-seq data points spanning −5 to +5 kilobases using the ‘lm’ function in R. Positive and negative fork polarities indicate the genome non-reference (−) strand is synthesized more frequently in the leading and lagging strand directions, respectively. This was also performed for a set of 50 iterations of 1,000 randomly selected genomic positions with either the sequence or the reverse complement of the sequence corresponding to the trinucleotide context being analyzed (i.e. AGA or TCT for POLE samples). We then calculated the fork polarity quantile values at quantiles ranging from 0 to 1.0 in 0.1 increments, and then for each of these quantile bins (combining 0.4-0.5 and 0.5-0.6 quantile bins into one bin, as these span fork polarity 0) we counted the number of loci whose sequence is AGA in the genome non-reference (−) strand and the number of loci whose sequence is AGA in the genome reference (+) strand. Loci without annotated Repli-seq data were excluded. Next, for each genome strand, we calculated normalized mutation (or mismatch) counts by dividing the quantile bin mutation (or mismatch) counts by the total number of mutations (or mismatches) in that strand. For each of the 9 quantile bins, we then calculated the ‘strand ratio’ as the ratio of non-reference to reference strand normalized mutation (or mismatch) counts. We also calculated this strand ratio for positive and negative fork polarities (i.e. two bins rather than 9 quantile bins), since there were not enough ssDNA mismatches in individual quantile bins for analysis. Analyses were also repeated after excluding loci within genic regions using the LiftOff Genes V2 annotation of the UCSC Genome Browser.

### Kinetics Analysis

For each sample, consensus sequences for each strand were created with pbccs version 6.4.0 (Pacific Biosciences) with parameters: -by-strand-hifi-kinetics-min-rq 0.99-top-passes 0. ccs version 6.4.0 was used because with these parameters it outputs consensus kinetics values for each strand separately, which prior version of pbccs do not, including the version of pbccs (6.2.0) used for the main HiDEF-seq pipeline. We did not upgrade the main HiDEF-seq pipeline to pbccs 6.4.0 because all analysis parameters and filters were optimized for pbccs 6.2.0. Consensus sequence reads were then aligned to the CHM13 v1.0 reference genome with pbmm2 (Pacific Biosciences) with the parameters ‘-preset CCS-sort’.

Next, we extracted the list of ssDNA C>T sequence calls in the heat-treated blood DNA and sperm samples (sequenced by HiDEF-seq v2). Due to the very high number of ssDNA C>T calls in blood DNA samples that were heat-treated in water-only or Tris-only buffer, for these samples we selected a random subset of 800 calls. We then extracted from these samples and from 88 other HiDEF-seq samples all the consensus reads that overlapped the C>T call positions, from the strand synthesized by the sequencing polymerase opposite to the strand on which the event is present in the molecule. Since kinetics is affected by sequence context⁶⁷, this allows calculation of differences in kinetics between molecules without and without the event within the same sequence context. Next, we performed kinetic analyses for interpulse duration (IPD) and pulse width (PW). Kinetics values (IPD or PW) for each read were transformed into units of time (seconds) and normalized by the average kinetics values of all bases in the read to correct for baseline sequencing kinetics differences between molecules. For each C>T sequencing event, we extracted the kinetics values for all overlapping reads for ±30 base pairs flanking the event position relative to the reference genome coordinates using each read's CIGAR value to account for insertions or deletions in the read relative to the reference. Next, for each C>T sequencing event, we calculated the ratio of kinetics values for each base position by dividing the kinetics values (IPD or PW) of the molecule with the event by the weighted average kinetics values of molecules without the event (the latter weighted by each molecule's number of passes; i.e., ‘ec’ tag). Finally, for each flanking and mutant base position, we calculated the average and standard error of the mean of the kinetics value ratios across all C>T sequencing events of each sample or sample set of interest. The same kinetic analysis was performed for dsDNA C>T mutation calls (i.e., bona fide cytosine to thymine double-strand mutations) in non-heat treated blood DNA, 56 C and 72 C heat treated blood DNA, sperm, kidney, and liver samples (all sequenced by HiDEF-seq v2), for the strands synthesized by the sequencing polymerase opposite the strand containing the C>T mutation; this shows the kinetic profile of true C>T events, as a comparator for C>T events arising from cytosine damage. Note, the dsDNA C>T mutations used for this kinetics analysis were called with the same thresholds used for ssDNA C>T calls. Both these ssDNA and dsDNA analyses were additionally conducted after randomization of labels among molecules with and without the C>T sequencing event to confirm the kinetic signal was specific to molecules with the C>T sequencing event. The kinetic profile heatmap and clustering was performed with the ‘ComplexHeatmap’ R package.

### Standard PacBio HiFi Data and Comparison to HiDEF-Seq Data

Standard PacBio HiFi raw subreads data for comparisons to HiDEF-seq were obtained from the Human Pangenome Reference Consortium (HPRC) public data repository¹²⁶ (samples HG02080, HG03098, HG02055, HG03492, HG02109, HG01442, HG02145, HG02004, HG01496, HG02083). Circular consensus sequences were derived from raw subreads using the same ccs version and ccs parameters used for HiDEF-seq data (-by-strand, -min-rq 0.99 and -top-passes 0).

### Paternally-Phased De Novo Mutation Burdens for Comparison to HiDEF-Seq Sperm Data

Paternally-phased de novo mutation (DNM) burdens were calculated for each paternal age (in 1-year intervals) from data of a prior study of 2,976 trios²⁰, and using additional methodological details obtained from its associated study by Jonsson, et al¹²⁷. Paternally-phased DNM burdens were first calculated for each child as [total number of DNMs]×[fraction of phased DNMs across the full cohort that are paternally phased]×[Jonsson et al's correction factor of 1.009 that accounts for its false positive and negative rate]/[Jonsson et al's interrogated genome size of 2,682,890,000]20.127. We then compare the mutation burden of each sperm sample to the DNM burdens of children whose fathers' age at their birth is 1-year higher than the age at which the sperm sample was collected (to account for ˜ 9 months between father's age at conception and birth).

### Supplementary Information

### Supplementary Note 1. HiDEF-Seq with Larger DNA Fragments

Since the number of passes in HiDEF-seq exceeded our initial goals, we analyzed whether HiDEF-seq with larger fragments may achieve greater efficiency for double-strand DNA (dsDNA) mutation detection, albeit with reduced efficiency for single-strand DNA (ssDNA) events that require a higher number of passes. As described in the methods for HiDEF-seq utilizing Hpy166II for 1-4 kilobase (kb) libraries, we similarly performed an in silico computational screen and experimental screen to identify a blunt-cutting restriction enzyme that fragments the human genome to a larger size range of approximately 1-10 kb. Experimental screening of the top in silico candidates identified Pvull as producing the desired fragment size distribution. We prepared large fragment HiDEF-seq libraries with Pvull from two sperm samples (SPM-1002 and SPM-1020; Supplementary Table 1 and

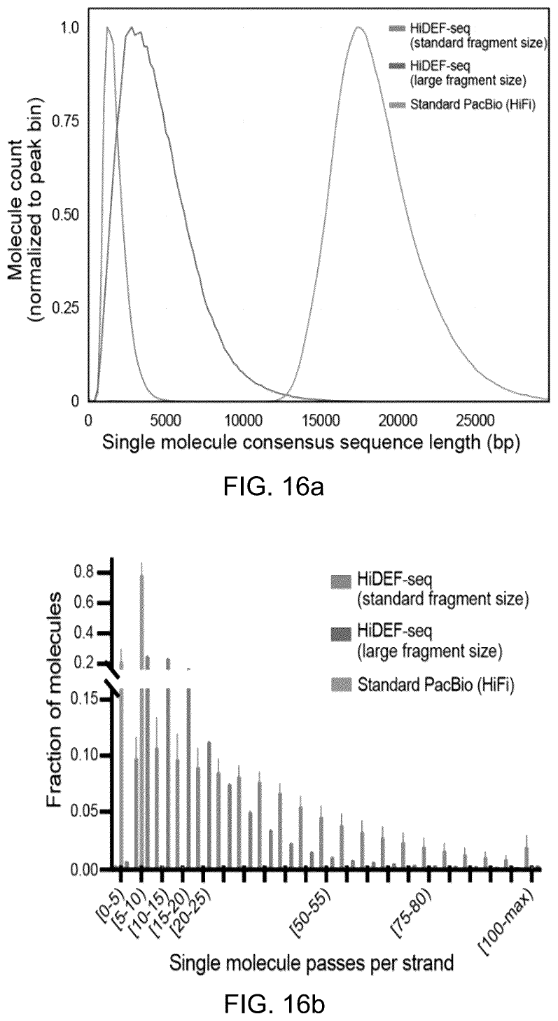

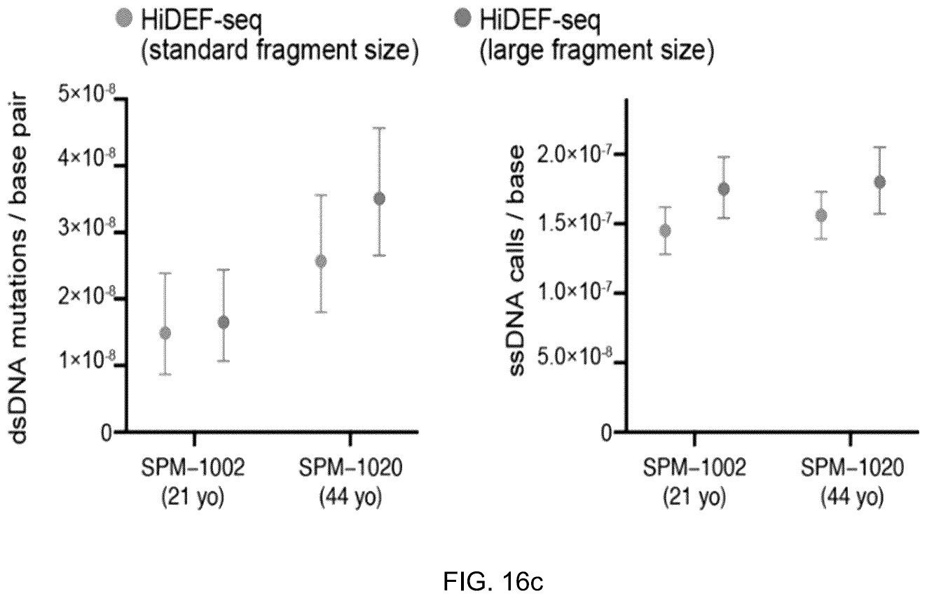

Methods). As expected, analysis of large fragment HiDEF-seq data showed a larger median fragment size compared to standard size HiDEF-seq (4.2 kb versus 1.7 kb, respectively) and a lower median number of passes (15.2 versus 32, respectively) (FIGS. 16a,b). Standard and large fragment size HiDEF-seq yielded an average of 2.6.109 and 3.9.109 interrogated dsDNA base pairs per sequencing run, respectively (the large fragment size run was in the 95% ile relative to standard fragment size runs), and 4.3.109 and 3.4.109 interrogated ssDNA bases, respectively. Therefore, HIDEF-seq with large fragment size improves efficiency for dsDNA interrogation, while reducing efficiency for ssDNA interrogation. dsDNA mutation and ssDNA call burdens were similar between large and standard fragment size HiDEF-seq for the same samples (FIG. 16c).

The reference listings in this disclosure is not an indication that any reference is material to patentability.

### REFERENCES

1 Moore, L. et al. The mutational landscape of human somatic and germline cells. Nature 597, 381-386 (2021).
2 Manders, F., van Boxtel, R. & Middelkamp, S. The Dynamics of Somatic Mutagenesis During Life in Humans. Frontiers in Aging 2 (2021).
3 Vijg, J. From DNA damage to mutations: All roads lead to aging. Ageing Research Reviews 68, 101316 (2021).
4 Seplyarskiy, V. B. & Sunyaev, S. The origin of human mutation in light of genomic data. Nature Reviews Genetics 22, 672-686 (2021).
5 Koh, G., Degasperi, A., Zou, X., Momen, S. & Nik-Zainal, S. Mutational signatures: emerging concepts, caveats and clinical applications. Nature Reviews Cancer 21, 619-637 (2021).
6 Lodato, M. A. et al. Aging and neurodegeneration are associated with increased mutations in single human neurons. Science 359, 555-559 (2018).
7 Evrony, G. D. et al. Single-neuron sequencing analysis of L1 retrotransposition and somatic mutation in the human brain. Cell 151, 483-496 (2012).
8 Evrony, G. D. et al. Cell lineage analysis in human brain using endogenous retroelements. Neuron 85, 49-59 (2015).
9 Blokzijl, F. et al. Tissue-specific mutation accumulation in human adult stem cells during life. Nature 538, 260-264 (2016).
10 Mitchell, E. et al. Clonal dynamics of haematopoiesis across the human lifespan. Nature 606, 343-350 (2022).
11 Lee-Six, H. et al. The landscape of somatic mutation in normal colorectal epithelial cells. Nature 574, 532-537 (2019).
12 Martincorena, I. et al. Somatic mutant clones colonize the human esophagus with age. Science 362, 911-917 (2018).
13 Abascal, F. et al. Somatic mutation landscapes at single-molecule resolution. Nature 593, 405-410 (2021).
14 Schmitt, M. W. et al. Detection of ultra-rare mutations by next-generation sequencing. Proceedings of the National Academy of Sciences 109, 14508 (2012).
15 Hoang, M. L. et al. Genome-wide quantification of rare somatic mutations in normal human tissues using massively parallel sequencing. 113, 9846-9851 (2016).
16 Bae, J. H. et al. CODEC enables ‘single duplex’ sequencing. bioRxiv, 2021.2006.2011.448110 (2021).
17 Abascal, F. et al. Somatic mutation landscapes at single-molecule resolution. Nature 593, 405-410 (2021).
18 Volkova, N. V. et al. Mutational signatures are jointly shaped by DNA damage and repair. Nature Communications 11, 2169 (2020).
19 Sloan, D. B., Broz, A. K., Sharbrough, J. & Wu, Z. Detecting Rare Mutations and DNA Damage with Sequencing-Based Methods. Trends in Biotechnology 36, 729-740 (2018).
20 Halldorsson, B. V. et al. Characterizing mutagenic effects of recombination through a sequence-level genetic map. Science 363, eaau1043 (2019).
21 Riva, L. et al. The mutational signature profile of known and suspected human carcinogens in mice. Nature Genetics 52, 1189-1197 (2020).
22 Freudenthal, Bret D., Beard, William A., Shock, David D. & Wilson, Samuel H. Observing a DNA Polymerase Choose Right from Wrong. Cell 154, 157-168 (2013).
23 Verderio, P. et al. External Quality Assurance programs for processing methods provide evidence on impact of preanalytical variables. New Biotechnology 72, 29-37 (2022).
24 Robinson, P. S. et al. Inherited MUTYH mutations cause elevated somatic mutation rates and distinctive mutational signatures in normal human cells. Nature Communications 13, 3949 (2022).
25 De Bont, R. & van Larebeke, N. Endogenous DNA damage in humans: a review of quantitative data. Mutagenesis 19, 169-185 (2004).
26 Wenger, A. M. et al. Accurate circular consensus long-read sequencing improves variant detection and assembly of a human genome. Nature Biotechnology 37, 1155-1162 (2019).
27 van Dijk, E. L., Jaszczyszyn, Y., Naquin, D. & Thermes, C. The Third Revolution in Sequencing Technology. Trends in Genetics 34, 666-681 (2018).
28 Matsuda, T., Matsuda, S. & Yamada, M. Mutation assay using single-molecule real-time (SMRT™) sequencing technology. Genes and Environment 37, 15 (2015).
29 Dohm, J. C., Peters, P., Stralis-Pavese, N. & Himmelbauer, H. Benchmarking of long-read correction methods. NAR Genomics and Bioinformatics 2, Iqaa037 (2020).
30 Vollger, M. R. et al. Segmental duplications and their variation in a complete human genome. bioRxiv, 2021.2005.2026.445678 (2021).
31 Nurk, S. et al. The complete sequence of a human genome. Science 376, 44-53 (2022).
32 Baid, G. et al. DeepConsensus: Gap-Aware Sequence Transformers for Sequence Correction. bioRxiv, 2021.2008.2031.458403 (2021).
33 Alexandrov, L. B. et al. The repertoire of mutational signatures in human cancer. Nature 578, 94-101 (2020).
34 Johnson, T. A. et al. Genomic features of renal cell carcinoma developed during end-stage renal disease and dialysis. Human Molecular Genetics 32, 290-303 (2022).
35 Xing, D., Tan, L., Chang, C. H., Li, H. & Xie, X. S. Accurate SNV detection in single cells by transposon-based whole-genome amplification of complementary strands. Proceedings of the National Academy of Sciences 118, e2013106118 (2021).
36 Robinson, P. S. et al. Increased somatic mutation burdens in normal human cells due to defective DNA polymerases. Nature Genetics 53, 1434-1442 (2021).
37 Zou, X. et al. A systematic CRISPR screen defines mutational mechanisms underpinning signatures caused by replication errors and endogenous DNA damage. Nature Cancer 2, 643-657 (2021).
38 Sanders, M. A. et al. Life without mismatch repair. bioRxiv, 2021.2004.2014.437578 (2021).
39 Yurchenko, A. A. et al. XPC deficiency increases risk of hematologic malignancies through mutator phenotype and characteristic mutational signature. Nature Communications 11, 5834 (2020).
40 Lujan, S. A., Williams, J. S. & Kunkel, T. A. DNA Polymerases Divide the Labor of Genome Replication. Trends in Cell Biology 26, 640-654 (2016).
41 Pursell, Z. F., Isoz, I., Lundstrom, E. B., Johansson, E. & Kunkel, T. A. Yeast DNA Polymerase Episolon Participates in Leading-Strand DNA Replication. Science 317, 127-130 (2007).
42 Lujan, S. A. et al. Heterogeneous polymerase fidelity and mismatch repair bias genome variation and composition. Genome Research 24, 1751-1764 (2014).
43 Shinbrot, E. et al. Exonuclease mutations in DNA polymerase epsilon reveal replication strand specific mutation patterns and human origins of replication. Genome Research 24, 1740-1750 (2014).
44 Tomkova, M., Tomek, J., Kriaucionis, S. & Schuster-Böckler, B. Mutational signature distribution varies with DNA replication timing and strand asymmetry. Genome Biology 19, 129 (2018).
45 Haradhvala, Nicholas J. et al. Mutational Strand Asymmetries in Cancer Genomes Reveal Mechanisms of DNA Damage and Repair. Cell 164, 538-549 (2016).
46 Shinmura, K. et al. Defective repair capacity of variant proteins of the DNA glycosylase NTHL1 for 5-hydroxyuracil, an oxidation product of cytosine. Free Radical Biology and Medicine 131, 264-273 (2019).
47 Thiviyanathan, V. et al. Base-pairing properties of the oxidized cytosine derivative, 5-hydroxy uracil. Biochemical and Biophysical Research Communications 366, 752-757 (2008).
48 Wagner, J. R., Hu, C. C. & Ames, B. N. Endogenous oxidative damage of deoxycytidine in DNA. Proceedings of the National Academy of Sciences 89, 3380-3384 (1992).
49 Duncan, B. K. & Miller, J. H. Mutagenic deamination of cytosine residues in DNA. Nature 287, 560-561 (1980).
50 Kreutzer, D. A. & Essigmann, J. M. Oxidized, deaminated cytosines are a source of C→T transitions<i> in vivo</i>. Proceedings of the National Academy of Sciences 95, 3578-3582 (1998).
51 Dizdaroglu, M. Oxidatively induced DNA damage and its repair in cancer. Mutation Research/Reviews in Mutation Research 763, 212-245 (2015).
52 Sentürker, S. et al. Oxidative DNA base damage and antioxidant enzyme levels in childhood acute lymphoblastic leukemia. FEBS Letters 416, 286-290 (1997).
53 England, T. et al. The steady-state levels of oxidative DNA damage and of lipid peroxidation (F2-isoprostanes) are not correlated in healthy human subjects. Free Radical Research 32, 355-362 (2000).
54 Chen, G., Mosier, S., Gocke, C. D., Lin, M. T. & Eshleman, J. R. Cytosine Deamination Is a Major Cause of Baseline Noise in Next-Generation Sequencing. Molecular Diagnosis & Therapy 18, 587-593 (2014).
55 Tretyakova, N., Villalta, P. W. & Kotapati, S. Mass Spectrometry of Structurally Modified DNA. Chemical Reviews 113, 2395-2436 (2013).
56 Carrà, A. et al. Targeted High Resolution LC/MS3 Adductomics Method for the Characterization of Endogenous DNA Damage. Frontiers in Chemistry 7 (2019).
57 Grolleman, J. E. et al. Mutational Signature Analysis Reveals NTHL1 Deficiency to Cause a Multi-tumor Phenotype. Cancer Cell 35, 256-266.e255 (2019).
58 Drost, J. et al. Use of CRISPR-modified human stem cell organoids to study the origin of mutational signatures in cancer. Science 358, 234-238 (2017).
59 Krokan, H. E. & Bjørås, M. Base Excision Repair. Cold Spring Harbor Perspectives in Biology 5 (2013).
60 Prasad, A., Wallace, S. S. & Pederson, D. S. Initiation of Base Excision Repair of Oxidative Lesions in Nucleosomes by the Human, Bifunctional DNA Glycosylase NTH1. Molecular and Cellular Biology 27, 8442-8453 (2007).
61 de Vega, M. & Salas, M. A highly conserved Tyrosine residue of family B DNA polymerases contributes to dictate translesion synthesis past 8-oxo-7,8-dihydro-2′-deoxyguanosine. Nucleic Acids Research 35, 5096-5107 (2007).
62 Chen, C. et al. Single-cell whole-genome analyses by Linear Amplification via Transposon Insertion (LIANTI). Science 356, 189-194 (2017).
63 Aitken, R. J. et al. Potential importance of transition metals in the induction of DNA damage by sperm preparation media. Human Reproduction 29, 2136-2147 (2014).
64 Newman, H., Catt, S., Vining, B., Vollenhoven, B. & Horta, F. DNA repair and response to sperm DNA damage in oocytes and embryos, and the potential consequences in ART: a systematic review. Molecular Human Reproduction 28 (2021).
65 Stringer, J. M., Winship, A., Liew, S. H. & Hutt, K. The capacity of oocytes for DNA repair. Cellular and Molecular Life Sciences 75, 2777-2792 (2018).
66 Guo, Q. et al. The mutational signatures of formalin fixation on the human genome. bioRxiv, 2021.2003.2011.434918 (2021).
67 Clark, T. A., Spittle, K. E., Turner, S. W. & Korlach, J. Direct Detection and Sequencing of Damaged DNA Bases. Genome Integrity 2, 10 (2011).
68 Schadt, E. E. et al. Modeling kinetic rate variation in third generation DNA sequencing data to detect putative modifications to DNA bases. Genome Research 23, 129-141 (2013).
69 Flusberg, B. A. et al. Direct detection of DNA methylation during single-molecule, real-time sequencing. Nature Methods 7, 461-465 (2010).
70 Owczarzy, R., Moreira, B. G., You, Y., Behlke, M. A. & Walder, J. A. Predicting Stability of DNA Duplexes in Solutions Containing Magnesium and Monovalent Cations. Biochemistry 47, 5336-5353 (2008).
71 Lindahl, T. & Nyberg, B. Heat-induced deamination of cytosine residues in deoxyribonucleic acid. Biochemistry 13, 3405-3410 (1974).
72 Kamiya, H. et al. 8-Hydroxyadenine (7, 8-dihydro-8-Oxoadenine) induces misincorporation in in vitro DNA synthesis and mutations in NIH 3T3 cells. Nucleic Acids Research 23, 2893-2899 (1995).
73 Sanchez-Contreras, M. et al. A replication-linked mutational gradient drives somatic mutation accumulation and influences germline polymorphisms and genome composition in mitochondrial DNA. Nucleic Acids Research 49, 11103-11118 (2021).
74 Ju, Y. S. et al. Origins and functional consequences of somatic mitochondrial DNA mutations in human cancer. Elife 3, e02935 (2014).
75 Kauppila, J. H. K. & Stewart, J. B. Mitochondrial DNA: Radically free of free-radical driven mutations. Biochimica et Biophysica Acta (BBA)—Bioenergetics 1847, 1354-1361 (2015).
76 Anderson, A. P., Luo, X., Russell, W. & Yin, Y. W. Oxidative damage diminishes mitochondrial DNA polymerase replication fidelity. Nucleic Acids Research 48, 817-829 (2019).
77 Kennedy, S. R., Salk, J. J., Schmitt, M. W. & Loeb, L. A. Ultra-Sensitive Sequencing Reveals an Age-Related Increase in Somatic Mitochondrial Mutations That Are Inconsistent with Oxidative Damage. PLOS Genetics 9, e1003794 (2013).
78 Zheng, W., Khrapko, K., Coller, H. A., Thilly, W. G. & Copeland, W. C. Origins of human mitochondrial point mutations as DNA polymerase γ-mediated errors. Mutation Research/Fundamental and Molecular Mechanisms of Mutagenesis 599, 11-20 (2006).
79 Shigenaga, M. K., Hagen, T. M. & Ames, B. N. Oxidative damage and mitochondrial decay in aging. Proceedings of the National Academy of Sciences 91, 10771-10778 (1994).
80 Yuan, Y. et al. Comprehensive molecular characterization of mitochondrial genomes in human cancers. Nature Genetics 52, 342-352 (2020).
81 Sanchez-Contreras, M. et al. Multi-tissue landscape of somatic mtDNA mutations indicates tissue specific accumulation and removal in aging. bioRxiv, 2022.2008.2030.505884 (2022).
82 Brown, T. A., Cecconi, C., Tkachuk, A. N., Bustamante, C. & Clayton, D. A. Replication of mitochondrial DNA occurs by strand displacement with alternative light-strand origins, not via a strand-coupled mechanism. Genes & Development 19, 2466-2476 (2005).
83 Alseth, I., Dalhus, B. & Bjørås, M. Inosine in DNA and RNA. Current Opinion in Genetics & Development 26, 116-123 (2014).
84 Sanchez-Contreras, M. & Kennedy, S. R. The Complicated Nature of Somatic mtDNA Mutations in Aging. Frontiers in Aging 2 (2022).
85 Fontana, G. A. & Gahlon, H. L. Mechanisms of replication and repair in mitochondrial DNA deletion formation. Nucleic Acids Research 48, 11244-11258 (2020).
86 Agarwal, A., Gupta, S. & Sharma, R. in Andrological Evaluation of Male Infertility: A Laboratory Guide (eds Ashok Agarwal, Sajal Gupta, & Rakesh Sharma) 101-107 (Springer International Publishing, 2016).
87 Wu, H., de Gannes, M. K., Luchetti, G. & Pilsner, J. R. Rapid method for the isolation of mammalian sperm DNA. Bio Techniques 58, 293-300 (2015).
88 Jenkins, T. G., Liu, L., Aston, K. I. & Carrell, D. T. Pre-screening method for somatic cell contamination in human sperm epigenetic studies. Systems Biology in Reproductive Medicine 64, 146-155 (2018).
89 Nurk, S. et al. The complete sequence of a human genome. bioRxiv, 2021.2005.2026.445798 (2021).
90 Heng, L. Aligning sequence reads, clone sequences and assembly contigs with BWA-MEM. arXiv (2013).
91 Broad-Institute. in Broad Institute, GitHub repository (2019).
92 Van der Auwera, G. A. & O'Connor, B. D. Genomics in the Cloud: Using Docker, GATK, and WDL in Terra. (O'Reilly Media, 2020).
93 Poplin, R. et al. A universal SNP and small-indel variant caller using deep neural networks. Nature Biotechnology 36, 983-987 (2018).
94 Li, H. et al. The Sequence Alignment/Map format and SAMtools. Bioinformatics 25, 2078-2079 (2009).
95 Li, H. Minimap2: pairwise alignment for nucleotide sequences. Bioinformatics 34, 3094-3100 (2018).
96 R-Core-Team. R: A Language and Environment for Statistical Computing. (2021).
97 Martin, M., Hervé, P., Valerie, O. & Nathaniel, H. Rsamtools: Binary alignment (BAM), FASTA, variant call (BCF), and tabix. (2020).
98 Lawrence, M. et al. Software for Computing and Annotating Genomic Ranges. PLOS Computational Biology 9, e1003118 (2013).
99 Knaus, B. J. & Grünwald, N. J. vcfr: a package to manipulate and visualize variant call format data in R. Molecular Ecology Resources 17, 44-53 (2017).
100 Wickham, H. The Split-Apply-Combine Strategy for Data Analysis. Journal of Statistical Software 40, 1-29 (2011).
101 Jianfeng, L. configr: An Implementation of Parsing and Writing Configuration File. (2020).
102 qs: Quick Serialization of R Objects (2021).
103 Robinson, J. T. et al. Integrative genomics viewer. Nature Biotechnology 29, 24-26 (2011).
104 Blokzijl, F., Janssen, R., van Boxtel, R. & Cuppen, E. Mutational Patterns: comprehensive genome-wide analysis of mutational processes. Genome Medicine 10, 33 (2018).
105 Milton, S. & Wickham, H. magrittr: A Forward-Pipe Operator for R. (2020).
106 Wickham, H., Hester, J. & Bryan, J. readr: Read Rectangular Text Data. (2022).
107 Wickham, H., François, R., Henry, L. & Müller, K. dplyr: A Grammar of Data Manipulation. (2021).
Lee, S., Cook, D. & Lawrence, M. plyranges: a grammar of genomic data transformation. Genome Biology 20, 4 (2019).
109 Wickham, H. stringr: Simple, Consistent Wrappers for Common String Operations. (2019).
110 Lawrence, M., Gentleman, R. & Carey, V. rtracklayer: an R package for interfacing with genome browsers. Bioinformatics 25, 1841-1842 (2009).
111 Danecek, P. et al. Twelve years of SAMtools and BCFtools. GigaScience 10, giab008 (2021).
112 Kuhn, R. M., Haussler, D. & Kent, W. J. The UCSC genome browser and associated tools. Briefings in Bioinformatics 14, 144-161 (2013).
113 Zerbino, D. R., Johnson, N., Juettemann, T., Wilder, S. P. & Flicek, P. WiggleTools: parallel processing of large collections of genome-wide datasets for visualization and statistical analysis. Bioinformatics 30, 1008-1009 (2014).
114 Eddelbuettel, D. digest: Create Compact Hash Digests of R Objects. (2021).
115 Shen, W., Le, S., Li, Y. & Hu, F. SeqKit: A Cross-Platform and Ultrafast Toolkit for FASTA/Q File Manipulation. PLOS ONE 11, e0163962 (2016).
116 Kokot, M., Długosz, M. & Deorowicz, S. KMC 3: counting and manipulating k-mer statistics. Bioinformatics 33, 2759-2761 (2017).
117 Hunt, M. et al. REAPR: a universal tool for genome assembly evaluation. Genome Biology 14, R47 (2013).
118 SMALT.
119 RepeatMasker Open-4.0 (2015).
120 Karimzadeh, M., Ernst, C., Kundaje, A. & Hoffman, M. M. Umap and Bismap: quantifying genome and methylome mappability. Nucleic Acids Research 46, e120-e120 (2018).
121 Karczewski, K. J. et al. The mutational constraint spectrum quantified from variation in 141,456 humans. Nature 581, 434-443 (2020).
122 Gori, K. & Baez-Ortega, A. sigfit: flexible Bayesian inference of mutational signatures. bioRxiv, 372896 (2020).
123 Cagan, A. et al. Somatic mutation rates scale with lifespan across mammals. Nature (2022).
124 Hansen, R. S. et al. Sequencing newly replicated DNA reveals widespread plasticity in human replication timing. Proceedings of the National Academy of Sciences 107, 139-144 (2010).
125 Seplyarskiy, V. B. et al. APOBEC-induced mutations in human cancers are strongly enriched on the lagging DNA strand during replication. Genome Research 26, 174-182 (2016).
126 Liao, W. W. et al. A Draft Human Pangenome Reference. bioRxiv, 2022.2007.2009.499321 (2022).
127 Jónsson, H. et al. Parental influence on human germline de novo mutations in 1,548 trios from Iceland. Nature 549, 519 (2017).

SUPPLEMENTARY TABLE 1
Sample information and sequencing metrics
Columns A-I

| | | | | | | Post- | | |
|---|---|---|---|---|---|---|---|---|
| | | | | | Tissue/cell | mortem | | DNA |
| Subject | Age | | | | type for HiDEF- | interval | DNA extraction | Integrity |
| ID | (years) | Sex | Disease | Disease details | seq or Nanoseq | (hours) | method | Number |
| 1443 | 0.9 | Female | — | — | Kidney | 10 | Nucleobond | 7.3 |
| | | | | | | | HMW, Magattract | |
| | | | | | | | HMW, Qiaamp | |
| 1104 | 35.3 | Male | — | — | Kidney | 12 | Nucleobond | 6.9 |
| | | | | | | | HMW, Magattract | |
| | | | | | | | HMW, Qiaamp | |
| 5697 | 67.2 | Male | — | — | Kidney | 15 | Nucleobond | 6.3 |
| | | | | | | | HMW, Magattract | |
| | | | | | | | HMW, Qiaamp | |
| 5840 | 75.4 | Male | — | — | Kidney | 17 | Nucleobond | 6.6 |
| | | | | | | | HMW, Magattract | |
| | | | | | | | HMW, Qiaamp | |
| 1443 | 0.9 | Female | — | — | Kidney | 10 | Nucleobond | 7.3 |
| | | | | | | | HMW, Magattract | |
| | | | | | | | HMW, Qiaamp | |
| 1104 | 35.3 | Male | — | — | Kidney | 12 | Nucleobond | 6.9 |
| | | | | | | | HMW, Magattract | |
| | | | | | | | HMW, Qiaamp | |
| 5697 | 67.2 | Male | — | — | Kidney | 15 | Nucleobond | 6.3 |
| | | | | | | | HMW, Magattract | |
| | | | | | | | HMW, Qiaamp | |
| 5840 | 75.4 | Male | — | — | Kidney | 17 | Nucleobond | 6.6 |
| | | | | | | | HMW, Magattract | |
| | | | | | | | HMW, Qiaamp | |
| 1443 | 0.9 | Female | — | — | Kidney | 10 | Puregene | 7.4 |
| 1104 | 35.3 | Male | — | — | Kidney | 12 | Puregene | 8.6 |
| 5697 | 67.2 | Male | — | — | Kidney | 15 | Puregene | 7.1 |
| 5840 | 75.4 | Male | — | — | Kidney | 17 | Puregene | 7.0 |
| 1443 | 0.9 | Female | — | — | Liver | 10 | Nucleobond | 7.9 |
| | | | | | | | HMW, Magattract | |
| | | | | | | | HMW, Qiaamp | |
| 1104 | 35.3 | Male | — | — | Liver | 12 | Nucleobond | 6.8 |
| | | | | | | | HMW, Magattract | |
| | | | | | | | HMW, Qiaamp | |
| 5697 | 67.2 | Male | — | — | Liver | 15 | Nucleobond | 6.1 |
| | | | | | | | HMW, Magattract | |
| | | | | | | | HMW, Qiaamp | |
| 5840 | 75.4 | Male | — | — | Liver | 17 | Nucleobond | 6.7 |
| | | | | | | | HMW, Magattract | |
| | | | | | | | HMW, Qiaamp | |
| 1443 | 0.9 | Female | — | — | Liver | 10 | Nucleobond | 7.9 |
| | | | | | | | HMW, Magattract | |
| | | | | | | | HMW, Qiaamp | |
| 1104 | 35.3 | Male | — | — | Liver | 12 | Nucleobond | 6.8 |
| | | | | | | | HMW, Magattract | |
| | | | | | | | HMW, Qiaamp | |
| 5697 | 67.2 | Male | — | — | Liver | 15 | Nucleobond | 6.1 |
| | | | | | | | HMW, Magattract | |
| | | | | | | | HMW, Qiaamp | |
| 5840 | 75.4 | Male | — | — | Liver | 17 | Nucleobond | 6.7 |
| | | | | | | | HMW, Magattract | |
| | | | | | | | HMW, Qiaamp | |
| 5697 | 67.2 | Male | — | — | Liver | 15 | Magattract HMW | 6.1 |
| 5697 | 67.2 | Male | — | — | Liver | 15 | Magattract HMW | 6.1 |
| 5697 | 67.2 | Male | — | — | Liver | 15 | Puregene | 7.0 |
| 5697 | 67.2 | Male | — | — | Liver | 15 | Puregene | 7.0 |
| 5697 | 67.2 | Male | — | — | Liver | 15 | Puregene | 7.0 |
| 5697 | 67.2 | Male | — | — | Liver | 15 | Puregene | 7.0 |
| 5697 | 67.2 | Male | — | — | Liver | 15 | Puregene | 7.0 |
| 5697 | 67.2 | Male | — | — | Liver | 15 | Puregene | 7.0 |
| 5697 | 67.2 | Male | — | — | Liver | 15 | Puregene | 7.0 |
| 5697 | 67.2 | Male | — | — | Liver | 15 | Puregene | 7.0 |
| 5697 | 67.2 | Male | — | — | Liver | 15 | Puregene | 7.0 |
| 5697 | 67.2 | Male | — | — | Liver | 15 | Puregene | 7.0 |
| 5697 | 67.2 | Male | — | — | Liver | 15 | Puregene | 7.0 |
| 5697 | 67.2 | Male | — | — | Liver | 15 | Puregene | 7.0 |
| 5697 | 67.2 | Male | — | — | Liver | 15 | Puregene | 7.0 |
| 1301 | 27 | Male | — | — | Blood | N/A | MagAttract HMW | 8.9 |
| 1901 | 62.4 | Male | Focal | Received kidney | Blood | N/A | MagAttract HMW | 9.1 |
| | | | segmental | transplant; 4.5 years | | | | |
| | | | glomerulo- | immunosupression | | | | |
| | | | sclerosis | treatment with | | | | |
| | | | | mycophenolate, | | | | |
| | | | | tacrolimus, and | | | | |
| | | | | prednisone. | | | | |
| 5344 | 25 | Female | — | — | Neurons (frontal | 4 | Qiaamp | 8.3 |
| | | | | | cortex) | | | |
| 6371 | 90 | Female | — | — | Neurons (frontal | 6 | Qiaamp | 7.5 |
| | | | | | cortex) | | | |
| SPM-1020 | 43.6 | Male | — | — | Sperm | N/A | Custom (bead | 8.0 |
| | | | | | | | milling, Qiagen | |
| | | | | | | | RLT, TCEP, | |
| | | | | | | | Qiaamp) | |
| SPM-1004 | 38.8 | Male | — | — | Sperm | N/A | Custom (bead | 7.7 |
| | | | | | | | milling, Qiagen | |
| | | | | | | | RLT, TCEP, | |
| | | | | | | | Qiaamp) | |
| 1301 | 27 | Male | — | — | Blood | N/A | MagAttract HMW | 8.9 |
| 1301 | 27 | Male | — | — | Blood | N/A | MagAttract HMW | 8.9 |
| 57627 | N/A | Male | PMS2 | PMS2 c.1831_1832insA | Lymphoblastoid | N/A | MagAttract HMW | 8.5 |
| | | | mismatch | (homozygous; | cell line | | | |
| | | | repair | NM_000535.7). T-Cell | | | | |
| | | | deficiency | Lymphoma (7 yo), | | | | |
| | | | | glioblastoma (8 yo). | | | | |
| | | | | Blood collected during | | | | |
| | | | | chemotherapy treatment. | | | | |
| 60603 | N/A | Male | PMS2 | PMS2 p.L731* | Lymphoblastoid | N/A | MagAttract HMW | 8.6 |
| | | | mismatch | (homozygous; | cell line | | | |
| | | | repair | NM_000535.7). | | | | |
| | | | deficiency | Glioblastoma (3 yo). | | | | |
| | | | | Blood collected after | | | | |
| | | | | tumor resection and | | | | |
| | | | | radiation therapy. | | | | |
| GM12812 | N/A | Male | — | — | Lymphoblastoid | N/A | MagAttract HMW | 8.8 |
| | | | | | cell line | | | |
| 58801 | N/A | Male | MSH6 | MSH6 c.3516_3519del | Lymphoblastoid | N/A | MagAttract HMW | 8.6 |
| | | | mismatch | (homozygous; | cell line | | | |
| | | | repair | NM_000179.3). T-Cell | | | | |
| | | | deficiency | Lymphoma (3 yo), Wilms | | | | |
| | | | | Tumour (3 yo). Blood | | | | |
| | | | | collected during | | | | |
| | | | | chemotherapy treatment. | | | | |
| 55838 | N/A | Female | MSH2 | MSH2 p.Gly315Ilefs*29/ | Lymphoblastoid | N/A | MagAttract HMW | 9.1 |
| | | | mismatch | p.I356R (compound | cell line | | | |
| | | | repair | heterozygous; | | | | |
| | | | deficiency | NM_000251.3). | | | | |
| | | | | Glioblastoma (2 yo). | | | | |
| | | | | Blood collected before | | | | |
| | | | | chemotherapy treatment. | | | | |
| 57615 | N/A | Female | POLE | POLE p.P436R | Lymphoblastoid | N/A | MagAttract HMW | 8.1 |
| | | | polymerase | (heterozygous; | cell line | | | |
| | | | proofreading- | NP_006222.2). | | | | |
| | | | associated | Astrocytoma (17 yo) and | | | | |
| | | | polyposis | intestinal | | | | |
| | | | syndrome | adenocarcinoma (17 yo). | | | | |
| | | | | Unknown treatment | | | | |
| | | | | history. | | | | |
| 59637 | N/A | Male | POLE | POLE p.S297F | Lymphoblastoid | N/A | MagAttract HMW | 9.2 |
| | | | polymerase | (heterozygous; | cell line | | | |
| | | | proofreading- | NP_006222.2). Colon | | | | |
| | | | associated | adenocarcinoma (20 yo) | | | | |
| | | | polyposis | and glioblastoma (36 yo). | | | | |
| | | | syndrome | Blood collected during | | | | |
| | | | | temozolomide treatment. | | | | |
| SPM-1020 | 43.6 | Male | — | — | Sperm | N/A | Custom (bead | 7.8 |
| | | | | | | | milling, Qiagen | |
| | | | | | | | RLT, TCEP, | |
| | | | | | | | Qiaamp) | |
| SPM-1004 | 38.8 | Male | — | — | Sperm | N/A | Custom (bead | 7.5 |
| | | | | | | | milling, Qiagen | |
| | | | | | | | RLT, TCEP, | |
| | | | | | | | Qiaamp) | |
| SPM-1002 | 21.2 | Male | — | — | Sperm | N/A | Custom (bead | 8.3 |
| | | | | | | | milling, Qiagen | |
| | | | | | | | RLT, TCEP, | |
| | | | | | | | Qiaamp) | |
| SPM-1013 | 18.3 | Male | — | — | Sperm | N/A | Custom (bead | 8.1 |
| | | | | | | | milling, Qiagen | |
| | | | | | | | RLT, TCEP, | |
| | | | | | | | Qiaamp) | |
| SPM-1060 | 48.7 | Male | — | — | Sperm | N/A | Custom (bead | 8.4 |
| | | | | | | | milling, Qiagen | |
| | | | | | | | RLT, TCEP, | |
| | | | | | | | Qiaamp) | |
| 1105 | 15.7 | Male | — | — | Blood | N/A | MagAttract HMW | 9.1 |
| 1400 | 6.3 | Male | PMS2 | PMS2 p.R3*/p.V43del | Blood | N/A | MagAttract HMW | 8.7 |
| | | | mismatch | (compound | | | | |
| | | | repair | heterozygous; | | | | |
| | | | deficiency | NP_000526.2). | | | | |
| | | | | Glioblastoma (6 yo). | | | | |
| | | | | Blood collected before | | | | |
| | | | | treatment. | | | | |
| 1901 | 62.4 | Male | Focal | Received kidney | Blood | N/A | MagAttract HMW | 9.1 |
| | | | segmental | transplant; 4.5 years | | | | |
| | | | glomerulo- | immunosupression | | | | |
| | | | sclerosis | treatment with | | | | |
| | | | | mycophenolate, | | | | |
| | | | | tacrolimus, and | | | | |
| | | | | prednisone. | | | | |
| 1901 | 62.4 | Male | Focal | Received kidney | Blood | N/A | MagAttract HMW −> | 9.1 |
| | | | segmental | transplant; 4.5 years | | | on ice 6 hours | |
| | | | glomerulo- | immunosupression | | | (buffer: 50 mM | |
| | | | sclerosis | treatment with | | | KAcetate, 20 mM | |
| | | | | mycophenolate, | | | Tris-Acetate, 10 | |
| | | | | tacrolimus, and | | | mM MgAcetate, | |
| | | | | prednisone. | | | 100 μg/ml | |
| | | | | | | | albumin, pH 7.9) | |
| 1901 | 62.4 | Male | Focal | Received kidney | Blood | N/A | MagAttract HMW −> | 9.1 |
| | | | segmental | transplant; 4.5 years | | | 56 C. 3 hours/ | |
| | | | glomerulo- | immunosupression | | | ice 3 hours | |
| | | | sclerosis | treatment with | | | (buffer: 50 mM | |
| | | | | mycophenolate, | | | KAcetate, 20 mM | |
| | | | | tacrolimus, and | | | Tris-Acetate, 10 | |
| | | | | prednisone. | | | mM MgAcetate, | |
| | | | | | | | 100 μg/ml | |
| | | | | | | | albumin, pH 7.9) | |
| 1901 | 62.4 | Male | Focal | Received kidney | Blood | N/A | MagAttract HMW −> | 9.1 |
| | | | segmental | transplant; 4.5 years | | | 56 C. 6 hours | |
| | | | glomerulo- | immunosupression | | | (buffer: 50 mM | |
| | | | sclerosis | treatment with | | | KAcetate, 20 mM | |
| | | | | mycophenolate, | | | Tris-Acetate, 10 | |
| | | | | tacrolimus, and | | | mM MgAcetate, | |
| | | | | prednisone. | | | 100 μg/ml | |
| | | | | | | | albumin, pH 7.9) | |
| 1901 | 62.4 | Male | Focal | Received kidney | Blood | N/A | MagAttract HMW −> | 9.1 |
| | | | segmental | transplant; 4.5 years | | | 72 C. 3 hours/ | |
| | | | glomerulo- | immunosupression | | | ice 3 hours | |
| | | | sclerosis | treatment with | | | (buffer: 50 mM | |
| | | | | mycophenolate, | | | KAcetate, 20 mM | |
| | | | | tacrolimus, and | | | Tris-Acetate, 10 | |
| | | | | prednisone. | | | mM MgAcetate, | |
| | | | | | | | 100 μg/ml | |
| | | | | | | | albumin, pH 7.9) | |
| 1901 | 62.4 | Male | Focal | Received kidney | Blood | N/A | MagAttract HMW −> | 9.1 |
| | | | segmental | transplant; 4.5 years | | | 72 C. 6 hours | |
| | | | glomerulo- | immunosupression | | | (buffer: 50 mM | |
| | | | sclerosis | treatment with | | | KAcetate, 20 mM | |
| | | | | mycophenolate, | | | Tris-Acetate, 10 | |
| | | | | tacrolimus, and | | | mM MgAcetate, | |
| | | | | prednisone. | | | 100 μg/ml | |
| | | | | | | | albumin, pH 7.9) | |
| 1901 | 62.4 | Male | Focal | Received kidney | Blood | N/A | MagAttract HMW−> | 9.1 |
| | | | segmental | transplant; 4.5 years | | | on ice 6 hours | |
| | | | glomerulo- | immunosupression | | | (buffer: N/A; only | |
| | | | sclerosis | treatment with | | | water) | |
| | | | | mycophenolate, | | | | |
| | | | | tacrolimus, and | | | | |
| | | | | prednisone. | | | | |
| 1901 | 62.4 | Male | Focal | Received kidney | Blood | N/A | MagAttract HMW −> | 9.1 |
| | | | segmental | transplant; 4.5 years | | | 72 C. 6 hours | |
| | | | glomerulo- | immunosupression | | | (buffer: 10 mM | |
| | | | sclerosis | treatment with | | | Tris-HCl, 10 mM | |
| | | | | mycophenolate, | | | MgAcetate, pH | |
| | | | | tacrolimus, and | | | 8.0) | |
| | | | | prednisone. | | | | |
| 1901 | 62.4 | Male | Focal | Received kidney | Blood | N/A | MagAttract HMW −> | 9.1 |
| | | | segmental | transplant; 4.5 years | | | 72 C. 6 hours | |
| | | | glomerulo- | immunosupression | | | (buffer: 10 mM | |
| | | | sclerosis | treatment with | | | Tris-HCl, 10 mM | |
| | | | | mycophenolate, | | | MgCl2, pH 8.0) | |
| | | | | tacrolimus, and | | | | |
| | | | | prednisone. | | | | |
| 1901 | 62.4 | Male | Focal | Received kidney | Blood | N/A | MagAttract HMW −> | 9.1 |
| | | | segmental | transplant; 4.5 years | | | 72 C. 6 hours | |
| | | | glomerulo- | immunosupression | | | (buffer: 10 mM | |
| | | | sclerosis | treatment with | | | Tris-HCl, 50 mM | |
| | | | | mycophenolate, | | | KCl, pH 8.0) | |
| | | | | tacrolimus, and | | | | |
| | | | | prednisone. | | | | |
| 1901 | 62.4 | Male | Focal | Received kidney | Blood | N/A | MagAttract HMW −> | 9.1 |
| | | | segmental | transplant; 4.5 years | | | 72 C. 6 hours | |
| | | | glomerulo- | immunosupression | | | (buffer: 20 mM | |
| | | | sclerosis | treatment with | | | Tris-Acetate, 50 | |
| | | | | mycophenolate, | | | mM KAcetate, 10 | |
| | | | | tacrolimus, and | | | mM MgAcetate, | |
| | | | | prednisone. | | | 100 μg/ml | |
| | | | | | | | albumin, pH 7.9) | |
| 1901 | 62.4 | Male | Focal | Received kidney | Blood | N/A | MagAttract HMW −> | 9.1 |
| | | | segmental | transplant; 4.5 years | | | 72 C. 6 hours | |
| | | | glomerulo- | immunosupression | | | (buffer: 10 mM | |
| | | | sclerosis | treatment with | | | Tris-HCl, pH 8.0) | |
| | | | | mycophenolate, | | | | |
| | | | | tacrolimus, and | | | | |
| | | | | prednisone. | | | | |
| 1901 | 62.4 | Male | Focal | Received kidney | Blood | N/A | MagAttract HMW −> | 9.1 |
| | | | segmental | transplant; 4.5 years | | | 72 C. 6 hours | |
| | | | glomerulo- | immunosupression | | | (buffer: N/A; only | |
| | | | sclerosis | treatment with | | | water) | |
| | | | | mycophenolate, | | | | |
| | | | | tacrolimus, and | | | | |
| | | | | prednisone. | | | | |
| 5203 | 4.1 | Female | Developmental | — | Blood | N/A | MagAttract HMW | 9.0 |
| | | | delay (no | | | | | |
| | | | genetic | | | | | |
| | | | diagnosis) | | | | | |
| 6501 | 43.0 | Male | — | — | Blood | N/A | MagAttract HMW | 8.0 |
| 1409 | 18.1 | Male | — | — | Kidney | 6 | Nucleobond | 6.6 |
| | | | | | | | HMW, Magattract | |
| | | | | | | | HMW, Qiaamp | |
| 1324 | 13 | Male | PMS2 | PMS2 Exons 7-16 | Blood | N/A | MagAttract HMW | 9.0 |
| | | | mismatch | deletion/c.353 + 2T > C | | | | |
| | | | repair | (compound | | | | |
| | | | deficiency | heterozygous; | | | | |
| | | | | NM_000535.7). | | | | |
| | | | | Glioblastoma (9 yo). | | | | |
| | | | | Preceding focal radiation | | | | |
| | | | | and nivolumab treatment. | | | | |
| 1325 | 8 | Male | PMS2 | PMS2 compound | Blood | N/A | MagAttract HMW | 9.1 |
| | | | mismatch | heterozygous (variants | | | | |
| | | | repair | cannot be disclosed due | | | | |
| | | | deficiency | to consent). | | | | |
| | | | | Glioblastoma (6 yo). | | | | |
| | | | | Preceding nivolumab | | | | |
| | | | | treatment. | | | | |
| 63143 | 15 | Female | POLE | POLE p.M444K | Blood | N/A | MagAttract HMW | 9.1 |
| | | | polymerase | (heterozygous; | | | | |
| | | | proofreading- | NP_006222.2). Glioma | | | | |
| | | | associated | (10 yo), intestinal | | | | |
| | | | polyposis | adenocarcinoma (13 yo). | | | | |
| | | | syndrome | Preceding fluorouracil | | | | |
| | | | | and oxaliplatin treatment. | | | | |
| GM02036 | 11 yo at | Female | — | — | Primary | N/A | MagAttract HMW | 9.4 |
| | sampling | | | | fibroblasts | | | |
| | (Passage 14) | | | | | | | |
| GM03348 | 10 yo at | Male | — | — | Primary | N/A | MagAttract HMW | 9.4 |
| | sampling | | | | fibroblasts | | | |
| | (Passage 7) | | | | | | | |
| GM16381 | 14 yo at | Male | XPC | XPC p.S140fs*146/ | Primary | N/A | MagAttract HMW | 9.4 |
| | sampling | | Xeroderma | p.K522* (compound | fibroblasts | | | |
| | (Passage 3) | | Pigmentosum | heterozygous; | | | | |
| | | | | NP_004619.3) | | | | |
| 1104 | 35.3 | Male | — | — | Liver | 12 | Magattract HMW | 6.7 |
| 5697 | 67.2 | Male | — | — | Liver | 15 | Magattract HMW | 6.1 |
| 5840 | 75.4 | Male | — | — | Liver | 17 | Magattract HMW | 7.1 |
| 5344 | 25 | Female | — | — | Neurons (frontal | 4 | Qiaamp | 8.3 |
| | | | | | cortex) | | | |
| 6371 | 90 | Female | — | — | Neurons (frontal | 6 | Magattract HMW | 7.9 |
| | | | | | cortex) | | | |
| 63143 | N/A | Female | POLE | POLE p.M444K | Lymphoblastoid | N/A | MagAttract HMW | 9.0 |
| | | | polymerase | (heterozygous; | cell line | | | |
| | | | proofreading- | NP_006222.2). Glioma | | | | |
| | | | associated | (10 yo), intestinal | | | | |
| | | | polyposis | adenocarcinoma (13 yo). | | | | |
| | | | syndrome | Preceding fluorouracil | | | | |
| | | | | and oxaliplatin treatment. | | | | |
| GM01629 | 10 yo at | Female | ERCC6 | ERCC6 p.R670W/ | Primary | N/A | MagAttract HMW | 9.5 |
| | sampling | | Cockayne | p.Y1179fs*1200 | fibroblasts | | | |
| | (Passage 9) | | syndrome | (compound | | | | |
| | | | | heterozygous; | | | | |
| | | | | NP_000115.1) | | | | |
| GM28257 | 1 yo at | Male | ERCC8 | ERCC8 p.E13*/ | Primary | N/A | MagAttract HMW | 9.5 |
| | sampling | | Cockayne | c.173 + 1G > A (compound | fibroblasts | | | |
| | (Passage 4) | | syndrome | heterozygous; | | | | |
| | | | | NM_000082.4) | | | | |
| CC-346-253 | 43 | Male | MUTYH | MUTYH p.Y179C/ | Blood | N/A | MagAttract HMW | 6.7 |
| | | | polyposis | p.G396D (compound | | | | |
| | | | | heterozygous; | | | | |
| | | | | NP_001121897.1). No | | | | |
| | | | | cancer diagnosis. | | | | |
| CC-388-290 | 51 | Female | MUTYH | MUTYH p.G396D | Blood | N/A | MagAttract HMW | 8.9 |
| | | | polyposis | (homozygous; | | | | |
| | | | | NP_001121897.1). No | | | | |
| | | | | cancer diagnosis. | | | | |
| CC-713-555 | 29 | Male | MUTYH | MUTYH p.Y179C/ | Blood | N/A | MagAttract HMW | 9.1 |
| | | | polyposis | p.G396D (compound | | | | |
| | | | | heterozygous; | | | | |
| | | | | NP_001121897.1). No | | | | |
| | | | | cancer diagnosis. | | | | |
| 1901 | 62.4 | Male | Focal | Received kidney | Blood | N/A | Puregene | 9.1 |
| | | | segmental | transplant; 4.5 years | | | | |
| | | | glomerulo- | immunosupression | | | | |
| | | | sclerosis | treatment with | | | | |
| | | | | mycophenolate, | | | | |
| | | | | tacrolimus, and | | | | |
| | | | | prednisone. | | | | |
| 1901 | 62.4 | Male | Focal | Received kidney | Blood | N/A | Puregene −> 72 C. | 9.1 |
| | | | segmental | transplant; 4.5 years | | | 6 hours (buffer: | |
| | | | glomerulo- | immunosupression | | | 10 mM Tris-HCl, | |
| | | | sclerosis | treatment with | | | 10 mM MgCl2, | |
| | | | | mycophenolate, | | | pH 8.0) | |
| | | | | tacrolimus, and | | | | |
| | | | | prednisone. | | | | |
| 1901 | 62.4 | Male | Focal | Received kidney | Blood | N/A | MagAttract HMW −> | 9.1 |
| | | | segmental | transplant; 4.5 years | | | Custom (bead | |
| | | | glomerulo- | immunosupression | | | milling, Qiagen | |
| | | | sclerosis | treatment with | | | RLT, TCEP, | |
| | | | | mycophenolate, | | | Qiaamp) | |
| | | | | tacrolimus, and | | | | |
| | | | | prednisone. | | | | |
| D1 | 40.4 | Male | — | — | Sperm | N/A | Custom (bead | 7.8 |
| | | | | | | | milling, Qiagen | |
| | | | | | | | RLT, TCEP, | |
| | | | | | | | Qiaamp) | |
| D1 | 40.4 | Male | — | — | Sperm | N/A | Custom (bead | 7.6 |
| | | | | | | | milling, Qiagen | |
| | | | | | | | RLT, TCEP, | |
| | | | | | | | Qiaamp) | |
| D2 | 49.6 | Male | — | — | Sperm | N/A | Custom (bead | 6.5 |
| | | | | | | | milling, Qiagen | |
| | | | | | | | RLT, TCEP, | |
| | | | | | | | Qiaamp) | |
| D2 | 49.6 | Male | — | — | Sperm | N/A | Custom (bead | 7.6 |
| | | | | | | | milling, Qiagen | |
| | | | | | | | RLT, TCEP, | |
| | | | | | | | Qiaamp) | |
| SPM-1020 | 43.6 | Male | — | — | Sperm | N/A | Custom (bead | 8.1 |
| | | | | | | | milling, Qiagen | |
| | | | | | | | RLT, TCEP, | |
| | | | | | | | Qiaamp) | |
| SPM-1002 | 21.2 | Male | — | — | Sperm | N/A | Custom (bead | 8.3 |
| | | | | | | | milling, Qiagen | |
| | | | | | | | RLT, TCEP, | |
| | | | | | | | Qiaamp) | |
| SPM-1002 | 21.2 | Male | — | — | Sperm | N/A | Custom (bead | 8.3 |
| | | | | | | | milling, Qiagen | |
| | | | | | | | RLT, TCEP, | |
| | | | | | | | Qiaamp) | |
| SPM-1004 | 38.8 | Male | — | — | Sperm | N/A | Custom (bead | 7.7 |
| | | | | | | | milling, Qiagen | |
| | | | | | | | RLT, TCEP, | |
| | | | | | | | Qiaamp) | |
| SPM-1020 | 43.6 | Male | — | — | Sperm | N/A | Custom (bead | 7.8 |
| | | | | | | | milling, Qiagen | |
| | | | | | | | RLT, TCEP, | |
| | | | | | | | Qiaamp) | |
| 1443 | 0.9 | Female | — | — | Kidney | 10 | Nucleobond | 7.3 |
| | | | | | | | HMW, Magattract | |
| | | | | | | | HMW, Qiaamp | |
| SPM-1013 | 18.3 | Male | — | — | Sperm | N/A | Custom (bead | 8.1 |
| | | | | | | | milling, Qiagen | |
| | | | | | | | RLT, TCEP, | |
| | | | | | | | Qiaamp) | |
| SPM-1060 | 48.7 | Male | — | — | Sperm | N/A | Custom (bead | 8.4 |
| | | | | | | | milling, Qiagen | |
| | | | | | | | RLT, TCEP, | |
| | | | | | | | Qiaamp) | |
| 1105 | 15.7 | Male | — | — | Blood | N/A | MagAttract HMW | 9.1 |
| 6501 | 43.0 | Male | — | — | Blood | N/A | MagAttract HMW | 8.0 |
| 63143 | 15 | Female | POLE | POLE p.M444K | Blood | N/A | MagAttract HMW | 9.1 |
| | | | polymerase | (heterozygous; | | | | |
| | | | proofreading- | NP_006222.2). Glioma | | | | |
| | | | associated | (10 yo), intestinal | | | | |
| | | | polyposis | adenocarcinoma (13 yo). | | | | |
| | | | syndrome | Preceding fluorouracil | | | | |
| | | | | and oxaliplatin treatment. | | | | |

Columns J-S

| | Nanoseq | | | | | | | | |
|---|---|---|---|---|---|---|---|---|---|
| | | | | | | | | Number of | |
| | | | | Number of | Median | Number of | | dsDNA | |
| | | | | DNA | molecule | ssDNA | | consensus | |
| | | | | molecules | size after | consensus | | base pairs | |
| | | | Median | after | completing | bases after | Number of | after | Number of |
| | | Number of | insert | completing | all primary | completing | interrogated | completing | interrogated |
| | | dsDNA | size | all primary | data | all primary | ssDNA | all primary | dsDNA |
| Library | Sequencing | interrogated | (base | data | processing | data | consensus | data | consensus |
| preparation | depth (Gb) | base pairs | pairs) | processing | (base pairs) | processing | bases | processing | base pairs |
| HiDEF-seq v1 | — | — | — | 2.72E+05 | 1679 | 9.90E+08 | 4.91E+08 | 4.95E+08 | 2.90E+08 |
| HiDEF-seq v1 | — | — | — | 2.51E+05 | 1652 | 8.96E+08 | 4.54E+08 | 4.48E+08 | 2.67E+08 |
| HiDEF-seq v1 | — | — | — | 2.55E+05 | 1650 | 8.97E+08 | 4.52E+08 | 4.49E+08 | 2.66E+08 |
| HiDEF-seq v1 | — | — | — | 2.53E+05 | 1656 | 9.05E+08 | 4.54E+08 | 4.53E+08 | 2.67E+08 |
| HiDEF-seq v2 | — | — | — | 8.44E+05 | 1786 | 3.14E+09 | 1.59E+09 | 1.57E+09 | 9.45E+08 |
| HiDEF-seq v2 | — | — | — | 8.91E+05 | 1778 | 3.30E+09 | 1.71E+09 | 1.65E+09 | 1.02E+09 |
| HiDEF-seq v2 | — | — | — | 7.93E+05 | 1786 | 2.93E+09 | 1.51E+09 | 1.46E+09 | 9.00E+08 |
| HiDEF-seq v2 | — | — | — | 9.06E+05 | 1772 | 3.33E+09 | 1.71E+09 | 1.66E+09 | 1.02E+09 |
| HiDEF-seq v2, | — | — | — | 6.47E+05 | 1751 | 2.39E+09 | 1.08E+09 | 1.20E+09 | 6.94E+08 |
| Klenow | | | | | | | | | |
| incubation with | | | | | | | | | |
| ddBTP/without | | | | | | | | | |
| dATP, with blunt | | | | | | | | | |
| adapters | | | | | | | | | |
| HiDEF-seq v2, | — | — | — | 9.32E+05 | 1771 | 3.50E+09 | 1.59E+09 | 1.75E+09 | 1.02E+09 |
| Klenow | | | | | | | | | |
| incubation with | | | | | | | | | |
| ddBTP/without | | | | | | | | | |
| dATP, with blunt | | | | | | | | | |
| adapters | | | | | | | | | |
| HiDEF-seq v2, | — | — | — | 3.27E+05 | 1712 | 1.18E+09 | 5.45E+08 | 5.92E+08 | 3.47E+08 |
| Klenow | | | | | | | | | |
| incubation with | | | | | | | | | |
| ddBTP/without | | | | | | | | | |
| dATP, with blunt | | | | | | | | | |
| adapters | | | | | | | | | |
| HiDEF-seq v2, | — | — | — | 9.13E+05 | 1727 | 3.35E+09 | 1.53E+09 | 1.68E+09 | 9.76E+08 |
| Klenow | | | | | | | | | |
| incubation with | | | | | | | | | |
| ddBTP/without | | | | | | | | | |
| dATP, with blunt | | | | | | | | | |
| adapters | | | | | | | | | |
| HiDEF-seq v1 | — | — | — | 6.60E+05 | 1922 | 2.67E+09 | 1.12E+09 | 1.34E+09 | 7.18E+08 |
| HiDEF-seq v1 | — | — | — | 5.49E+05 | 1832 | 2.12E+09 | 9.24E+08 | 1.06E+09 | 5.88E+08 |
| HiDEF-seq v1 | — | — | — | 6.00E+05 | 1913 | 2.37E+09 | 1.03E+09 | 1.19E+09 | 6.58E+08 |
| HiDEF-seq v1 | — | — | — | 5.60E+05 | 1866 | 2.18E+09 | 9.53E+08 | 1.09E+09 | 6.08E+08 |
| HiDEF-seq v2 | — | — | — | 5.57E+05 | 1664 | 1.99E+09 | 1.08E+09 | 9.92E+08 | 6.16E+08 |
| HiDEF-seq v2 | — | — | — | 5.77E+05 | 1636 | 2.01E+09 | 1.13E+09 | 1.01E+09 | 6.42E+08 |
| HiDEF-seq v2 | — | — | — | 6.87E+05 | 1682 | 2.45E+09 | 1.37E+09 | 1.23E+09 | 7.83E+08 |
| HiDEF-seq v2 | — | — | — | 5.94E+05 | 1604 | 2.02E+09 | 1.15E+09 | 1.01E+09 | 6.53E+08 |
| HiDEF-seq v2 | — | — | — | 4.83E+05 | 1578 | 1.60E+09 | 8.77E+08 | 8.02E+08 | 4.96E+08 |
| HiDEF-seq | — | — | — | 5.68E+05 | 1633 | 1.94E+09 | 1.05E+09 | 9.69E+08 | 5.96E+08 |
| v2 + PNK | | | | | | | | | |
| HiDEF-seq v2 | — | — | — | 6.20E+05 | 1620 | 2.12E+09 | 1.16E+09 | 1.06E+09 | 6.58E+08 |
| HiDEF-seq | — | — | — | 6.77E+05 | 1620 | 2.32E+09 | 1.26E+09 | 1.16E+09 | 7.17E+08 |
| v2 + PNK | | | | | | | | | |
| HiDEF-seq v2 | — | — | — | 3.93E+05 | 1212 | 1.03E+09 | 6.40E+08 | 5.16E+08 | 3.51E+08 |
| with Bst large | | | | | | | | | |
| fragment (45 C., | | | | | | | | | |
| 30 mins) | | | | | | | | | |
| HiDEF-seq v2 | — | — | — | 3.58E+05 | 1218 | 9.44E+08 | 5.81E+08 | 4.72E+08 | 3.19E+08 |
| with Bst 2.0 | | | | | | | | | |
| (45 C., 30 mins) | | | | | | | | | |
| HiDEF-seq v2 | — | — | — | 3.49E+05 | 1189 | 9.01E+08 | 5.55E+08 | 4.51E+08 | 3.04E+08 |
| with Bst 3.0 | | | | | | | | | |
| (45 C., 30 mins) | | | | | | | | | |
| HiDEF-seq v2 | — | — | — | 3.53E+05 | 1183 | 9.06E+08 | 5.60E+08 | 4.53E+08 | 3.07E+08 |
| with Isopol SD + | | | | | | | | | |
| (37 C., 30 mins) | | | | | | | | | |
| HiDEF-seq v2 + | — | — | — | 3.91E+05 | 1196 | 1.02E+09 | 6.33E+08 | 5.08E+08 | 3.46E+08 |
| inorganic | | | | | | | | | |
| pyrophasphatase | | | | | | | | | |
| HiDEF-seq v2 | — | — | — | 3.94E+05 | 1188 | 9.92E+08 | 6.28E+08 | 4.96E+08 | 3.38E+08 |
| with Bst 3.0 | | | | | | | | | |
| (45 C., 150 mins) | | | | | | | | | |
| HiDEF-seq v2 | — | — | — | 5.81E+05 | 1195 | 1.48E+09 | 9.35E+08 | 7.41E+08 | 5.04E+08 |
| with Bst 3.0 | | | | | | | | | |
| (37 C., 210 mins) | | | | | | | | | |
| HiDEF-seq v2 | — | — | — | 6.38E+05 | 1182 | 1.60E+09 | 1.02E+09 | 8.01E+08 | 5.50E+08 |
| with Isopol SD + | | | | | | | | | |
| (37 C., 210 mins) | | | | | | | | | |
| HiDEF-seq v2, | — | — | — | 1.67E+06 | 1474 | 5.23E+09 | 2.85E+09 | 2.62E+09 | 1.63E+09 |
| without Klenow | | | | | | | | | |
| incubation, with | | | | | | | | | |
| blunt adapters | | | | | | | | | |
| HiDEF-seq v2, | — | — | — | 4.81E+05 | 1360 | 1.44E+09 | 8.32E+08 | 7.22E+08 | 4.71E+08 |
| Klenow | | | | | | | | | |
| incubation with | | | | | | | | | |
| ddBTP/without | | | | | | | | | |
| dATP, with blunt | | | | | | | | | |
| adapters | | | | | | | | | |
| HiDEF-seq v2, | — | — | — | 1.04E+06 | 1414 | 3.20E+09 | 1.79E+09 | 1.60E+09 | 1.02E+09 |
| Klenow | | | | | | | | | |
| incubation | | | | | | | | | |
| without ddBTP/ | | | | | | | | | |
| without dATP, | | | | | | | | | |
| with blunt | | | | | | | | | |
| adapters | | | | | | | | | |
| HiDEF-seq v1 | — | — | — | 1.70E+06 | 1759 | 6.43E+09 | 2.19E+09 | 3.22E+09 | 1.53E+09 |
| HiDEF-seq v1 | — | — | — | 1.58E+06 | 1721 | 5.86E+09 | 2.05E+09 | 2.93E+09 | 1.43E+09 |
| HiDEF-seq v1 | — | — | — | 1.36E+06 | 1817 | 5.34E+09 | 2.51E+09 | 2.67E+09 | 1.52E+09 |
| HiDEF-seq v1 | — | — | — | 1.21E+06 | 1577 | 4.15E+09 | 2.07E+09 | 2.08E+09 | 1.22E+09 |
| HiDEF-seq v1 | — | — | — | 1.81E+06 | 1455 | 5.69E+09 | 2.65E+09 | 2.85E+09 | 1.64E+09 |
| HiDEF-seq v1 | — | — | — | 1.72E+06 | 1502 | 5.57E+09 | 2.56E+09 | 2.79E+09 | 1.60E+09 |
| HiDEF-seq v1 | — | — | — | 1.05E+06 | 1711 | 3.85E+09 | 1.69E+09 | 1.93E+09 | 1.05E+09 |
| HiDEF-seq v2 | — | — | — | 9.24E+05 | 1687 | 3.35E+09 | 1.47E+09 | 1.68E+09 | 9.11E+08 |
| HiDEF-seq v2 | — | — | — | 9.58E+05 | 1582 | 3.26E+09 | 1.19E+09 | 1.63E+09 | 8.41E+08 |
| HiDEF-seq v2 | — | — | — | 1.01E+06 | 1587 | 3.44E+09 | 1.27E+09 | 1.72E+09 | 8.92E+08 |
| HiDEF-seq v2 | — | — | — | 2.10E+06 | 1797 | 8.10E+09 | 3.22E+09 | 4.05E+09 | 2.13E+09 |
| HiDEF-seq v2 | — | — | — | 6.47E+05 | 1688 | 2.35E+09 | 9.19E+08 | 1.17E+09 | 6.26E+08 |
| HiDEF-seq v2 | — | — | — | 7.39E+05 | 1708 | 2.72E+09 | 1.05E+09 | 1.36E+09 | 7.18E+08 |
| HiDEF-seq v2 | — | — | — | 1.77E+06 | 1810 | 6.88E+09 | 2.90E+09 | 3.44E+09 | 1.88E+09 |
| HiDEF-seq v2 | — | — | — | 1.80E+06 | 1820 | 7.04E+09 | 2.97E+09 | 3.52E+09 | 1.91E+09 |
| HiDEF-seq v2 | — | — | — | 1.58E+06 | 1720 | 5.80E+09 | 2.46E+09 | 2.90E+09 | 1.57E+09 |
| HiDEF-seq v2 | — | — | — | 3.31E+06 | 1780 | 1.26E+10 | 5.81E+09 | 6.30E+09 | 3.54E+09 |
| HiDEF-seq v2 | — | — | — | 1.13E+06 | 1768 | 4.33E+09 | 2.18E+09 | 2.16E+09 | 1.26E+09 |
| HiDEF-seq v2 | — | — | — | 1.14E+06 | 1680 | 4.16E+09 | 2.13E+09 | 2.08E+09 | 1.24E+09 |
| HiDEF-seq v2 | — | — | — | 1.27E+06 | 1677 | 4.63E+09 | 2.37E+09 | 2.31E+09 | 1.38E+09 |
| HiDEF-seq v2 | — | — | — | 9.67E+05 | 1476 | 3.15E+09 | 1.67E+09 | 1.57E+09 | 9.68E+08 |
| HiDEF-seq v2 | — | — | — | 8.61E+05 | 1483 | 2.82E+09 | 1.49E+09 | 1.41E+09 | 8.62E+08 |
| HiDEF-seq v2 | — | — | — | 8.72E+05 | 1490 | 2.87E+09 | 1.52E+09 | 1.43E+09 | 8.83E+08 |
| HiDEF-seq v2 | — | — | — | 5.66E+05 | 1748 | 2.14E+09 | 9.75E+08 | 1.07E+09 | 5.94E+08 |
| HiDEF-seq v2 | — | — | — | 5.68E+05 | 1746 | 2.15E+09 | 9.77E+08 | 1.08E+09 | 5.95E+08 |
| HiDEF-seq v2 | — | — | — | 5.33E+05 | 1731 | 2.00E+09 | 9.07E+08 | 9.99E+08 | 5.54E+08 |
| HiDEF-seq v2 | — | — | — | 5.87E+05 | 1718 | 2.18E+09 | 9.98E+08 | 1.09E+09 | 6.08E+08 |
| HiDEF-seq v2 | — | — | — | 5.16E+05 | 1690 | 1.88E+09 | 8.91E+08 | 9.41E+08 | 5.38E+08 |
| HiDEF-seq v2 | — | — | — | 3.54E+05 | 1722 | 1.33E+09 | 7.10E+08 | 6.67E+08 | 4.01E+08 |
| HiDEF-seq v2 | — | — | — | 3.43E+05 | 1681 | 1.24E+09 | 6.89E+08 | 6.21E+08 | 3.85E+08 |
| HiDEF-seq v2 | — | — | — | 3.32E+05 | 1712 | 1.24E+09 | 6.65E+08 | 6.18E+08 | 3.74E+08 |
| HiDEF-seq v2 | — | — | — | 3.12E+05 | 1694 | 1.14E+09 | 6.32E+08 | 5.68E+08 | 3.53E+08 |
| HiDEF-seq v2 | — | — | — | 3.68E+05 | 1683 | 1.34E+09 | 7.33E+08 | 6.71E+08 | 4.11E+08 |
| HiDEF-seq v2 | — | — | — | 1.23E+05 | 1377 | 3.57E+08 | 2.05E+08 | 1.78E+08 | 1.13E+08 |
| HiDEF-seq v2 | — | — | — | 4.75E+04 | 1355 | 1.36E+08 | 7.67E+07 | 6.79E+07 | 4.27E+07 |
| HiDEF-seq v2 | — | — | — | 6.96E+05 | 1516 | 2.28E+09 | 6.71E+08 | 1.14E+09 | 5.29E+08 |
| HiDEF-seq v2 | — | — | — | 6.23E+05 | 1522 | 2.04E+09 | 6.00E+08 | 1.02E+09 | 4.72E+08 |
| HiDEF-seq v2 | — | — | — | 6.71E+05 | 1494 | 2.14E+09 | 6.51E+08 | 1.07E+09 | 5.08E+08 |
| HiDEF-seq v2 | — | — | — | 8.84E+05 | 1734 | 3.23E+09 | 1.49E+09 | 1.62E+09 | 9.35E+08 |
| HiDEF-seq v2 | — | — | — | 9.28E+05 | 1719 | 3.37E+09 | 1.53E+09 | 1.68E+09 | 9.75E+08 |
| HiDEF-seq v2 | — | — | — | 8.60E+05 | 1700 | 3.08E+09 | 1.43E+09 | 1.54E+09 | 8.95E+08 |
| HiDEF-seq v2 | — | — | — | 7.30E+05 | 1813 | 2.85E+09 | 1.39E+09 | 1.43E+09 | 8.20E+08 |
| HiDEF-seq v2 | — | — | — | 7.99E+05 | 1812 | 3.12E+09 | 1.50E+09 | 1.56E+09 | 8.83E+08 |
| HiDEF-seq v2 | — | — | — | 7.80E+05 | 1838 | 3.08E+09 | 1.49E+09 | 1.54E+09 | 8.77E+08 |
| HiDEF-seq v2 | — | — | — | 5.01E+05 | 1618 | 1.74E+09 | 9.61E+08 | 8.69E+08 | 5.47E+08 |
| HiDEF-seq v2 | — | — | — | 5.45E+05 | 1633 | 1.90E+09 | 1.03E+09 | 9.51E+08 | 5.85E+08 |
| HiDEF-seq v2 | — | — | — | 4.02E+05 | 1532 | 1.31E+09 | 7.21E+08 | 6.54E+08 | 4.09E+08 |
| HiDEF-seq v2 | — | — | — | 1.67E+06 | 1896 | 6.84E+09 | 3.42E+09 | 3.42E+09 | 2.05E+09 |
| HiDEF-seq v2 | — | — | — | 8.36E+05 | 2006 | 3.61E+09 | 1.73E+09 | 1.81E+09 | 1.05E+09 |
| HiDEF-seq v2 | — | — | — | 8.17E+05 | 1891 | 3.34E+09 | 1.65E+09 | 1.67E+09 | 9.70E+08 |
| HiDEF-seq v2 | — | — | — | 8.67E+05 | 1854 | 3.49E+09 | 1.72E+09 | 1.74E+09 | 1.01E+09 |
| HiDEF-seq v2 | — | — | — | 1.01E+06 | 2052 | 4.41E+09 | 2.08E+09 | 2.20E+09 | 1.24E+09 |
| HiDEF-seq v2 | — | — | — | 1.06E+06 | 1697 | 3.84E+09 | 1.71E+09 | 1.92E+09 | 1.08E+09 |
| HiDEF-seq v2 | — | — | — | 1.17E+06 | 1699 | 4.26E+09 | 1.89E+09 | 2.13E+09 | 1.19E+09 |
| HiDEF-seq v2 | — | — | — | 1.04E+06 | 1691 | 3.74E+09 | 1.68E+09 | 1.87E+09 | 1.05E+09 |
| HiDEF-seq v2 | — | — | — | 1.06E+06 | 1592 | 3.60E+09 | 1.44E+09 | 1.80E+09 | 9.54E+08 |
| HiDEF-seq v2 | — | — | — | 4.31E+05 | 1664 | 1.58E+09 | 7.60E+08 | 7.89E+08 | 4.47E+08 |
| HiDEF-seq v2 | — | — | — | 4.29E+05 | 1600 | 1.50E+09 | 7.56E+08 | 7.52E+08 | 4.40E+08 |
| HiDEF-seq v2 | — | — | — | 3.63E+05 | 1594 | 1.27E+09 | 7.22E+08 | 6.34E+08 | 3.98E+08 |
| HiDEF-seq v2 | — | — | — | 5.13E+05 | 1648 | 1.85E+09 | 1.03E+09 | 9.25E+08 | 5.70E+08 |
| HiDEF-seq v2 | — | — | — | 5.10E+05 | 1610 | 1.78E+09 | 1.04E+09 | 8.89E+08 | 5.78E+08 |
| HiDEF-seq v2 | — | — | — | 5.94E+05 | 1547 | 2.01E+09 | 1.18E+09 | 1.01E+09 | 6.53E+08 |
| HiDEF-seq v2 | — | — | — | 1.09E+06 | 4106 | 9.89E+09 | 1.62E+09 | 4.94E+09 | 1.83E+09 |
| (Large fragment) | | | | | | | | | |
| HiDEF-seq v2 | — | — | — | 1.25E+06 | 4204 | 1.15E+10 | 1.81E+09 | 5.76E+09 | 2.09E+09 |
| (Large fragment) | | | | | | | | | |
| Nanoseq | 302.0 | 7.47E+09 | 404 | — | — | — | — | — | — |
| Nanoseq | 83.6 | 1.81E+09 | 312 | — | — | — | — | — | — |
| Nanoseq | 92.3 | 3.65E+09 | 410 | — | — | — | — | — | — |
| Nanoseq | 100.5 | 3.67E+09 | 399 | — | — | — | — | — | — |
| Nanoseq | 152.6 | 6.06E+09 | 421 | — | — | — | — | — | — |
| Nanoseq | 164 | 5.71E+09 | 429 | — | — | — | — | — | — |
| Nanoseq | 240.8 | 7.13E+09 | 437 | — | — | — | — | — | — |
| Nanoseq | 236.1 | 7.70E+09 | 411 | — | — | — | — | — | — |
| Nanoseq | 221.8 | 7.43E+09 | 414 | — | — | — | — | — | — |

SUPPLEMENTARY TABLE 2
Double-strand mutation and single-strand nucleotide change burdens
Supplementary Table 2 - Double-strand mutation single-strand nucleotide change burdens
Columns A-H

| | | | | Post- | | | |
|---|---|---|---|---|---|---|---|
| | | | Tissue/cell type | mortem | | DNA | |
| Subject | Age | | for HiDEF-seq or | interval | | Integrity | |
| ID | (years) | Sex | Nanoseq | (hours) | DNA extraction method | Number | Library preparation |
| 1443 | 0.9 | Female | Kidney | 10 | Nucleobond HMW, | 7.3 | HiDEF-seq v1 |
| | | | | | Magattract HMW, Qiaamp | | |
| 1104 | 35.3 | Male | Kidney | 12 | Nucleobond HMW, | 6.9 | HiDEF-seq v1 |
| | | | | | Magattract HMW, Qiaamp | | |
| 5697 | 67.2 | Male | Kidney | 15 | Nucleobond HMW, | 6.3 | HiDEF-seq v1 |
| | | | | | Magattract HMW, Qiaamp | | |
| 5840 | 75.4 | Male | Kidney | 17 | Nucleobond HMW, | 6.6 | HiDEF-seq v1 |
| | | | | | Magattract HMW, Qiaamp | | |
| 1443 | 0.9 | Female | Kidney | 10 | Nucleobond HMW, | 7.3 | HiDEF-seq v2 |
| | | | | | Magattract HMW, Qiaamp | | |
| 1104 | 35.3 | Male | Kidney | 12 | Nucleobond HMW, | 6.9 | HiDEF-seq v2 |
| | | | | | Magattract HMW, Qiaamp | | |
| 5697 | 67.2 | Male | Kidney | 15 | Nucleobond HMW, | 6.3 | HiDEF-seq v2 |
| | | | | | Magattract HMW, Qiaamp | | |
| 5840 | 75.4 | Male | Kidney | 17 | Nucleobond HMW, | 6.6 | HiDEF-seq v2 |
| | | | | | Magattract HMW, Qiaamp | | |
| 1443 | 0.9 | Female | Kidney | 10 | Puregene | 7.4 | HiDEF-seq v2, Klenow |
| | | | | | | | incubation with ddBTP/ |
| | | | | | | | without dATP, with blunt |
| | | | | | | | adapters |
| 1104 | 35.3 | Male | Kidney | 12 | Puregene | 8.6 | HiDEF-seq v2, Klenow |
| | | | | | | | incubation with ddBTP/ |
| | | | | | | | without dATP, with blunt |
| | | | | | | | adapters |
| 5697 | 67.2 | Male | Kidney | 15 | Puregene | 7.1 | HiDEF-seq v2, Klenow |
| | | | | | | | incubation with ddBTP/ |
| | | | | | | | without dATP, with blunt |
| | | | | | | | adapters |
| 5840 | 75.4 | Male | Kidney | 17 | Puregene | 7.0 | HiDEF-seq v2, Klenow |
| | | | | | | | incubation with ddBTP/ |
| | | | | | | | without dATP, with blunt |
| | | | | | | | adapters |
| 1443 | 0.9 | Female | Liver | 10 | Nucleobond HMW, | 7.9 | HiDEF-seq v1 |
| | | | | | Magattract HMW, Qiaamp | | |
| 1104 | 35.3 | Male | Liver | 12 | Nucleobond HMW, | 6.8 | HiDEF-seq v1 |
| | | | | | Magattract HMW, Qiaamp | | |
| 5697 | 67.2 | Male | Liver | 15 | Nucleobond HMW, | 6.1 | HiDEF-seq v1 |
| | | | | | Magattract HMW, Qiaamp | | |
| 5840 | 75.4 | Male | Liver | 17 | Nucleobond HMW, | 6.7 | HiDEF-seq v1 |
| | | | | | Magattract HMW, Qiaamp | | |
| 1443 | 0.9 | Female | Liver | 10 | Nucleobond HMW, | 7.9 | HiDEF-seq v2 |
| | | | | | Magattract HMW, Qiaamp | | |
| 1104 | 35.3 | Male | Liver | 12 | Nucleobond HMW, | 6.8 | HiDEF-seq v2 |
| | | | | | Magattract HMW, Qiaamp | | |
| 5697 | 67.2 | Male | Liver | 15 | Nucleobond HMW, | 6.1 | HiDEF-seq v2 |
| | | | | | Magattract HMW, Qiaamp | | |
| 5840 | 75.4 | Male | Liver | 17 | Nucleobond HMW, | 6.7 | HiDEF-seq v2 |
| | | | | | Magattract HMW, Qiaamp | | |
| 5697 | 67.2 | Male | Liver | 15 | Magattract HMW | 6.1 | HiDEF-seq v2 |
| 5697 | 67.2 | Male | Liver | 15 | Magattract HMW | 6.1 | HiDEF-seq v2 + PNK |
| 5697 | 67.2 | Male | Liver | 15 | Puregene | 7.0 | HiDEF-seq v2 |
| 5697 | 67.2 | Male | Liver | 15 | Puregene | 7.0 | HiDEF-seq v2 + PNK |
| 5697 | 67.2 | Male | Liver | 15 | Puregene | 7.0 | HiDEF-seq v2 with Bst |
| | | | | | | | large fragment (45 C., 30 |
| | | | | | | | mins) |
| 5697 | 67.2 | Male | Liver | 15 | Puregene | 7.0 | HiDEF-seq v2 with Bst 2.0 |
| | | | | | | | (45 C., 30 mins) |
| 5697 | 67.2 | Male | Liver | 15 | Puregene | 7.0 | HiDEF-seq v2 with Bst 3.0 |
| | | | | | | | (45 C., 30 mins) |
| 5697 | 67.2 | Male | Liver | 15 | Puregene | 7.0 | HiDEF-seq v2 with Isopol |
| | | | | | | | SD + (37 C., 30 mins) |
| 5697 | 67.2 | Male | Liver | 15 | Puregene | 7.0 | HiDEF-seq v2 + inorganic |
| | | | | | | | pyrophasphatase |
| 5697 | 67.2 | Male | Liver | 15 | Puregene | 7.0 | HiDEF-seq v2 with Bst 3.0 |
| | | | | | | | (45 C., 150 mins) |
| 5697 | 67.2 | Male | Liver | 15 | Puregene | 7.0 | HiDEF-seq v2 with Bst 3.0 |
| | | | | | | | (37 C., 210 mins) |
| 5697 | 67.2 | Male | Liver | 15 | Puregene | 7.0 | HiDEF-seq v2 with Isopol |
| | | | | | | | SD + (37 C., 210 mins) |
| 5697 | 67.2 | Male | Liver | 15 | Puregene | 7.0 | HiDEF-seq v2, without |
| | | | | | | | Klenow incubation, with |
| | | | | | | | blunt adapters |
| 5697 | 67.2 | Male | Liver | 15 | Puregene | 7.0 | HiDEF-seq v2, Klenow |
| | | | | | | | incubation with ddBTP/ |
| | | | | | | | without dATP, with blunt |
| | | | | | | | adapters |
| 5697 | 67.2 | Male | Liver | 15 | Puregene | 7.0 | HiDEF-seq v2, Klenow |
| | | | | | | | incubation without ddBTP/ |
| | | | | | | | without dATP, with blunt |
| | | | | | | | adapters |
| 1301 | 27 | Male | Blood | N/A | MagAttract HMW | 8.9 | HiDEF-seq v1 |
| 1901 | 62.4 | Male | Blood | N/A | MagAttract HMW | 9.1 | HiDEF-seq v1 |
| 5344 | 25 | Female | Neurons (frontal | 4 | Qiaamp | 8.3 | HiDEF-seq v1 |
| | | | cortex) | | | | |
| 6371 | 90 | Female | Neurons (frontal | 6 | Qiaamp | 7.5 | HiDEF-seq v1 |
| | | | cortex) | | | | |
| SPM-1020 | 43.6 | Male | Sperm | N/A | Custom (bead milling, | 8.0 | HiDEF-seq v1 |
| | | | | | Qiagen RLT, TCEP, | | |
| | | | | | Qiaamp) | | |
| SPM-1004 | 38.8 | Male | Sperm | N/A | Custom (bead milling, | 7.7 | HiDEF-seq v1 |
| | | | | | Qiagen RLT, TCEP, | | |
| | | | | | Qiaamp) | | |
| 1301 | 27 | Male | Blood | N/A | MagAttract HMW | 8.9 | HiDEF-seq v1 |
| 1301 | 27 | Male | Blood | N/A | MagAttract HMW | 8.9 | HiDEF-seq v2 |
| 57627 | N/A | Male | Lymphoblastoid | N/A | MagAttract HMW | 8.5 | HiDEF-seq v2 |
| | | | cell line | | | | |
| 60603 | N/A | Male | Lymphoblastoid | N/A | MagAttract HMW | 8.6 | HiDEF-seq v2 |
| | | | cell line | | | | |
| GM12812 | N/A | Male | Lymphoblastoid | N/A | MagAttract HMW | 8.8 | HiDEF-seq v2 |
| | | | cell line | | | | |
| 58801 | N/A | Male | Lymphoblastoid | N/A | MagAttract HMW | 8.6 | HiDEF-seq v2 |
| | | | cell line | | | | |
| 55838 | N/A | Female | Lymphoblastoid | N/A | MagAttract HMW | 9.1 | HiDEF-seq v2 |
| | | | cell line | | | | |
| 57615 | N/A | Female | Lymphoblastoid | N/A | MagAttract HMW | 8.1 | HiDEF-seq v2 |
| | | | cell line | | | | |
| 59637 | N/A | Male | Lymphoblastoid | N/A | MagAttract HMW | 9.2 | HiDEF-seq v2 |
| | | | cell line | | | | |
| SPM-1020 | 43.6 | Male | Sperm | N/A | Custom (bead milling, | 7.8 | HiDEF-seq v2 |
| | | | | | Qiagen RLT, TCEP, | | |
| | | | | | Qiaamp) | | |
| SPM-1004 | 38.8 | Male | Sperm | N/A | Custom (bead milling, | 7.5 | HiDEF-seq v2 |
| | | | | | Qiagen RLT, TCEP, | | |
| | | | | | Qiaamp) | | |
| SPM-1002 | 21.2 | Male | Sperm | N/A | Custom (bead milling, | 8.3 | HiDEF-seq v2 |
| | | | | | Qiagen RLT, TCEP, | | |
| | | | | | Qiaamp) | | |
| SPM-1013 | 18.3 | Male | Sperm | N/A | Custom (bead milling, | 8.1 | HiDEF-seq v2 |
| | | | | | Qiagen RLT, TCEP, | | |
| | | | | | Qiaamp) | | |
| SPM-1060 | 48.7 | Male | Sperm | N/A | Custom (bead milling, | 8.4 | HiDEF-seq v2 |
| | | | | | Qiagen RLT, TCEP, | | |
| | | | | | Qiaamp) | | |
| 1105 | 15.7 | Male | Blood | N/A | MagAttract HMW | 9.1 | HiDEF-seq v2 |
| 1400 | 6.3 | Male | Blood | N/A | MagAttract HMW | 8.7 | HiDEF-seq v2 |
| 1901 | 62.4 | Male | Blood | N/A | MagAttract HMW | 9.1 | HiDEF-seq v2 |
| 1901 | 62.4 | Male | Blood | N/A | MagAttract HMW −> on ice | 9.1 | HiDEF-seq v2 |
| | | | | | 6 hours (buffer: 50 mM | | |
| | | | | | KAcetate, 20 mM Tris- | | |
| | | | | | Acetate, 10 mM MgAcetate, | | |
| | | | | | 100 μg/ml albumin, pH 7.9) | | |
| 1901 | 62.4 | Male | Blood | N/A | MagAttract HMW −> 56 C. 3 | 9.1 | HiDEF-seq v2 |
| | | | | | hours/ice 3 hours (buffer: | | |
| | | | | | 50 mM KAcetate, 20 mM | | |
| | | | | | Tris-Acetate, 10 mM | | |
| | | | | | MgAcetate, 100 μg/ml | | |
| | | | | | albumin, pH 7.9) | | |
| 1901 | 62.4 | Male | Blood | N/A | MagAttract HMW −> 56 C. 6 | 9.1 | HiDEF-seq v2 |
| | | | | | hours (buffer: 50 mM | | |
| | | | | | KAcetate, 20 mM Tris- | | |
| | | | | | Acetate, 10 mM MgAcetate, | | |
| | | | | | 100 μg/ml albumin, pH 7.9) | | |
| 1901 | 62.4 | Male | Blood | N/A | MagAttract HMW −> 72 C. 3 | 9.1 | HiDEF-seq v2 |
| | | | | | hours/ice 3 hours (buffer: | | |
| | | | | | 50 mM KAcetate, 20 mM | | |
| | | | | | Tris-Acetate, 10 mM | | |
| | | | | | MgAcetate, 100 μg/ml | | |
| | | | | | albumin, pH 7.9) | | |
| 1901 | 62.4 | Male | Blood | N/A | MagAttract HMW −> 72 C. 6 | 9.1 | HiDEF-seq v2 |
| | | | | | hours (buffer: 50 mM | | |
| | | | | | KAcetate, 20 mM Tris- | | |
| | | | | | Acetate, 10 mM MgAcetate, | | |
| | | | | | 100 μg/ml albumin, pH 7.9) | | |
| 1901 | 62.4 | Male | Blood | N/A | MagAttract HMW−> on ice 6 | 9.1 | HiDEF-seq v2 |
| | | | | | hours (buffer: N/A; only | | |
| | | | | | water) | | |
| 1901 | 62.4 | Male | Blood | N/A | MagAttract HMW −> 72 C. 6 | 9.1 | HiDEF-seq v2 |
| | | | | | hours (buffer: 10 mM Tris- | | |
| | | | | | HCl, 10 mM MgAcetate, pH | | |
| | | | | | 8.0) | | |
| 1901 | 62.4 | Male | Blood | N/A | MagAttract HMW −> 72 C. 6 | 9.1 | HiDEF-seq v2 |
| | | | | | hours (buffer: 10 mM Tris- | | |
| | | | | | HCl, 10 mM MgCl2, pH 8.0) | | |
| 1901 | 62.4 | Male | Blood | N/A | MagAttract HMW −> 72 C. 6 | 9.1 | HiDEF-seq v2 |
| | | | | | hours (buffer: 10 mM Tris- | | |
| | | | | | HCl, 50 mM KCl, pH 8.0) | | |
| 1901 | 62.4 | Male | Blood | N/A | MagAttract HMW −> 72 C. 6 | 9.1 | HiDEF-seq v2 |
| | | | | | hours (buffer: 20 mM Tris- | | |
| | | | | | Acetate, 50 mM KAcetate, | | |
| | | | | | 10 mM MgAcetate, 100 | | |
| | | | | | μg/ml albumin, pH 7.9) | | |
| 1901 | 62.4 | Male | Blood | N/A | MagAttract HMW −> 72 C. 6 | 9.1 | HiDEF-seq v2 |
| | | | | | hours (buffer: 10 mM Tris- | | |
| | | | | | HCl, pH 8.0) | | |
| 1901 | 62.4 | Male | Blood | N/A | MagAttract HMW −> 72 C. 6 | 9.1 | HiDEF-seq v2 |
| | | | | | hours (buffer: N/A; only | | |
| | | | | | water) | | |
| 5203 | 4.1 | Female | Blood | N/A | MagAttract HMW | 9.0 | HiDEF-seq v2 |
| 6501 | 43.0 | Male | Blood | N/A | MagAttract HMW | 8.0 | HiDEF-seq v2 |
| 1409 | 18.1 | Male | Kidney | 6 | Nucleobond HMW, | 6.6 | HiDEF-seq v2 |
| | | | | | Magattract HMW, Qiaamp | | |
| 1324 | 13 | Male | Blood | N/A | MagAttract HMW | 9.0 | HiDEF-seq v2 |
| 1325 | 8 | Male | Blood | N/A | MagAttract HMW | 9.1 | HiDEF-seq v2 |
| 63143 | 15 | Female | Blood | N/A | MagAttract HMW | 9.1 | HiDEF-seq v2 |
| GM02036 | 11 yo at | Female | Primary fibroblasts | N/A | MagAttract HMW | 9.4 | HiDEF-seq v2 |
| | sampling | | | | | | |
| | (Passage 14) | | | | | | |
| GM03348 | 10 yo at | Male | Primary fibroblasts | N/A | MagAttract HMW | 9.4 | HiDEF-seq v2 |
| | sampling | | | | | | |
| | (Passage 7) | | | | | | |
| GM16381 | 14 yo at | Male | Primary fibroblasts | N/A | MagAttract HMW | 9.4 | HiDEF-seq v2 |
| | sampling | | | | | | |
| | (Passage 3) | | | | | | |
| 1104 | 35.3 | Male | Liver | 12 | Magattract HMW | 6.7 | HiDEF-seq v2 |
| 5697 | 67.2 | Male | Liver | 15 | Magattract HMW | 6.1 | HiDEF-seq v2 |
| 5840 | 75.4 | Male | Liver | 17 | Magattract HMW | 7.1 | HiDEF-seq v2 |
| 5344 | 25 | Female | Neurons (frontal | 4 | Qiaamp | 8.3 | HiDEF-seq v2 |
| | | | cortex) | | | | |
| 6371 | 90 | Female | Neurons (frontal | 6 | Magattract HMW | 7.9 | HiDEF-seq v2 |
| | | | cortex) | | | | |
| 63143 | N/A | Female | Lymphoblastoid | N/A | MagAttract HMW | 9.0 | HiDEF-seq v2 |
| | | | cell line | | | | |
| GM01629 | 10 yo at | Female | Primary fibroblasts | N/A | MagAttract HMW | 9.5 | HiDEF-seq v2 |
| | sampling | | | | | | |
| | (Passage 9) | | | | | | |
| GM28257 | 1 yo at | Male | Primary fibroblasts | N/A | MagAttract HMW | 9.5 | HiDEF-seq v2 |
| | sampling | | | | | | |
| | (Passage 4) | | | | | | |
| CC-346-253 | 43 | Male | Blood | N/A | MagAttract HMW | 6.7 | HiDEF-seq v2 |
| CC-388-290 | 51 | Female | Blood | N/A | MagAttract HMW | 8.9 | HiDEF-seq v2 |
| CC-713-555 | 29 | Male | Blood | N/A | MagAttract HMW | 9.1 | HiDEF-seq v2 |
| 1901 | 62.4 | Male | Blood | N/A | Puregene | 9.1 | HiDEF-seq v2 |
| 1901 | 62.4 | Male | Blood | N/A | Puregene −> 72 C. 6 hours | 9.1 | HiDEF-seq v2 |
| | | | | | (buffer: 10 mM Tris-HCl, 10 | | |
| | | | | | mM MgCl2, pH 8.0) | | |
| 1901 | 62.4 | Male | Blood | N/A | MagAttract HMW −> | 9.1 | HiDEF-seq v2 |
| | | | | | Custom (bead milling, | | |
| | | | | | Qiagen RLT, TCEP, | | |
| | | | | | Qiaamp) | | |
| D1 | 40.4 | Male | Sperm | N/A | Custom (bead milling, | 7.8 | HiDEF-seq v2 |
| | | | | | Qiagen RLT, TCEP, | | |
| | | | | | Qiaamp) | | |
| D1 | 40.4 | Male | Sperm | N/A | Custom (bead milling, | 7.6 | HiDEF-seq v2 |
| | | | | | Qiagen RLT, TCEP, | | |
| | | | | | Qiaamp) | | |
| D2 | 49.6 | Male | Sperm | N/A | Custom (bead milling, | 6.5 | HiDEF-seq v2 |
| | | | | | Qiagen RLT, TCEP, | | |
| | | | | | Qiaamp) | | |
| D2 | 49.6 | Male | Sperm | N/A | Custom (bead milling, | 7.6 | HiDEF-seq v2 |
| | | | | | Qiagen RLT, TCEP, | | |
| | | | | | Qiaamp) | | |
| SPM-1020 | 43.6 | Male | Sperm | N/A | Custom - same as sperm | 8.1 | HiDEF-seq v2 (Large |
| | | | | | DNA extraction (bead | | fragment) |
| | | | | | milling, Qiagen RLT, TCEP, | | |
| | | | | | Qiaamp) | | |
| SPM-1002 | 21.2 | Male | Sperm | N/A | Custom (bead milling, | 8.3 | HiDEF-seq v2 (Large |
| | | | | | Qiagen RLT, TCEP, | | fragment) |
| | | | | | Qiaamp) | | |
| SPM-1002 | 21.2 | Male | Sperm | N/A | Custom (bead milling, | 8.3 | Nanoseq |
| | | | | | Qiagen RLT, TCEP, | | |
| | | | | | Qiaamp) | | |
| SPM-1004 | 38.8 | Male | Sperm | N/A | Custom (bead milling, | 7.7 | Nanoseq |
| | | | | | Qiagen RLT, TCEP, | | |
| | | | | | Qiaamp) | | |
| SPM-1020 | 43.6 | Male | Sperm | N/A | Custom (bead milling, | 7.8 | Nanoseq |
| | | | | | Qiagen RLT, TCEP, | | |
| | | | | | Qiaamp) | | |
| 1443 | 0.9 | Female | Kidney | 10 | Nucleobond HMW, | 7.3 | Nanoseq |
| | | | | | Magattract HMW, Qiaamp | | |
| SPM-1013 | 18.3 | Male | Sperm | N/A | Custom (bead milling, | 8.1 | Nanoseq |
| | | | | | Qiagen RLT, TCEP, | | |
| | | | | | Qiaamp) | | |
| SPM-1060 | 48.7 | Male | Sperm | N/A | Custom (bead milling, | 8.4 | Nanoseq |
| | | | | | Qiagen RLT, TCEP, | | |
| | | | | | Qiaamp) | | |
| 1105 | 15.7 | Male | Blood | N/A | MagAttract HMW | 9.1 | Nanoseq |
| 6501 | 43.0 | Male | Blood | N/A | MagAttract HMW | 8.0 | Nanoseq |
| 63143 | 15 | Female | Blood | N/A | MagAttract HMW | 9.1 | Nanoseq |

Columns I-T

| HiDEF-seq: Chromosomes 1-22 and X | | | | | | | | | | | |
|---|---|---|---|---|---|---|---|---|---|---|---|
| | | | | | dsDNA | Nanoseq: Chromosomes 1-22 and X | | | | | |
| Number of | | ssDNA call | Number of | | mutation | | | | | | dsDNA |
| interro- | | burden | interro- | | burden | Number of | | | Number of | | mutation |
| gated | Number | (senstivity | gated | | (senstivity | interro- | Number | | interro- | | burden |
| ssDNA | of | and trinu- | dsDNA | Number of | and trinu- | gated | of | ssDNA | gated | Number of | (trinu- |
| consensus | ssDNA | cleotide | consensus | dsDNA | cleotide | ssDNA | ssDNA | call | dsDNA | dsDNA | cleotide |
| bases | calls | corrected) | base pairs | mutations | corrected) | bases | calls | burden | base pairs | mutations | corrected) |
| 4.91E+08 | 46 | 9.60E−08 | 2.90E+08 | 9 | 3.56E−08 | — | — | — | — | — | — |
| 4.54E+08 | 67 | 1.51E−07 | 2.67E+08 | 55 | 2.20E−07 | — | — | — | — | — | — |
| 4.52E+08 | 172 | 3.85E−07 | 2.66E+08 | 157 | 6.10E−07 | — | — | — | — | — | — |
| 4.54E+08 | 63 | 1.42E−07 | 2.67E+08 | 132 | 5.25E−07 | — | — | — | — | — | — |
| 1.59E+09 | 73 | 4.82E−08 | 9.45E+08 | 20 | 2.25E−08 | — | — | — | — | — | — |
| 1.71E+09 | 240 | 1.42E−07 | 1.02E+09 | 194 | 2.02E−07 | — | — | — | — | — | — |
| 1.51E+09 | 804 | 5.35E−07 | 9.00E+08 | 601 | 6.87E−07 | — | — | — | — | — | — |
| 1.71E+09 | 279 | 1.66E−07 | 1.02E+09 | 467 | 4.84E−07 | — | — | — | — | — | — |
| 1.08E+09 | 16 | 1.60E−08 | 6.94E+08 | 18 | 2.88E−08 | — | — | — | — | — | — |
| 1.59E+09 | 75 | 5.02E−08 | 1.02E+09 | 181 | 1.86E−07 | — | — | — | — | — | — |
| 5.45E+08 | 31 | 6.03E−08 | 3.47E+08 | 217 | 6.48E−07 | — | — | — | — | — | — |
| 1.53E+09 | 51 | 3.46E−08 | 9.76E+08 | 479 | 5.20E−07 | — | — | — | — | — | — |
| 1.12E+09 | 114 | 1.10E−07 | 7.18E+08 | 23 | 3.33E−08 | — | — | — | — | — | — |
| 9.24E+08 | 148 | 1.72E−07 | 5.88E+08 | 223 | 4.03E−07 | — | — | — | — | — | — |
| 1.03E+09 | 357 | 3.56E−07 | 6.58E+08 | 443 | 7.26E−07 | — | — | — | — | — | — |
| 9.53E+08 | 240 | 2.65E−07 | 6.08E+08 | 588 | 1.06E−06 | — | — | — | — | — | — |
| 1.08E+09 | 104 | 1.00E−07 | 6.16E+08 | 13 | 2.29E−08 | — | — | — | — | — | — |
| 1.13E+09 | 267 | 2.44E−07 | 6.42E+08 | 217 | 3.51E−07 | — | — | — | — | — | — |
| 1.37E+09 | 857 | 6.32E−07 | 7.83E+08 | 519 | 6.91E−07 | — | — | — | — | — | — |
| 1.15E+09 | 422 | 3.77E−07 | 6.53E+08 | 614 | 9.91E−07 | — | — | — | — | — | — |
| 8.77E+08 | 566 | 6.54E−07 | 4.96E+08 | 346 | 7.22E−07 | — | — | — | — | — | — |
| 1.05E+09 | 1449 | 1.39E−06 | 5.96E+08 | 440 | 7.58E−07 | — | — | — | — | — | — |
| 1.16E+09 | 874 | 7.57E−07 | 6.58E+08 | 517 | 8.03E−07 | — | — | — | — | — | — |
| 1.26E+09 | 1158 | 9.22E−07 | 7.17E+08 | 535 | 7.74E−07 | — | — | — | — | — | — |
| 6.40E+08 | 207 | 3.34E−07 | 3.51E+08 | 299 | 8.79E−07 | — | — | — | — | — | — |
| 5.81E+08 | 156 | 2.73E−07 | 3.19E+08 | 265 | 8.68E−07 | — | — | — | — | — | — |
| 5.55E+08 | 122 | 2.27E−07 | 3.04E+08 | 241 | 8.17E−07 | — | — | — | — | — | — |
| 5.60E+08 | 122 | 2.21E−07 | 3.07E+08 | 254 | 8.58E−07 | — | — | — | — | — | — |
| 6.33E+08 | 572 | 9.16E−07 | 3.46E+08 | 280 | 8.20E−07 | — | — | — | — | — | — |
| 6.28E+08 | 107 | 1.74E−07 | 3.38E+08 | 240 | 7.21E−07 | — | — | — | — | — | — |
| 9.35E+08 | 177 | 1.92E−07 | 5.04E+08 | 405 | 8.27E−07 | — | — | — | — | — | — |
| 1.02E+09 | 223 | 2.22E−07 | 5.50E+08 | 442 | 8.27E−07 | — | — | — | — | — | — |
| 2.85E+09 | 238 | 8.72E−08 | 1.63E+09 | 1263 | 8.03E−07 | — | — | — | — | — | — |
| 8.32E+08 | 42 | 5.23E−08 | 4.71E+08 | 330 | 7.27E−07 | — | — | — | — | — | — |
| 1.79E+09 | 141 | 8.12E−08 | 1.02E+09 | 780 | 7.85E−07 | — | — | — | — | — | — |
| 2.19E+09 | 243 | 1.15E−07 | 1.53E+09 | 276 | 2.14E−07 | — | — | — | — | — | — |
| 2.05E+09 | 178 | 8.92E−08 | 1.43E+09 | 417 | 3.51E−07 | — | — | — | — | — | — |
| 2.51E+09 | 342 | 1.42E−07 | 1.52E+09 | 140 | 9.87E−08 | — | — | — | — | — | — |
| 2.07E+09 | 219 | 1.10E−07 | 1.22E+09 | 332 | 2.92E−07 | — | — | — | — | — | — |
| 2.65E+09 | 471 | 1.92E−07 | 1.64E+09 | 35 | 2.36E−08 | — | — | — | — | — | — |
| 2.56E+09 | 490 | 2.09E−07 | 1.60E+09 | 38 | 2.64E−08 | — | — | — | — | — | — |
| 1.69E+09 | 152 | 9.45E−08 | 1.05E+09 | 185 | 2.04E−07 | — | — | — | — | — | — |
| 1.47E+09 | 35 | 2.68E−08 | 9.11E+08 | 154 | 1.98E−07 | — | — | — | — | — | — |
| 1.19E+09 | 42 | 3.73E−08 | 8.41E+08 | 507 | 6.34E−07 | — | — | — | — | — | — |
| 1.27E+09 | 83 | 6.66E−08 | 8.92E+08 | 894 | 1.02E−06 | — | — | — | — | — | — |
| 3.22E+09 | 90 | 2.92E−08 | 2.13E+09 | 281 | 1.36E−07 | — | — | — | — | — | — |
| 9.19E+08 | 46 | 5.31E−08 | 6.26E+08 | 510 | 8.81E−07 | — | — | — | — | — | — |
| 1.05E+09 | 45 | 4.61E−08 | 7.18E+08 | 641 | 9.53E−07 | — | — | — | — | — | — |
| 2.90E+09 | 213 | 7.55E−08 | 1.88E+09 | 6317 | 3.37E−06 | — | — | — | — | — | — |
| 2.97E+09 | 169 | 5.80E−08 | 1.91E+09 | 5075 | 2.70E−06 | — | — | — | — | — | — |
| 2.46E+09 | 331 | 1.56E−07 | 1.57E+09 | 36 | 2.57E−08 | — | — | — | — | — | — |
| 5.81E+09 | 836 | 1.62E−07 | 3.54E+09 | 92 | 2.92E−08 | — | — | — | — | — | — |
| 2.18E+09 | 287 | 1.45E−07 | 1.26E+09 | 17 | 1.49E−08 | — | — | — | — | — | — |
| 2.13E+09 | 229 | 1.21E−07 | 1.24E+09 | 18 | 1.54E−08 | — | — | — | — | — | — |
| 2.37E+09 | 261 | 1.25E−07 | 1.38E+09 | 36 | 2.86E−08 | — | — | — | — | — | — |
| 1.67E+09 | 51 | 3.29E−08 | 9.68E+08 | 116 | 1.32E−07 | — | — | — | — | — | — |
| 1.49E+09 | 58 | 4.20E−08 | 8.62E+08 | 505 | 6.03E−07 | — | — | — | — | — | — |
| 1.52E+09 | 59 | 4.17E−08 | 8.83E+08 | 239 | 2.97E−07 | — | — | — | — | — | — |
| 9.75E+08 | 17 | 1.95E−08 | 5.94E+08 | 171 | 3.17E−07 | — | — | — | — | — | — |
| 9.77E+08 | 43 | 5.11E−08 | 5.95E+08 | 186 | 3.50E−07 | — | — | — | — | — | — |
| 9.07E+08 | 49 | 6.40E−08 | 5.54E+08 | 172 | 3.38E−07 | — | — | — | — | — | — |
| 9.98E+08 | 233 | 2.66E−07 | 6.08E+08 | 194 | 3.53E−07 | — | — | — | — | — | — |
| 8.91E+08 | 436 | 5.58E−07 | 5.38E+08 | 160 | 3.32E−07 | — | — | — | — | — | — |
| 7.10E+08 | 13 | 1.85E−08 | 4.01E+08 | 115 | 3.03E−07 | — | — | — | — | — | — |
| 6.89E+08 | 380 | 5.89E−07 | 3.85E+08 | 107 | 2.90E−07 | — | — | — | — | — | — |
| 6.65E+08 | 376 | 6.08E−07 | 3.74E+08 | 109 | 3.03E−07 | — | — | — | — | — | — |
| 6.32E+08 | 487 | 8.13E−07 | 3.53E+08 | 99 | 2.96E−07 | — | — | — | — | — | — |
| 7.33E+08 | 368 | 5.33E−07 | 4.11E+08 | 126 | 3.19E−07 | — | — | — | — | — | — |
| 2.05E+08 | 10852 | 4.97E−05 | 1.13E+08 | 24 | 1.97E−07 | — | — | — | — | — | — |
| 7.67E+07 | 2751 | 3.31E−05 | 4.27E+07 | 16 | 3.41E−07 | — | — | — | — | — | — |
| 6.71E+08 | 14 | 2.16E−08 | 5.29E+08 | 23 | 5.12E−08 | — | — | — | — | — | — |
| 6.00E+08 | 19 | 3.61E−08 | 4.72E+08 | 119 | 2.86E−07 | — | — | — | — | — | — |
| 6.51E+08 | 71 | 1.13E−07 | 5.08E+08 | 59 | 1.33E−07 | — | — | — | — | — | — |
| 1.49E+09 | 57 | 4.01E−08 | 9.35E+08 | 475 | 5.23E−07 | — | — | — | — | — | — |
| 1.53E+09 | 60 | 4.14E−08 | 9.75E+08 | 282 | 2.97E−07 | — | — | — | — | — | — |
| 1.43E+09 | 72 | 5.20E−08 | 8.95E+08 | 1153 | 1.31E−06 | — | — | — | — | — | — |
| 1.39E+09 | 24 | 1.78E−08 | 8.20E+08 | 599 | 7.76E−07 | — | — | — | — | — | — |
| 1.50E+09 | 31 | 2.20E−08 | 8.83E+08 | 176 | 2.04E−07 | — | — | — | — | — | — |
| 1.49E+09 | 20 | 1.41E−08 | 8.77E+08 | 1115 | 1.31E−06 | — | — | — | — | — | — |
| 9.61E+08 | 438 | 4.71E−07 | 5.47E+08 | 212 | 4.03E−07 | — | — | — | — | — | — |
| 1.03E+09 | 588 | 5.80E−07 | 5.85E+08 | 451 | 7.98E−07 | — | — | — | — | — | — |
| 7.21E+08 | 422 | 6.16E−07 | 4.09E+08 | 423 | 1.06E−06 | — | — | — | — | — | — |
| 3.42E+09 | 135 | 4.24E−08 | 2.05E+09 | 148 | 7.58E−08 | — | — | — | — | — | — |
| 1.73E+09 | 81 | 4.97E−08 | 1.05E+09 | 286 | 2.96E−07 | — | — | — | — | — | — |
| 1.65E+09 | 186 | 1.15E−07 | 9.70E+08 | 8215 | 8.54E−06 | — | — | — | — | — | — |
| 1.72E+09 | 36 | 2.24E−08 | 1.01E+09 | 312 | 3.15E−07 | — | — | — | — | — | — |
| 2.08E+09 | 57 | 2.86E−08 | 1.24E+09 | 122 | 1.03E−07 | — | — | — | — | — | — |
| 1.71E+09 | 46 | 2.98E−08 | 1.08E+09 | 240 | 2.41E−07 | — | — | — | — | — | — |
| 1.89E+09 | 56 | 3.19E−08 | 1.19E+09 | 270 | 2.49E−07 | — | — | — | — | — | — |
| 1.68E+09 | 41 | 2.65E−08 | 1.05E+09 | 147 | 1.53E−07 | — | — | — | — | — | — |
| 1.44E+09 | 29 | 2.25E−08 | 9.54E+08 | 255 | 3.17E−07 | — | — | — | — | — | — |
| 7.60E+08 | 416 | 6.02E−07 | 4.47E+08 | 120 | 2.92E−07 | — | — | — | — | — | — |
| 7.56E+08 | 18 | 2.62E−08 | 4.40E+08 | 126 | 3.08E−07 | — | — | — | — | — | — |
| 7.22E+08 | 95 | 1.42E−07 | 3.98E+08 | 3 | 7.36E−09 | — | — | — | — | — | — |
| 1.03E+09 | 116 | 1.19E−07 | 5.70E+08 | 14 | 2.69E−08 | — | — | — | — | — | — |
| 1.04E+09 | 104 | 1.04E−07 | 5.78E+08 | 28 | 5.25E−08 | — | — | — | — | — | — |
| 1.18E+09 | 118 | 1.04E−07 | 6.53E+08 | 18 | 2.90E−08 | — | — | — | — | — | — |
| 1.62E+09 | 223 | 1.80E−07 | 1.83E+09 | 55 | 3.51E−08 | — | — | — | — | — | — |
| 1.81E+09 | 246 | 1.75E−07 | 2.09E+09 | 25 | 1.65E−08 | — | — | — | — | — | — |
| — | — | — | — | — | — | 1.49E+10 | 16333 | 1.09E−06 | 7.47E+09 | 112 | 1.58E−08 |
| — | — | — | — | — | — | 3.63E+09 | 3279 | 9.04E−07 | 1.81E+09 | 45 | 2.62E−08 |
| — | — | — | — | — | — | 7.29E+09 | 8101 | 1.11E−06 | 3.65E+09 | 99 | 2.97E−08 |
| — | — | — | — | — | — | 7.35E+09 | 16985 | 2.31E−06 | 3.67E+09 | 87 | 2.52E−08 |
| — | — | — | — | — | — | 1.21E+10 | 15728 | 1.30E−06 | 6.06E+09 | 97 | 1.67E−08 |
| — | — | — | — | — | — | 1.14E+10 | 9185 | 8.04E−07 | 5.71E+09 | 186 | 3.44E−08 |
| — | — | — | — | — | — | 1.43E+10 | 14548 | 1.02E−06 | 7.13E+09 | 865 | 1.31E−07 |
| — | — | — | — | — | — | 1.54E+10 | 9738 | 6.32E−07 | 7.70E+09 | 1932 | 2.71E−07 |
| — | — | — | — | — | — | 1.49E+10 | 8846 | 5.95E−07 | 7.43E+09 | 10758 | 1.44E−06 |

What is claimed is:

1. A method for determining the sequence of double stranded DNA from a plurality of nucleated cells without amplification of the DNA prior to the sequencing, the method comprising:
a) providing a biological sample comprising the plurality of nucleated cells;
b) extracting DNA from the plurality of nucleated cells;
c) fragmenting DNA extracted from the plurality of nucleated cells with either a random fragmentation method followed by exonuclease digestion to produce a plurality of blunt-ended DNA fragments, or using a restriction endonuclease to produce a plurality of blunt-ended DNA fragments;
d) exposing the plurality of DNA molecules to a ligase to repair nicks in the plurality of DNA molecules;
e) incubating the DNA fragments with a 3′-5′ exonuclease deficient polymerase and a mixture of dideoxyCTP, dideoxyGTP, and dideoxyTTP to block residual nicks, and optionally including deoxyATP to perform A-tailing;
f) circularizing the plurality of DNA fragments with hairpin adapters ligated to both ends of the DNA fragments to obtain a plurality of circularized DNA molecules;
g) sequencing each of the circularized DNA molecules individually using multiple sequencing passes for each of the circularized DNA molecules; and
h) determining the DNA sequence of each of the plurality of circularized DNA molecules, based on the sequences of each individual DNA molecule's sequencing passes.

2. The method of claim 1, wherein the determined DNA sequences are determined separately for each of the two strands of each of the plurality of double stranded DNA molecules.

3. The method of claim 2, wherein the sequence of the double strand DNA molecules comprise a double strand mutation comprising at least one nucleotide change that is present in one of the strands and a complementary nucleotide change in the complementary strand.

4. The method of claim 2, wherein the sequence of the double strand DNA molecules comprise a single strand nucleotide change comprising at least one nucleotide change that is present on only one of the two strands.

5. The method of claim 2, wherein the determined DNA sequences comprise no more than one double strand mutation for each 1 million nucleotides of determined DNA sequence base pairs, relative to a reference sequence.

6. The method of claim 2, wherein the determined DNA sequences comprise no more than one single strand nucleotide change for each 1 million nucleotides of determined DNA sequence bases.

7. The method of claim 1, wherein the A-tailing is performed.

8. The method of claim 1, wherein the sample comprises a post-mortem sample or other sample wherein DNA comprising nicks or fragments or a combination thereof is suspected to be present, and wherein the A-tailing is not performed.

9. A kit for use in the method of claim 1, wherein the kit comprises a restriction endonuclease that can produce a plurality of blunt-ended DNA fragments from a genomic DNA sample, an exonuclease that can produce a plurality of blunt-ended DNA fragments from a randomly fragmented genomic DNA sample, a 3′-5′ exonuclease deficient polymerase, and a DNA ligase.

10. The kit of claim 9, further comprising dideoxyCTP, dideoxyGTP, and dideoxyTTP in a solution or in a dry form.

11. The kit of claim 10, further comprising deoxyATP in a solution or configured in a dry form.

12. The kit of claim 11, further comprising one or more DNA oligonucleotide adapters.

13. The kit of claim 12, further comprising sealed containers that contain the restriction enzyme, the 3′-5′ exonuclease deficient polymerase, and the DNA ligase.

14. The kit of claim 13, further comprising one or more sample collection containers.

15. The kit of claim 14, further comprising printed material comprising instructions for use of the kit.
